(19) United States
(12) Patent Application Publication
(10) Pub. No.: US 2023/0407357 A1
(43) Pub. Date: Dec. 21, 2023

(54) BIOMANUFACTURING OF OLIGOSACCHARIDES AND DERIVATIVES FROM SIMPLE SUGAR

(71) Applicant: ZYMtronix Catalytic Systems, Inc., Ithaca, NY (US)

(72) Inventors: Alexander Chris Hoepker, Ithaca, NY (US); Stephane Cedric Corgie, Ithaca, NY (US); Keith Savino, Ithaca, NY (US)

(73) Assignee: ZYMtronix Catalytic Systems, Inc., Ithaca, NY (US)

(21) Appl. No.: 18/199,842

(22) Filed: May 19, 2023

Related U.S. Application Data
(60) Provisional application No. 63/430,271, filed on Dec. 5, 2022.
(60) Provisional application No. 63/344,507, filed on May 20, 2022.
(60) Provisional application No. 63/448,175, filed on Feb. 24, 2023.

(51) Int. Cl. C12P 19/28

(52) U.S. Cl. CPC C12P 19/28

(57) ABSTRACT

The invention provides methods for glycosylation and preparation of compounds. The compounds include galactosylated, sialylated, fucosylated, and N-acetylglucosaminylated compounds from simple animal-derived, plant-derived, or microbe-derived oligosaccharides and sugars. In certain embodiments, the invention provides trinucleotide-free enzymatic production of oligosaccharides starting from simple sugars that include plant-based sugars. The invention also provides the enzymatic production of fucosylated oligosaccharides and fucosylated antibody-glycan conjugates from common sugars. The production may be a cell-free, one-pot synthesis using enzymes, and in some embodiments, immobilized enzymes. The synthesis is a highly customizable and highly efficient cell-free manufacturing process. In some embodiments, lactose derivatives and human milk oligosaccharides (HMOs) are produced.

### CROSS REFERENCE TO RELATED APPLICATIONS

This application claims priority under 35 U.S.C. § 119(e) to U.S. Provisional Application No. 63/430,271, filed Dec. 5, 2022, to U.S. Provisional Application No. 63/344,507, filed May 20, 2022, and to U.S. Provisional Application No. 63/448,175, filed Feb. 24, 2023, and each are incorporated herein by reference in their entirety.

### FIELD OF THE INVENTION

The invention provides methods for glycosylation and preparation of compounds. The compounds include galactosylated, sialylated, fucosylated, and N-acetyl glucosaminylated compounds from simple animal-derived, plant-derived, or microbe-derived oligosaccharides and sugars. In certain embodiments, the invention provides trinucleotide-free enzymatic production of oligosaccharides starting from simple sugars that include plant-based sugars. The production may be a cell-free, one-pot synthesis using enzymes, and in some embodiments, immobilized enzymes. The synthesis is a highly customizable and highly efficient cell-free manufacturing process. In some embodiments, lactose derivatives and human milk oligosaccharides (HMOs) are produced.

The invention also provides the enzymatic production of fucosylated oligosaccharides and fucosylated antibody-glycan conjugates from common sugars. The production is a cell-free, one-pot synthesis using immobilized enzymes. The oligosaccharide synthesis is a highly customizable and highly efficient cell-free manufacturing process. In some embodiments, fucosylated human milk oligosaccharides (HMOs) are produced.

### BACKGROUND OF THE INVENTION

Human milk oligosaccharides (HMOs) are particularly commercially relevant glycan models. HMOs are structurally diverse, soluble and unconjugated glycans composed of between 3-22 sugars limited to a set of only 5 monosaccharides: D-glucose, D-galactose, N-acetylglucosamine, L-fucose, and N-acetylneuraminic acid (i.e., sialic acid), and are the third largest component of breast milk. Simple HMOs (3-4 DP) (DP is degree of polymerization) serve as metabolic substrates for specialized beneficial bacteria, thereby shaping the intestinal microbiome. More complex and branched HMOs (>4 DP), however, serve as soluble decoys for viral, bacterial, or protozoan parasite adhesins, thereby preventing attachment to the infant mucosal surface. For example, the fucosylated fraction of human milk was shown to inhibit Campylobacter jejuni colonization ex vivo (human intestinal mucosa) and in rodents (in vivo)— C. jejuni is a leading cause of bacterial diarrhea with and incidence of roughly 1 in 4,000 among infants <1 year of age. In addition to their protective effects against Campylobacter, evidence suggests fucosylated HMOs can deploy similar decoy mechanisms to protect infants from infection by other diarrhea-causing pathogens (e.g., cholera and norovirus) that initiate infection by binding cell-surface receptors. These oligosaccharides may also provide protection from Shiga toxin-induced diarrhea by competing with the toxin for binding to its extracellular target. Moreover, HMOs can also modulate epithelial and immune cell responses and reduce excessive mucosal leukocyte infiltration and activation. These properties have been associated with a lower risk for developing necrotizing enterocolitis.

While milk oligosaccharides form a diverse family of carbohydrates highly conserved in mammals, the profiles are not uniform amongst all species. Typically consisting of a lactose (gal+glu) core, elongated by alternate N-acetylglucosamine and galactose units, and decorated by sialylation and/or fucosylation. HMOs exhibit greater diversity than oligosaccharides found in bovine milk, the basis for most commercial baby formulas. At 0.05 g/L, the compounds are a minor component of cow's milk compared to the concentrations found in human milk which, at 5-23 g/L, puts HMOs on a par with protein content (8 g/L). Roughly 200 HMO structures have been identified, whereas less than half that number have been found in cows.

The global Human Milk Oligosaccharides (HMO) market size is anticipated to expand at a compound annual growth rate (CAGR) of 21.9% over the next 8 years, reaching USD 71.2 billion by 2025.38 The recent manufacturing at industrial scale of fucosylated lactose (2′FL) via fermentation with recombinant microorganisms has unlocked the market for HMOs. Regarding HMO product types, the fucosyllactose (2′FL) segment is currently largest with a CAGR of 14.4%, while the sialyllactose segment has a CAGR of 15.1%.38 The functional food and beverages sector is also expected to experience considerable growth during this period as HMOs have numerous potential applications besides infant formula.

There is a need for improved HMO synthesis methods driven by usage of HMO in infant formulas. Key drivers of growth include growing consumer awareness and concern regarding infant health, rise in demand for infant nutrition food products, projected population growth, as well as increasing awareness of health benefits associated with the consumption of HMOs, such as promotion of beneficial microbiota in the digestive tract and enhanced brain and immune system development. On the other hand, scarce raw materials, costly and complex chemical and enzymatic synthesis techniques and lack of technical expertise are expected to continue to present challenges.

While some simple probiotic HMOs can now be effectively produced via fermentation for infant formula (2′FL, 3DP), complex and branched HMOs are elusive and have been marginally produced at high cost via chemocatalysis. To make HMOs, sugars need to be first activated to be able to be sequentially transferred to the growing structures. For example, activated fucose (L-Fucose-1-GDP) and activated sialic acid (Sialic acid-1-CMP) cost $35,000 and $8,000/g respectively, while L-fucose and L-sialic acid cost $10 and $5.5/g, putting the cost of any complex glycans in hundreds of thousand dollars per gram−the cost of defined pentaoses (5 carbon sugars) DP5 glycans for example ranges from $300,000 to $3,800,000 per gram. Production of HMOs has been achieved in three main ways besides extraction from rare human milk:

Chemical production: The chemical synthesis of HMOs is flexible but entails a larger number of steps, poor yields and specialized equipment that results in high costs of production. Furthermore, complex HMOs require the synthesis of chemically protected monosaccharide that each require laborious deprotections adding up to typically 20-50 chemical manipulations (Lacto-N-tetraose synthesis). For these reasons, large-scale production of HMOs has not been implemented.

Microbe-based production: Fermentation is a cost-effective method of HMO production that is currently used to produce 2′-fucosyllactose (2′-FL) at a multi-ton industrial scale (Abbott Laboratories-Jennewein Biotechnologie GmbH, Glycom-Nestle, Inbiose-DuPont). Microbial production, however, is limited to simple unbranched and short-length (DP<4) HMOs. More complex HMOs are out of reach because of poor yields for complex carbohydrates via fermentation and the challenges of controlling the glycan sequences in living organisms.

Enzyme-based production: Cell-free production circumvents this limitation by using a glycosyltransferase that transfers a nucleotide activated donor sugar (e.g. GDP-L-fucose, CMP-sialic acid etc.) with high specificity to a large variety of acceptor sugars. Combining various enzymatic steps can produce large HMO libraries, an approach that has been taken by HMO commercial suppliers (Chemily Glycoscience, Carbosynth, Santa Cruz Biotechnology). With fewer steps and robust enzymes, chemocatalysis can be used to produce defined complex glucans but still suffer from high costs of production.

Biocatalysis, as a green technology, has become increasingly popular in chemical manufacturing over traditional expensive and inefficient processes. Its applications include the production of food ingredients, flavors, fragrances, commodity and fine chemicals, and active pharmaceuticals. When producing chemicals at industrial scale, however, enzymes can suffer drastic losses in activity and loading causing a significant drop in performance.

Continuous flow processing begins with two or more streams of different reactants pumped at specific flow rates into a single cell. A reaction takes place, and the stream containing the resultant compound is collected at the outlet. The solution may also be directed to subsequent flow reactor loops to generate the final product. Continuous flow processing provides better control and reproducibility of reactions. It is a modular, customizable approach. The high modularity allows one to configure the cells to meet the requirements of specific reactions.

Glycans are complex carbohydrate structures that are the predominant molecules on the cell surface and serve as the first point of contact between cells, the extracellular matrix and pathogens. Glycans are involved in a number of biological processes such as cell-to cell-interactions. There is great interest in improving the accessibility and affordability of these molecules for research, preclinical and commercial applications.

A recent study by the CDC found that roughly 1-in-5 mothers of newborns never initiate breastfeeding and the majority (>70%) do not meet the 6-month target, instead relying extensively or exclusively on commercially available infant formula. Non-compliance with breastfeeding recommendations is estimated to add an additional $2.5 billion to pediatric healthcare costs (direct) in the US with the total burden of morbidity and mortality totaling $13.8 billion. (Vera MK NG, “Implementation of Mother-friendly Workplace Policies in Hong Kong”, Hong Kong J Gynaecol Obstet. Midwifery 2015; 15(1) 11.

Human milk oligosaccharides (HMOs) are the third largest component of breast milk. They are of particular commercial relevance. For instance, they serve as metabolic substrates for specialized beneficial bacteria, thereby shaping the intestinal microbiome. More complex and branched HMOs (>4 DP (i.e., degree of polymerization or the number of monosaccharides linked together), however, appear mostly prophylactic. They serve as soluble decoys for viral, bacterial, or protozoan parasite adhesins and prevent their attachment to the infant, or adults, mucosal surfaces.

Moreover, HMOs can also modulate epithelial and immune cell responses and reduce excessive mucosal leukocyte infiltration and activation. These properties have been associated with a lower risk for developing necrotizing enterocolitis and other infections and autoimmune inflammations.

While some simple probiotic HMOs can be effectively produced via fermentation for infant formula (2′FL, 3DP), complex and branched HMOs are elusive. Microbial production is limited to simple unbranched and short-length sugars and bacteria can secrete toxins that must be filtered out. Yeast fermentation does not require the removal of toxins, and the overall process involves fewer steps, reducing production costs and resulting in a more easily scaled product. Engineering organisms is intensive and does not guarantee the ability to scale-up.

Cell free systems have been of interest in biomanufacturing, but there are challenges (M. P. Cordoso Marque et al., Adv. Biochem. Eng. Biotechnol., https://doi.org/10.1007/10_2020_160 and C. You, Adv. Biochem. Engl, DOI: 10.1007/10_2012_159 (2012)). Hokke, Cornelis H., et al. “One-pot enzymatic synthesis of the Galα1→3Galβ1→4GlcNAc sequence within situ UDP-Gal regeneration.” Glycoconjugate journal 13.4 (1996): 687-692. Zervosen, Astrid, and Lothar Elling. “A Novel Three-Enzyme Reaction Cycle for the Synthesis of N-Acetyllactosamine with in Situ Regeneration of Uridine 5′-Diphosphate Glucose and Uridine 5′-Diphosphate Galactose.” Journal of the American Chemical Society 118.8 (1996): 1836-1840. The foregoing are incorporated by reference in their entirety.

Thus, the art seeks an economical and efficient way to produce glycans. Thus, the art seeks an economical and efficient way to produce glycans, including HMOs, including, but not limited to, glycans of two sugar units or larger, and including glycans of 4 sugar units or larger. This includes HMOs including, but not limited to, glycans of two or four sugar units or larger. This could overcome major hurdles in advancing these glycans, galactosylated glycan, and HMOs for probiotic, prophylactic, and therapeutic purposes. This includes, but is not limited to, medical foods, food additives, and supplements. The economical and efficient HMO production would be an important advance for global infant nutrition and disease prevention for all ages. Importantly, the invention provides enhanced production efficiency of sugars and complex carbohydrates, while lowering costs, hence improving the accessibility and affordability of these molecules.

### SUMMARY OF THE INVENTION

The present invention provides processes for preparing saccharides starting from common sugar building blocks. This lowers the cost of saccharide production. In certain embodiments the sugars are plant derived. The energy source is hydrolysis of more complex sugars into more simple sugars. Activated sugars are the starting materials and are generated in situ. The activated sugars are monophosphates or diphosphates present in catalytic amounts. Examples include the nucleotide di- and monophosphates UDP, GDP, and CMP. Avoiding stoichiometric or excess nucleotide reactants, including triphosphates such as ATP, avoids the need for co-factor recycling, reduces the number of enzymes required, avoids reaction inhibition, and lowers the overall cost of the reaction over current reactions. Nucleotide di- and monophosphate are used in catalytic amounts. This is a significant improvement over prior art reactions where ATP must be used in stoichiometric amounts or in excess.

The present invention provides methods with improved purification and yields. There is no need for acidification that leads to improved purification. Additionally, there are no inorganic phosphate biproducts to purify from the reaction mixture. In certain embodiments, sugar biproducts are formed that are useful as building blocks for various uses. Enzymes are not reactivated thus avoiding additional reactants while improving reaction efficiency. The invention provides a cell free biomanufacturing method for making human milk oligosaccharides and other animal-based oligosaccharides from cost-effective starting materials. In some embodiments, the reactions are done as a one-pot synthesis.

Any compound with at least one hydroxy functional group may be glycosylated according to this invention. This includes, but is not limited to, saccharides, glycans, carbohydrates, and alcohols.

Thus, in some embodiments, the invention provides a method for producing a glycosylated principal product, comprising the steps of:
a. contacting a catalytic amount of a sugar-nucleotide donor and a stoichiometric amount of an acceptor in the presence of a transferase to obtain a glycosylated principal product and a catalytic amount of a nucleotide; and
b. regenerating the nucleotide into a regenerated sugar-nucleotide donor by contacting the catalytic amount of the nucleotide with a stoichiometric amount of a sugar donor precursor in the presence of a transferase to obtain the regenerated sugar-nucleotide donor and a secondary product.

In some embodiments, the method further comprises:
a. contacting a catalytic amount of the regenerated sugar-nucleotide donor and a stoichiometric amount of an acceptor in the presence of a transferase to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor in the presence of a transferase to obtain the regenerated sugar-nucleotide donor and the secondary product.

In other embodiments, the invention provides a method for producing a glycosylated principal product comprising the step of adding a sugar donor precursor, an acceptor, a transferase, and a catalytic amount of a nucleotide to obtain a glycosylated principal product and a secondary product. In certain embodiments, the sugar donor precursor and the nucleotide provide a sugar-nucleotide donor.

In certain embodiments, the sugar donor precursor and the nucleotide provide a sugar-nucleotide donor precursor and an auxiliary enzyme and the sugar-nucleotide donor precursor provide the sugar-nucleotide donor.

In some embodiments, the sugar donor precursor is a sugar comprising 2 or more sugar units. In some embodiments, the sugar donor precursor is a sugar comprising 2, 3, or 4 sugar units. In some embodiments, sugar donor precursor is a disaccharide. In some embodiments, the sugar donor precursor is a trisaccharide. In some embodiments, the sugar donor precursor comprises a galactose, sialic acid, fucose, or N-acetylglucosamine. In some embodiments, the galactose, sialic acid, fucose, or N-acetylglucosamine is in a terminal position in the sugar donor precursor.

In some embodiments, the sugar-nucleotide donor comprises a galactose, sialic acid, fucose, or N-acetylglucosamine. In some embodiments the sugar-nucleotide donor is a galactosyl-nucleotide, sialyl-nucleotide, fucosyl-nucleotide, or N-acetylglucosaminyl-nucleotide. In some embodiments, the secondary product is a monosaccharide, disaccharide, or trisaccharide.

Some embodiments of this invention further comprise the step of contacting the secondary product and a processing enzyme to convert the secondary product to a secondary product derivative. In some embodiments, the secondary product derivative, in the presence of a second processing enzyme, is converted to a modified secondary product derivative. Some embodiments of this invention further comprise the step of contacting the principal product and a processing enzyme to convert the principal product to a principal product derivative. In some embodiments, the processing enzyme is an oxidase, an isomerase, or a hydrolase.

In some embodiments, the acceptor is an organic compound containing a hydroxyl group. In some embodiments, the organic compound containing a hydroxy group is a sugar. In some embodiments, the acceptor is obtained from the processing enzyme converting the secondary product into the acceptor.

In some embodiments, the nucleotide is a uridine diphosphate, cytidine monophosphate, or guanosine diphosphate. In some embodiments, the nucleotide is in an amount of 0.001 mol percent to 10 mol percent relative to the sugar donor precursor or the acceptor. In some embodiments, the nucleotide is in an amount of 0.01 mol percent to 1 mol percent relative to the sugar donor precursor or the acceptor.

In certain embodiments, the transferase is any enzyme that transfers a sugar unit to the acceptor. In some embodiments, the transferase is a β-1,3-galactosyl transferase, sucrase synthase, β-1,4-galactosyl transferase, sialyl transferase, fucosyl transferase, or glucosaminyl (N-acetyl) transferase 2. In some embodiments, the transferase comprises a first transferase (GT1 in FIG. 6, 7, 13, or 14) and a second transferase (GT2 in FIG. 6, 7, 13, or 14). In some embodiments, the first transferase catalyzes a transfer of a sugar from the sugar-nucleotide donor to the acceptor to obtain the glycosylated principal product and the second transferase catalyzes a reaction of the nucleotide and the sugar donor precursor to obtain the sugar-nucleotide donor and the secondary product.

In certain embodiments, this invention provides a method for producing a galactosylated principal product, comprising the steps of:
a. contacting a catalytic amount of a sugar-nucleotide donor and a stoichiometric amount of an acceptor to obtain a galactosylated principal product and a catalytic amount of a nucleotide; and
b. regenerating the nucleotide into a regenerated sugar-nucleotide donor by contacting the catalytic amount of the nucleotide with a stoichiometric amount of a sugar donor precursor to obtain the regenerated sugar-nucleotide donor and a secondary product.

In some embodiments, the method further comprises:
a. contacting a catalytic amount of the regenerated sugar-nucleotide donor and a stoichiometric amount of an acceptor to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor to obtain the regenerated sugar-nucleotide donor and the secondary product.

In some embodiments, the invention provides a method for producing a galactosylated principal product comprising the step of adding a sugar donor precursor, an acceptor, a transferase, and a catalytic amount of a nucleotide to obtain a glycosylated principal product and a secondary product.

In some embodiments, the acceptor is lacto-N-triose II (LNTII), glucose, or galactooligosaccharide (GOS) and the secondary product is fructose. Some embodiments comprised the step of obtaining the glucose by contacting the fructose in the presence of a glucose isomerase to obtain the glucose. Some embodiments comprise the step of obtaining the LNTII by contacting lactose and N-acetylglucosamine in the presence of β-N-acetylhexosaminidase (Bbh1) to obtain the LNTII.

In some embodiments, the sugar donor precursor is sucrose. In some embodiments, the sugar donor precursor and the nucleotide provides a sugar-nucleotide donor precursor and wherein an auxiliary enzyme and the sugar-nucleotide donor precursor provides the sugar-nucleotide donor. In some embodiments, the sugar-nucleotide donor precursor is a glucose nucleotide. In some embodiments, the secondary product is fructose. In some embodiments, the auxiliary enzyme is a galactose epimerase. In some embodiments, the nucleotide is uridine diphosphate.

In certain embodiments, the transferase is any enzyme that transfers a galactose unit to the acceptor. In some embodiments, the transferase comprises a first transferase (GT1 in FIG. 6, 7, 13, or 14) and a second transferase (GT2 in FIG. 6, 7, 13, or 14). In particular embodiments, the first transferase catalyzes a transfer of a sugar from the sugar-nucleotide donor to the acceptor to obtain the galactosylated principal product and the second transferase catalyzes a reaction of the nucleotide and the sugar donor precursor to obtain the sugar-nucleotide donor and the secondary product. In some embodiments, the first transferase is β-1,3-galactosyl transferase from Chromobacterium violaceum (Cvb3GalT), β-1,4-Galactosyltransferase from Neisseria meningitidis (NmLgtB), and the second transferase is sucrose synthase from Arabidopsis thaliana (AtSuSy1).

In some embodiments, the galactosylated principal product is lacto-N-tetraose (LNT), Lacto-N-neotetraose (LNnT), lactose, or galactooligosaccharide (GOS). In some embodiments, the methods comprise the step of contacting the secondary product or the principal product and a processing enzyme to convert the secondary product to a secondary product derivative or the principal product to a principal product derivative. In some embodiments, the processing enzyme is an oxidase, an isomerase, or a hydrolase. In some embodiments, the processing enzyme is an oxidase and galactosylated principal product derivative is oxidized fructose or oxidized glucose.

In some embodiments, the invention provides a method for producing a sialylated principal product, comprising the steps of:
a. contacting a catalytic amount of a sugar-nucleotide donor and a stoichiometric amount of an acceptor to obtain a sialylated principal product and a catalytic amount of a nucleotide; and
b. regenerating the nucleotide into a regenerated sugar-nucleotide donor by contacting the catalytic amount of the nucleotide with a stoichiometric amount of a sugar donor precursor to obtain the regenerated sugar-nucleotide donor and a secondary product.

In some embodiments, the method of further comprises:
a. contacting a catalytic amount of the regenerated sugar-nucleotide donor and the stoichiometric amount of an acceptor to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor to obtain the regenerated sugar-nucleotide donor and the secondary product.

In some embodiments, the invention provides a method for producing a sialylated principal product comprising the step of adding a sugar donor precursor, an acceptor, a transferase, and a catalytic amount of a nucleotide to obtain a sialylated principal product and a secondary product.

In some embodiments, the sugar donor precursor is 3′-sialyllactose. In some embodiments, the sugar-nucleotide donor is a N-acetyl neuraminic acid-nucleotide. In some embodiments, the secondary product is lactose. In some embodiments, the sialylated principal product is Sialyllacto-N-tetraose a (LSTa), Sialyliacto-N-tetraose b (LSTb), Siaiyliacto-N-neoteiraose c (LSTc), Sialyilacio-N-neotetraose d (LSTd), or Disialyllacto-N-tetraose (DSLNT). In some embodiments, the secondary product or the principal product and a processing enzyme to convert the secondary product to a secondary product derivative or the principal product to a principal product derivative. In some embodiments, the processing enzyme is an oxidase, an isomerase, or a hydrolase. In some embodiments, the secondary product, in the presence of a processing enzyme, is converted to a secondary product derivative. In some embodiments, the processing enzyme is lactase. In some embodiments, the secondary product derivative is glucose and galactose. In some embodiments, the processing enzyme is an enzyme that oxidizes the lactose to lactobionic acid. In some embodiments, the processing enzyme is a lactose oxidase. In some embodiments, the glucose, in the presence of a second processing enzyme, is converted to a modified secondary product derivative. In some embodiments, the second processing enzyme is D-galactose isomerase and the modified secondary product derivative is tagatose. In some embodiments, the galactose isomerase is sourced from Geobacillus stearothermophilus (GsAI). In some embodiments, the glucose oxidase and the modified secondary product derivative is gluconolactone. In some embodiments, the processing enzyme is an oxidase, isomerase, or hydrolase and the principal product derivative is an oxidized principal product, isomerized principal product, or a hydrolyzed principal product. In some embodiments, the hydrolase is α2-3 Neuraminidase S. In some embodiments, the α2-3 Neuraminidase S and the principal product DSLNT is converted to LSTb by the neuraminidase.

In some embodiments, the principal product derivative is oxidized, isomerized or hydrolyzed LSTa, LSTb, LSTc, LSTd, or DSLNT. In some embodiments, the nucleotide is cytidine monophosphate.

In certain embodiments, the transferase is any enzyme that transfers a sialyl unit to the acceptor. In some embodiments, the transferase comprises a first transferase (GT1 in FIG. 6, 7, 13, or 14) and a second transferase (GT2 in FIG. 6, 7, 13, or 14. In particular embodiments, the first transferase catalyzes a transfer of a sugar from the sugar-nucleotide donor to the acceptor to obtain the sialylated principal product and the second transferase catalyzes a reaction of the nucleotide and the sugar donor precursor to obtain the sugar-nucleotide donor and the secondary product. In some embodiments, the first transferase is beta-galactoside alpha-2,6-sialyltransferase 1 (ST6GAL1), CMP-N-acetylneuraminate-beta-1,4-galactoside alpha-2,3-sialyltransferase 3 (ST3GAL3) or Alpha-N-acetylgalactosaminide alpha-2,6-sialyltransferase 5 (ST6GALNAC5) and second transferase is CMP-N-acetylneuraminate-beta-galactosamide-alpha-2,3-sialyltransferase 4 (ST3GAL4). In some embodiments, the first transferase ST3GAL3 and ST6GALNAC5.

In some embodiments, the invention provides a method for producing a fucosylated principal product, comprising the steps of:
a. contacting a catalytic amount of a sugar-nucleotide donor and a stoichiometric amount of an acceptor to obtain a fucosylated principal product and a catalytic amount of a nucleotide; and
b. regenerating the nucleotide into a regenerated sugar-nucleotide donor by contacting the catalytic amount of the nucleotide with a stoichiometric amount of a sugar donor precursor to obtain the regenerated sugar-nucleotide donor and a secondary product.

In some embodiments, the method further comprises:
a. contacting a catalytic amount of the regenerated sugar-nucleotide donor and the stoichiometric amount of an acceptor to obtain the glycosylated principal product and the catalytic amount of a nucleotide; and
b. regenerating the nucleotide into the regenerated sugar-nucleotide donor by contacting the catalytic amount of the nucleotide with the stoichiometric amount of a sugar donor precursor in the presence of a transferase to obtain the regenerated sugar-nucleotide donor and the secondary product.

In some embodiments, the invention provides a method for producing a fucosylated principal product comprising the step of adding a sugar donor precursor, an acceptor, a transferase, and a catalytic amount of a nucleotide to obtain a fucosylated principal product and a secondary product. In some embodiments, the sugar donor precursor is a 2′-fucosyllactose. In some embodiments, the sugar-nucleotide donor is a fucose nucleotide. In some embodiments, the secondary product is lactose.

In some embodiments, the principal product is Lacto-N-fucopentaose I (LNFPI), Lacto-N-fucopentaose II (LNFPII), Lacto-N-fucopentaose III (LNFPIII), Difucosyllactose (DFL), or 3-fucosyllactose (3-FL).

In some embodiments, the method further comprises the step of contacting the secondary product or the principal product and a processing enzyme to convert the secondary product to a secondary product derivative or the principal product to a principal product derivative. In some embodiments, the processing enzyme is an oxidase, an isomerase, or a hydrolase. In some embodiments, the secondary product, in the presence of a processing enzyme, is converted to a secondary product derivative. In some embodiments, the processing enzyme is an enzyme that oxidizes the lactose to lactobionic acid. In some embodiments, the processing enzyme is lactose oxidase. In some embodiments, the processing enzyme is lactase. In some embodiments, the secondary product derivative is glucose and galactose. In some embodiments, the secondary product derivative, in the presence of a second processing enzyme, is converted to a modified secondary product derivative. In some embodiments, the second processing enzyme is a D-galactose isomerase the modified secondary product derivative is tagatose. In some embodiments, the D-galactose isomerase is sourced from Geobacillus stearothermophilus (GsAI). In some embodiments, the second processing enzyme is glucose oxidase and the modified secondary product derivative is gluconolactone.

In some embodiments, the principal product, in the presence of a processing enzyme, is converted to a principal product derivative. In some embodiments, the processing enzyme is an oxidase, isomerase, or hydrolase and the principal product derivative is an oxidized principal product, isomerized principal product, or a hydrolyzed principal product. In some embodiments, the principal product derivative is oxidized, isomerized or hydrolyzed LNFPI, LNFPII, LNFPIII, DFL, or 3-FL. In some embodiments, the methods comprise the further step of oxidizing the fucosylated principal product in the presence of an oxidase to provide a principal product derivative. In some embodiments, the principal product derivative is oxidized LNFPI, LNFPII, LNFPIII, DFL, or 3-FL. In some embodiments, the nucleotide is guanosine diphosphate.

In certain embodiments, the transferase is any enzyme that transfers a fucose unit to the acceptor. In certain embodiments, the transferase is any enzyme that converts the sugar donor precursor into a sugar nucleotide donor and a secondary product. In some embodiments, the transferase comprises a first transferase (GT1 in FIG. 6, 7, 13, or 14) and a second transferase (GT2 in FIG. 6, 7, 13, or 14). In particular embodiments, the first transferase catalyzes a transfer of a sugar from the sugar-nucleotide donor to the acceptor to obtain the fucosylated principal product and the second transferase catalyzes a reaction of the nucleotide and the sugar donor precursor to obtain the sugar-nucleotide donor and the secondary product. In some embodiments, the first transferase is α-1,2-fucosyltransferase from Thermosynechococcus vestitus (Te2FT), al-3/4-fucosyltransferase from Helicobacter pylori (Hp34FT), fucosyl transferase 9 (FUT9), or fucosyl transferase 3 (FUT3) and the second transferase is α-1,2-fucosyltransferase from Helicobacter mustelae (HmFucT) or fucosyl transferase (FUT1).

In some embodiments, this invention provides a method for producing a N-acetylglucosaminylated principal product, comprising the steps of:
a. contacting a catalytic amount of a sugar-nucleotide donor to a stoichiometric amount of an acceptor to obtain a N-acetylglucosaminylated principal product and a catalytic amount of a nucleotide; and
b. regenerating the nucleotide into a regenerated sugar-nucleotide donor by contacting the catalytic amount of the nucleotide with a stoichiometric amount of a sugar donor precursor to obtain the regenerated sugar-nucleotide donor and a secondary product.

In some embodiments, the method further comprises:
a. contacting a catalytic amount of the regenerated sugar-nucleotide donor to the stoichiometric amount of an acceptor to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor to obtain the regenerated sugar-nucleotide donor and the secondary product.

In some embodiments, the invention provides a method for producing a N-acetylglucosaminylated principal product comprising the step of adding a sugar donor precursor, an acceptor, a transferase, and a catalytic amount of a nucleotide to obtain a N-acetylglucosaminylated principal product and a secondary product.

In some embodiments, the sugar donor precursor is lacto-N-biose. In some embodiments, the sugar-nucleotide donor is a N-acetylglucosamine nucleotide. In some embodiments, the secondary product is galactose. In some embodiments, the principal product is LNTII or β-1,6-GlcNAc-LNnT. In some embodiments, the methods further comprise the step of contacting the secondary product or the principal product and a processing enzyme to convert the secondary product to a secondary product derivative or the principal product to a principal product derivative. In some embodiments, the processing enzyme is an oxidase, an isomerase, or a hydrolase. In some embodiments, the secondary product, in the presence of a processing enzyme, is converted to a secondary product derivative. In some embodiments, the processing enzyme is a D-galactose isomerase the secondary product derivative is tagatose. In some embodiments, the D-galactose isomerase is sourced from Geobacillus stearothermophilus (GsAI). In some embodiments, the methods comprise the further step of oxidizing the N-acetylglucosaminylated principal product in the presence of an oxidase to provide a principal product derivative. In some embodiments, the principal product derivative is oxidized the principal product is LNTII or β-1,6-GlcNAc-LNnT.

In some embodiments, the nucleotide is uridine diphophate.

In certain embodiments, the transferase is any enzyme that transfers a N-acetylglucosamine unit to the acceptor. In some embodiments, the transferase is β-1,3-N-Acetyl-Hexosaminyl-transferase from Neisseria meningitidis (NmLgtA). In some embodiments, the transferase comprises a first transferase (GT1 in FIG. 6, 7, 13, or 14) and a second transferase (GT2 in FIG. 6, 7, 13, or 14. In particular embodiments, the first transferase catalyzes a transfer of a sugar from the sugar-nucleotide donor to the acceptor to obtain the N-acetylglucosaminylated principal product and the second transferase catalyzes a reaction of the nucleotide and the sugar donor precursor to obtain the sugar-nucleotide donor and the secondary product. In some embodiments, the first transferase is glucosaminyl (N-acetyl) transferase 2 (GCNT2) and second transferase is β-1,3-N-Acetyl-Hexosaminyl-transferase from Neisseria meningitidis (NmLgtA). In some embodiments, the further step of oxidizing the N-acetylglucosaminylated principal product in the presence of an oxidase to provide a principal product derivative. In some embodiments, the principal product derivative is oxidized LNTII or oxidized β-1,6-GlcNAc-LNnT. In some embodiments, the sugar is in a naturally occurring isomeric form.

In some embodiments, glucose is D-glucose. In some embodiments, is D-galactose. In some embodiments, fucose is L-fucose.
1. In some embodiments, this invention provides a compound prepared by a method according to any of the methods herein. In some embodiments, this invention provides a glycosylated compound prepared according to any of the methods of this invention. In some embodiments, this invention provides a galactosylated compound prepared according any of the methods of this invention. In some embodiments, this invention provides a sialylated compound prepared according to any of the methods of this invention. In some embodiments, this invention provides a fucosylated compound prepared according to any of the methods of this invention. In some embodiments, this invention provides a N-acetylglucosaminylated compound prepared according to any of the methods of this invention. In some embodiments, this invention provides a lactose derivative prepared according to any of the methods of this invention. In some embodiments, this invention provides lactose derivative according to any of the methods of this invention selected from lactobionic acid, lactitol, lactosucrose, galacto-oligosaccharides, lactulose, and an HMO. In some embodiments, this invention provides a lactose, DSLNT, LNnT, LSTs, LSTb, LSTc, or LSTd. In some embodiments, the compounds are obtained from non-animal based plant materials.

In some embodiments, the invention provides a machine configured for the method of any embodiment. In certain embodiments, a method is within a single reaction vessel. Also provided by this invention is a machine optimized for the processes of this invention. In certain embodiments, the machine is adapted so that the process is carried out within a single reaction vessel.

In some embodiments of the invention, one or more the enzymes are immobilized. In other embodiments, each enzyme is immobilized. In other embodiments of the invention, the process occurs in a single reaction vessel.

The invention also provides compounds prepared according to the processes of the invention. In one embodiment, LNT is prepared by a process of this invention. In another embodiment, LNnT is prepared by a process of this invention. In another embodiment, Lactose is prepared by a process of this invention. In another embodiment, a galactooligosaccharide is by a process of this invention. In another embodiment, LSTa is prepared by a process of this invention. In another embodiment, LSTb is prepared by a process of this invention. In another embodiment, LSTc is prepared by a process of this invention. In another embodiment, LSTd is prepared by a process of this invention. In another embodiment, DSLNT is prepared by a process of this invention. In another embodiment, LNFPI is prepared by a process of this invention. In another embodiment, LNFPII is prepared by a process of this invention. In another embodiment, LNFPIII is prepared by a process of this invention. In another embodiment, DFL is prepared by a process of this invention. In another embodiment, 3-FL is prepared by a process of this invention. In another embodiment, LNTII is prepared by a process of this invention. In another embodiment, a branched HMO is prepared by a process of this invention.

Any of the embodiments of this invention may be employed to obtain a compound from non-animal based plant materials.

In another embodiment, glycosylation reactions employing catalytic nucleotides are done in combination to provide a glycosylated principal product with two (2) or more sugar units that have been transferred to an acceptor via two (2) or more sugar-nucleotide donors of this invention.

### Integrated Fucosylation Pathway

In another aspect, the present invention provides an integrated pathway using immobilized enzymes to perform syntheses of fucosolyated saccharides starting from common sugars, including one-pot syntheses of fucosylated saccharides. Included is a cell free biomanufacturing method of making fucosylated human milk oligosaccharides and other fucosylated oligosaccharides from fructose, glucose or a mixture of fructose and glucose. Starting from common sugars lowers the cost of production of fucosylated HMOs vs using fucose as a starting material. FIG. 15-18 depict integrated fucosylation pathways of this invention.

The invention provides direct production of fucosylated HMOs starting from cost effective glucose or fructose instead of fucose. Regeneration of cofactors across the enzyme reactions drives efficiency and cost reduction. The production method allows the flexibility of production of all fucosylated HMOS (or oligosaccharides) by changing the last transferase enzyme and the acceptor backbone (biorefinery concept). The process does not require genetically modified organisms (non-GMO).

In some embodiments, the invention provides a process for producing a fucosylated oligosaccharide or a fucosylated antibody-glycan conjugate, comprising the steps of:
a. contacting glucose or fructose in the presence of an enzyme that converts the fructose or glucose to mannose;
b. contacting the mannose with an enzyme that converts mannose to mannose-6-phosphate;
c. contacting the mannose-6-phosphate with an enzyme that converts mannose-6-phosphate to mannose-1-phosphate;
d. contacting the mannose-1-phophate with an enzyme that converts the mannose-1-phophate to GDP-D-mannose;
e. contacting the GDP-D-mannose with an enzyme that converts GDP-D-mannose to GDP-4-keto-6-deoxymannose;
f. contacting the GDP-4-keto-6-deoxymannose with an enzyme that converts the GDP-4-keto-6-deoxymannose to GDP-L-fucose;
g. contacting the GDP-L-fucose with a disaccharide, an oligosaccharide or an antibody-glycan conjugate with an enzyme that fucosylates the disaccharide, oligosaccharide or the antibody-glycan conjugate; and
h. obtaining a fucosylated disaccharide, oligosaccharide or a fucosylated antibody-glycan conjugate.
wherein each of the enzymes is immobilized and wherein the process contains a set of regeneration enzyme systems to convert ADP to ATP, PPi to Pi, GDP to GTP, and NADP+ to NADPH.

In certain embodiments, the process comprises immobilized regeneration enzymes formate dehydrogenase, pyruvate oxidase, acetate kinase, catalase, and inorganic pyrophosphatase. In certain embodiments, the immobilized regeneration enzymes are phosphite dehydrogenase, pyruvate oxidase, acetate kinase, catalase, and inorganic pyrophosphatase. In certain embodiments, the immobilized regeneration enzymes are formate dehydrogenase, polyphosphate kinase, and inorganic pyrophosphatase. In certain embodiments, the immobilized regeneration enzymes are phosphite dehydrogenase, polyphosphate kinase, and inorganic pyrophosphatase.

In certain embodiments, the enzyme that converts the glucose to mannose is an immobilized D-mannose isomerase. In certain embodiments, the enzyme that converts the fructose to mannose is an immobilized N-acetyl-d-glucosamine-2-epimerase. In certain embodiments, the enzyme that converts mannose to mannose-6-phosphate is an immobilized hexokinase. In certain embodiments, the enzyme that converts mannose-6-phosphate to mannose-1-phosphate is an immobilized phosphomannomutase. In certain embodiments, the enzyme that converts mannose-1-phophate to GDP-D-mannose is an immobilized GDP-mannose pyrophosphorylase. In certain embodiments, the enzyme that converts GDP-D-Man to GDP-4-keto-6-deoxymannose is an immobilized GDP-mannose-4,6-dehydratase. In certain embodiments, the enzyme that converts GDP-4-keto-6-deoxymannose to GDP-L-fucose is an immobilized GDP-fucose synthase.

In some embodiments, the enzyme that fucosylates the disaccharide, oligosaccharide, or antibody-glycan conjugate is an immobilized fucosyl transferase. In some embodiments, the GDP-mannose is polymerized to mannans via transferase enzymes. In certain embodiments, the oligosaccharide is 3′SL, LNTII, LNT, LNnT, LNFPI, LNFPII, LNFPIII, LSTa, LSTb, LSTc, LSTd and DSLNT, LNnH, 3′″₃,3′″₆-di-O-α-Sia-LNnH 3′″₃,3′″₆-di-O-α-Sia-(3″₃,3″₆-di-O-α-Fuc)-LNnH, biantennary sialylated or fucosylated lacto-N-neohexaoses and neoheptaoses, α-2,3-sialyl lacto-N-neopentaose, linear fucosyl- and sialyl-lacto-N-neo-pentaoses, linear lacto-N-neopentaoses, or biantennary lacto-N-neohexaoses and heptaoses. In certain embodiments, the glycans are 3′SL LNTII, LNT, LNnT, LNFPI, LNFPII, LNFPIII, LSTa, LSTb, LSTc, LSTd, DSLNT, 2′-FL, LNnH, DSLNnH, or DSDFLNNH. In other embodiments, the glycans are 3′SL, LNTII, 2′-FL, LNFPI, LSTa. In certain embodiments, the oligosaccharide is lactose, LNT, LNnT, LNnH, or LNH. In certain embodiments, the fucosylated oligosaccharide is 2′FL or 3-FL. In certain embodiments, the d fucosylated oligosaccharide is a fucosylated HMO. In certain embodiments, the fucosylated oligosaccharide is fucosylated LDFT, fucosylated LNFP I, fucosylated LNFP II, fucosylated LNFP V, fucosylated LNDFH I, fucosylated LNDFH II, fucosylated DFLNH a, or fucosylated DFLNHc.

In certain embodiments, the process occurs in a single reaction vessel. In some embodiments, the invention provides a machine, comprising the immobilized enzymes of any one of the invention's embodiments wherein the machine produces a fucosylated disaccharide, fucosylated oligosaccharide, or fucosylated antibody-glycan conjugate. In certain embodiments, immobilized enzymes are contained within a single reaction vessel.

In certain embodiments, the pathway uses immobilized enzymes, such as enzymes that are immobilized within bionanocatalysts (BNCs) that in turn are embedded within scaffolds. Bionanocatalysts (BNCs) according to this invention comprise an enzyme self-assembled with magnetic nanoparticles (MNPs). The BNCs self-assemble with the scaffolds. In certain embodiments, each enzyme is immobilized within the BNC.

In certain embodiments, fucosylated human milk oligosaccharides (HMOs) are produced.

The one-pot integrated fucosylation synthesis may be done in batch or flow. It should be understood that modifying Examples 11-15 to be in-flow is within the scope of this invention. In a flow reactor, such as a packed bed reactor, the mixture of reagents is flowed through the flow reactor.

In certain embodiments, the scaffolded BNCs are inside of modular flow cells for flow manufacturing. In certain embodiments, the invention provides continuous flow processing where each step of synthesis is conducted in modules. In production mode, these modules contain full systems of enzymes—sugar activation and sugar transfer—for specifically building glycans. In some embodiments, the glycans are oligosaccharides.

In some embodiments, the scaffolds comprise magnetic metal oxides. In some embodiments, the scaffolds are high magnetism and high porosity composite blends of thermoplastics comprising magnetic particles that form powders. They may be single-layered or multiple-layered materials that hold the BNCs. Such designed objects may be produced using 3D printing by sintering composite magnetic powders. In some embodiments, Selective Laser Sintering (SLS) is used. The modular flow cells may be mixed and matched for a highly customizable and highly efficient manufacturing process. In preferred embodiments, human milk oligosaccharides (HMOs) are produced.

In certain some embodiments the immobilized enzymes are non-magnetic. In certain the immobilized enzymes do not comprise nanoparticles.

Thus, the invention provides cell-free productions of defined glycans with combinatorial bionanocatalysts (BNCs) organized in sequential modules.

### BRIEF DESCRIPTION OF THE DRAWINGS

FIG. 1A depicts a summary of galactosylation using sucrose to obtain LNT, LNnT, or lactose and a legend relevant to all figures herein (“2: Lac Synthase” should be “1d) Lac Synthase”). FIG. 1B depicts LNT obtained from LNTII. FIG. 1C depicts LNnT obtained from LNTII. FIG. 1D depicts LNT obtained from lactose. FIG. 1E depicts lactose obtained from glucose. FIG. 1F depicts GOS obtained from galactose and sucrose.

FIGS. 2A-2D depict sialylations with sialoside and lactose hydrolysis. FIG. 2A depicts the route for obtaining LSTa from LNT. FIG. 2B depicts the route for obtaining LSTc from LNnT. FIG. 2C depicts the route for obtaining LSTd from LNnT. FIG. 2D depicts the route for obtaining DSLNT from LNT.

FIGS. 3A-3C depict fucosylation using fucoside and lactose hydrolysis. FIG. 3A depicts the route for obtaining LNFPI from LNT. FIG. 3B depicts the route for obtaining LNFPII from LNT. FIG. 3C depicts the route for obtaining LNFPII from LNnT.

FIG. 4 depicts the addition of N-Acetylglucosamine to prepare LNTII from lactose and chitin.

FIG. 5 depicts lactose and its functional derivatives.

FIG. 6 depicts a general embodiment of this invention and an overview of the methods employed in this invention.

FIG. 7 depicts a general embodiment and methods of this invention employing an auxiliary enzyme.

FIG. 8 depicts a legend depicting the symbols used in FIGS. 9-12.

FIG. 9A depicts a summary of galactosylation using sucrose to obtain LNT, LNnT, or lactose.

FIG. 9B depicts a route to LNT obtained from LNTII. FIG. 9C depicts a route to LNnT obtained from LNTII. FIG. 9D depicts a route to LNT obtained from lactose. FIG. 9E depicts a route to lactose obtained from glucose. FIG. 9F and FIG. 9G depict routes to obtain lactose from glucose. FIG. 9H depicts a route to Galactooligosaccharides obtained from galactose and sucrose.

FIGS. 10A-10D depict sialylations with sialoside and lactose hydrolysis. FIG. 10A depicts the route for obtaining LSTa from LNT. FIG. 10B depicts the route for obtaining LSTc from LNnT. FIG. 10C depicts the route for obtaining LSTd from LNnT. FIG. 10D depicts the route for obtaining DSLNT from LNT.

FIGS. 11A-11C depict fucosylation using fucoside and lactose hydrolysis. FIG. 11A depicts the route for obtaining LNFPI from LNT. FIG. 11B depicts the route for obtaining LNFPII from LNT. FIG. 11C depicts a route for obtaining LNFPII from LNnT. FIG. 11D depicts the route for obtaining DFL from 2′-FL. FIG. 11E depicts a route for obtaining 3-FL from 2′-FL.

FIG. 12A depicts N-acetylglucosaminylation using lacto-N-biose as a GlcNAc donor. FIG. 12B depicts a branched HMO from Lacto-N-biose (LNB) and LNnT.

FIG. 13 depicts a general embodiment of this invention and an overview of the methods employed in this invention that provide a processing enzyme to convert a principal product to a principal product derivative.

FIG. 14 depicts a general embodiment and methods of this invention employing an auxiliary enzyme and a Processing Enzyme to convert a Principal Product to a Principal Product Derivative.

FIGS. 15-18 relate to the integrated fucosylation pathway provided by this invention.

FIG. 15 depicts the in situ GDP-L-fucose production for 2′-FL synthesis or other fucosylation reactions (Pyruvate-AcK/PyrOx+FDH regeneration). See Example 12.

FIG. 16 depicts the in situ GDP-L-fucose production for 2′-FL synthesis or other fucosylation reactions (Pyruvate-AcK/PyrOx+PtxD regeneration). See Example 13.

FIG. 17 depicts the in situ GDP-L-fucose production for 2′-FL synthesis or other fucosylation reactions (PolyP-PPK+FDH regeneration). See Example 14.

FIG. 18 depicts the in situ GDP-L-fucose production for 2′-FL synthesis or other fucosylation reactions (PolyP-PPK+PtxD regeneration). See Example 15.

### DETAILED DESCRIPTION OF THE INVENTION

The present invention provides integrated streamlined pathways using enzymes and activated sugars to obtain sugar compounds, oligosaccharides, and their derivatives. In certain embodiments the oligosaccharides are complex oligosaccharides, including glycans 4 sugar units or larger, and including glycans.

In one aspect, the invention provides cell-free de-novo synthesis of glycans employing in situ generated activated sugar donors and catalytic amounts of nucleotides. Integrated pathways of enzymes lead to glycosylated (sugar-containing compounds) including, but not limited to, saccharides, oligosaccharides, or antibody-glycan conjugates starting from sugars, including, but not limited to, plant sugars. The benefits of the systems of reactants and enzymes of this aspect of the invention comprise, consist essentially of, or consist of:
Avoiding enzymes that recycle cofactors.
Avoiding nucleotides in stoichiometric or excess quantities relative to either the sugar donor precursor or the acceptor or both the sugar donor precursor and the acceptor.
Lower cost by using a nucleotide starting material compared to using a nucleotide-sugar as a starting material.
Ability to use nucleotides at less than 0.1 molar equivalents (<10%) compared to acceptor molecule or limiting reagent.
Ability to use nucleotides at less than 0.1 molar equivalents (<50%) compared to sugar donor molecule or limiting reagent.
Avoiding nucleotide triphosphates
Avoiding inorganic phosphate byproducts
Achieving high reaction conversions via the hydrolysis of the high energy sugar donor precursor into a product that is in some embodiments enzymatically processed by processing enzymes for enhanced conversion.
Avoiding enzyme inhibition by organic phosphates.
Avoiding enzyme inactivation and reaction inhibition by using magnesium (Mg) rather than manganese (Mn).
Requiring relatively low molar sucrose concentrations equivalents concentrations (1-4 eq.) relative to acceptor.

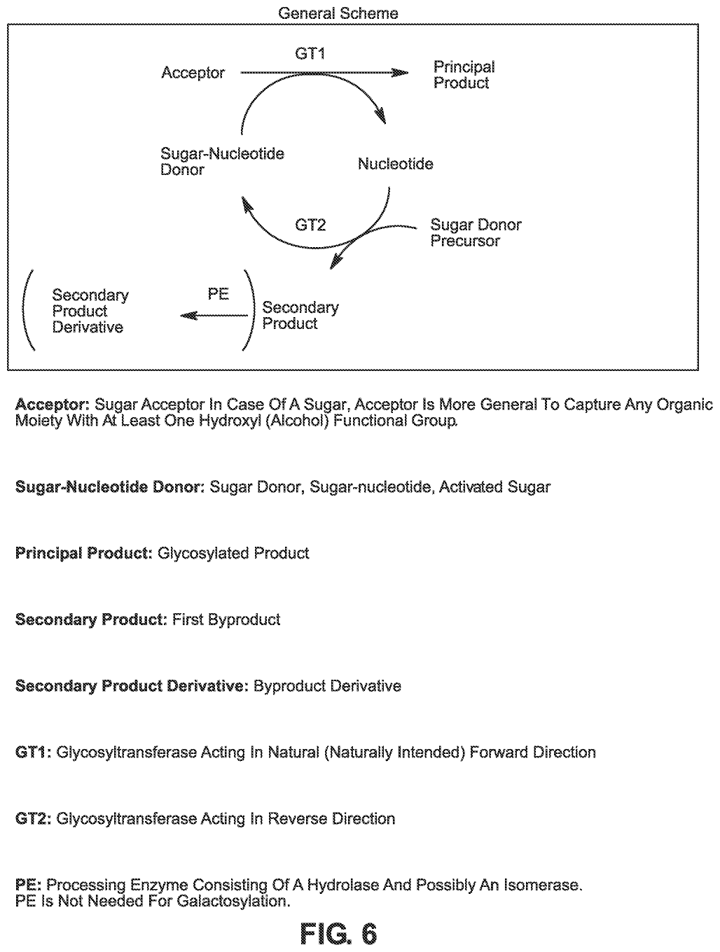

FIG. 6 depicts a general embodiment of this invention and an overview of the methods employed in this invention. As used herein, an “acceptor” is the moiety that accepts transfer of a sugar unit from a sugar-nucleotide donor. A transferase, designated here as GT1, catalyzes the sugar transfer thereby producing a “principal product” that is the glycosylated product provided by this invention. The sugar-nucleotide donor is a compound comprising a sugar unit and a nucleotide obtained from a reaction of a sugar donor precursor and a nucleotide in the presence of a transferase, here designated at GT2. GT2 is acting in the reverse direction relative to GT1. GT1 transfers a sugar unit to an acceptor form a sugar-acceptor compound. GT2 acts to remove a sugar unit. The nucleotide is present in less than stoichiometric amounts and therefore the sugar-nucleotide donor is regenerated and used in another reaction cycle. The reaction of a sugar donor precursor and a nucleotide in the presence of a transferase produces a secondary product in addition to the sugar-nucleotide donor. The breakdown of the sugar donor precursor into smaller sugar units is an energy source for the methods of this invention. The secondary product may be transformed into a secondary product derivative in the presence of a processing enzyme.

Accordingly, in one embodiment, this invention provides a method for obtaining a glycosylated compound (herein, “glycosylated principal product” by combining in a reaction vessel the following: a sugar donor precursor, an acceptor, a transferase, and a catalytic amount of a nucleotide. The method involves iterative reaction cycles to produce a glycosylated principal product and a secondary product. Without being bound by theory, once combined in a reaction mixture, the nucleotide reacts with the sugar donor precursor in the presence of a transferase to form a sugar-nucleotide donor. The sugar of the sugar-nucleotide donor is transferred to an acceptor to provide the glycosylated compound, a secondary product, and a regenerated nucleotide. As the nucleotide is present in catalytic quantities, the sugar-nucleotide donor will be formed in about catalytic quantities. The reaction then cycles through additional iterations of the reaction.

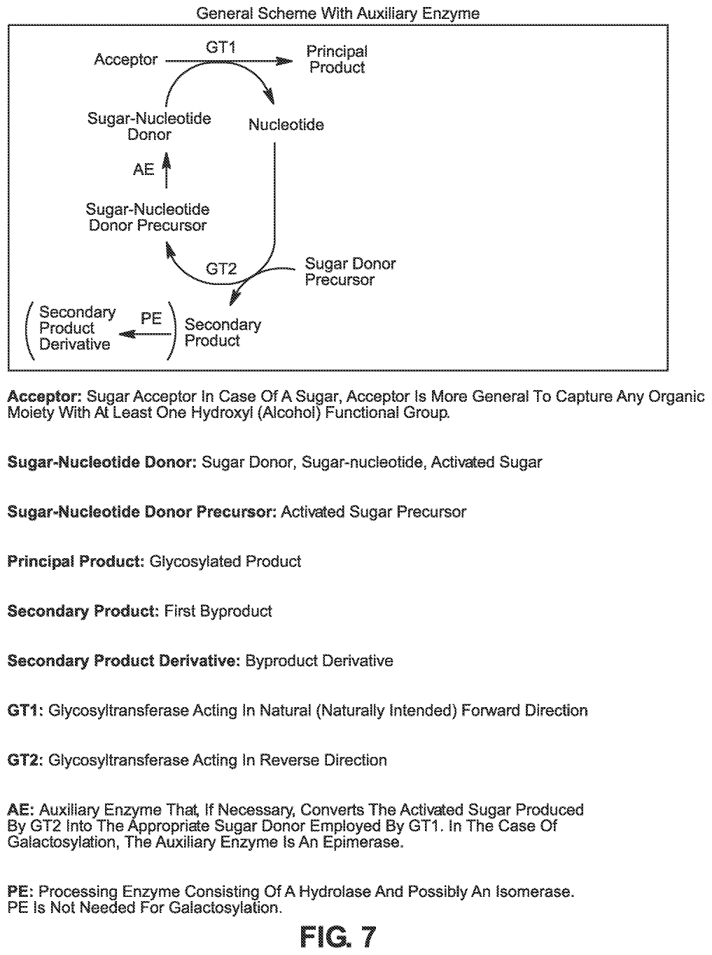

FIG. 7 depicts a general embodiment and methods of this invention employing a sugar-nucleotide donor precursor. As depicted the product of the reaction between a nucleotide and a sugar donor precursor in the presence of a transferase provides a sugar-nucleotide donor precursor that undergoes a further reaction in the presence of an auxiliary enzyme to obtain the sugar-nucleotide donor employed in the methods of this invention.

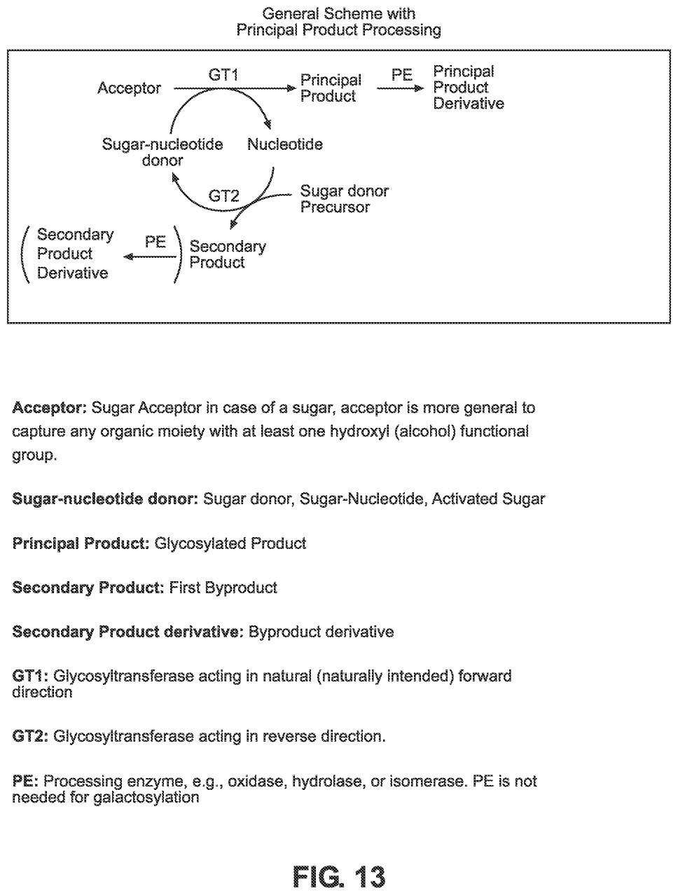

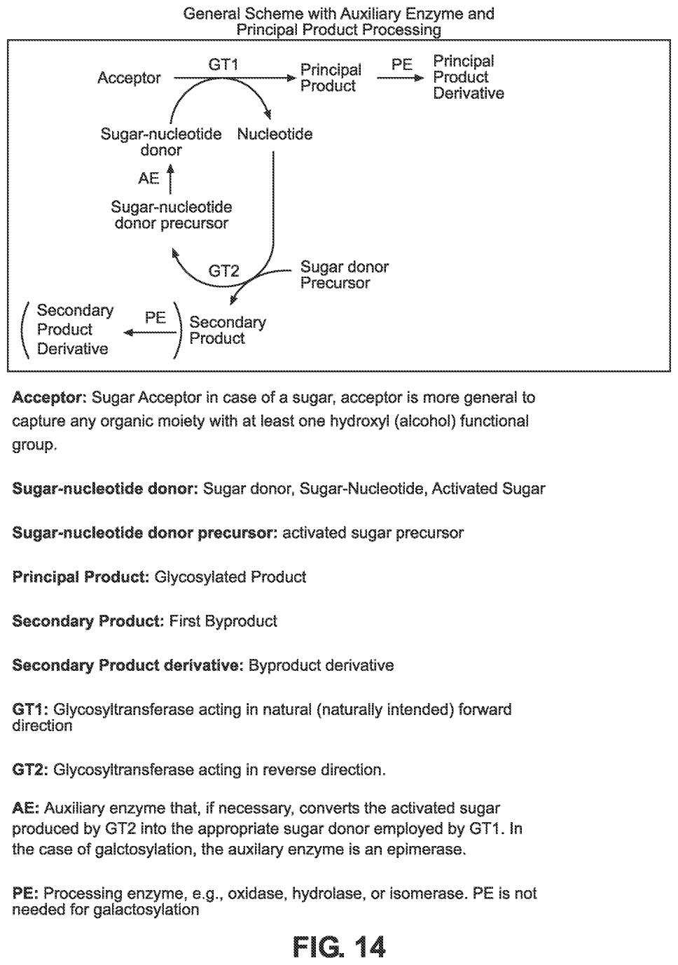

FIG. 13 and FIG. 14 depict general embodiments of this invention that employ a processing enzyme to convert a principal product to a principal product derivative. In Example 10, LSTb is obtained from LNT by employing this method. DSLNT is the Principal Product that is converted to LSTb by the neuraminidase.

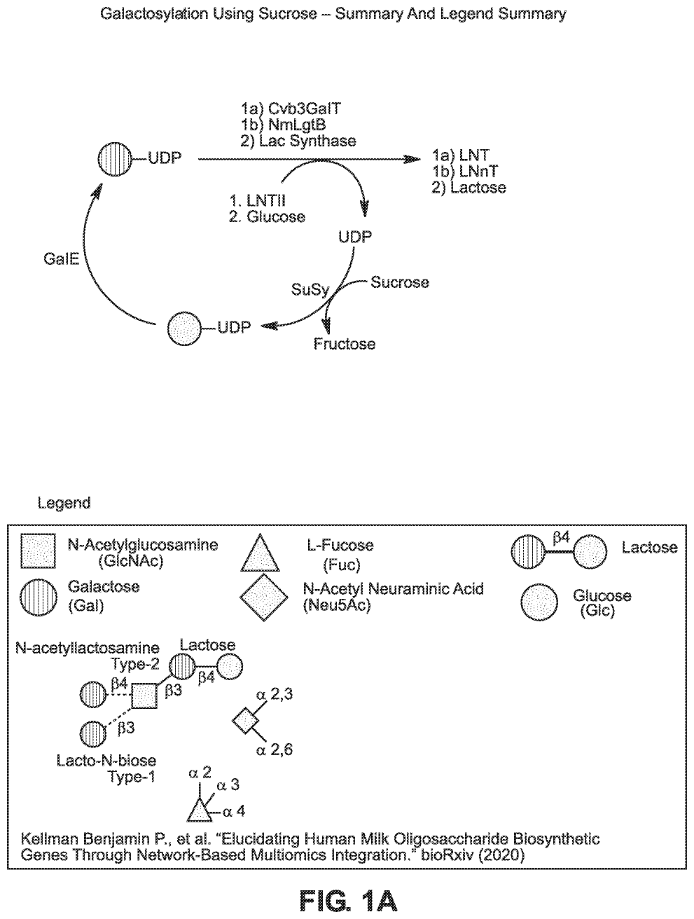

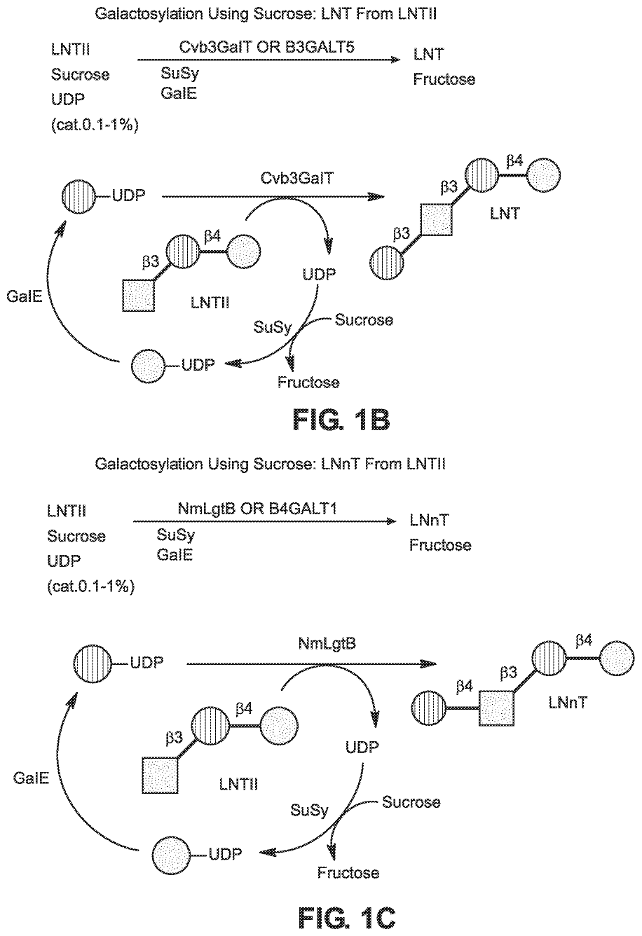

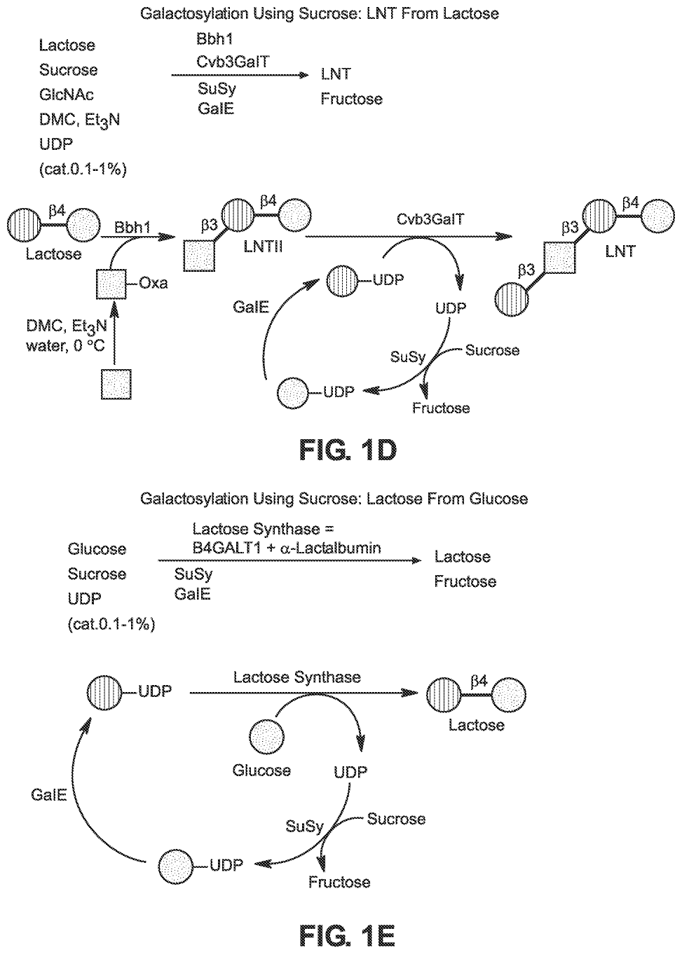

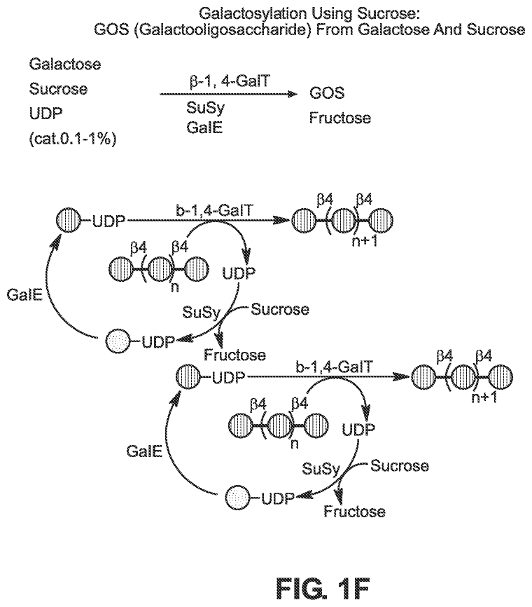

FIGS. 1A-1F depict a summary of galactosylation reactions using sucrose and a legend. LNTII and glucose are acceptor molecules that are galactosylated to provide LNT, LNnT, or lactose depending on the conditions (see (FIG. 1B-1D).

FIG. 1B depicts a route for adding galactose to LNTII to obtain LNT. UDP-glucose and fructose are obtained from sucrose in the presence of sucrose synthase and UDP. The UDP-galactose is obtained from UDP-glucose in the presence of GalE. Galactose from UDP-galactose is transferred to the LNTII in the presence of a transferase that forms a β 3 type linkage (as depicted, Cvb3GalT or B3GALT5). See Example 2a A.

FIG. 1C depicts a route for adding galactose to LNTII to obtain LNnT. Formation of UDP-galactose and fructose follows the same route as in FIG. 1B. In this scheme however a transferase that forms a β 4 type linkage (as depicted NmLgtB or B4LAGT1) is present. Galactose is transferred to LNTII to provide LNnT. See Example 2a B.

FIG. 1D depicts a route for obtaining LNT from lactose. N-acetylglucosamine is converted into an oxazoline in the presence of DMC, triethylamine, and water. N-acetylglucosamine is transferred to lactose via a β3 linkage to provide LNTII. UDP-ga FIGS. 1A-1F depict a summary of galactosylation reactions using sucrose and a legend. LNTII and glucose are acceptor molecules that are galactosylated to provide LNT, LNnT, or lactose depending on the conditions (see (FIG. 1B-1D).

FIG. 1B depicts a route for adding galactose to LNTII to obtain LNT. UDP-glucose and fructose are obtained from sucrose in the presence of sucrose synthase and UDP. The UDP-galactose is obtained from UDP-glucose in the presence of GalE. Galactose from UDP-galactose is transferred to the LNTII in the presence of a transferase that forms a β 3 type linkage (as depicted, Cvb3GalT or B3GALT5). See Example 2a A.

FIG. 1C depicts a route for adding galactose to LNTII to obtain LNnT. Formation of UDP-galactose and fructose follows the same route as in FIG. 1B. In this scheme however a transferase that forms a β 4 type linkage (as depicted NmLgtB or B4LAGT1) is present. Galactose is transferred to LNTII to provide LNnT. See Example 2a B.

FIG. 1D depicts a route for obtaining LNT from lactose. N-acetylglucosamine is converted into an oxazoline in the presence of DMC, triethylamine, and water. N-acetylglucosamine is transferred to lactose via a (33 linkage to provide LNTII. UDP-galactose and fructose are obtained as described in FIG. Ba. In the presence of a transferase (as depicted Cvb3GalT), galactose is transferred to the acceptor LNTII to provide LNT.

FIG. 1E depicts lactose obtained from glucose. UDP-galactose is obtained as described in FIG. 1B. Galactose is transferred to glucose in the presence of lactose synthase to obtain lactose. See Example 2a D.

FIG. 1F depicts GOS obtained from galactose and sucrose. UDP-galactose and fructose are obtained as described in FIG. 1B. The galactose is transferred to a galactooligosaccharide (wherein n is 3-15) to increase the galactose chain by 1. UDP-galactose is obtained as described in FIG. 1B. See Example 2a E.

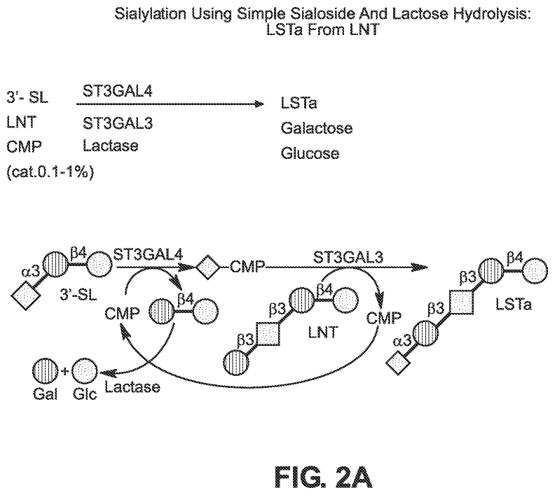

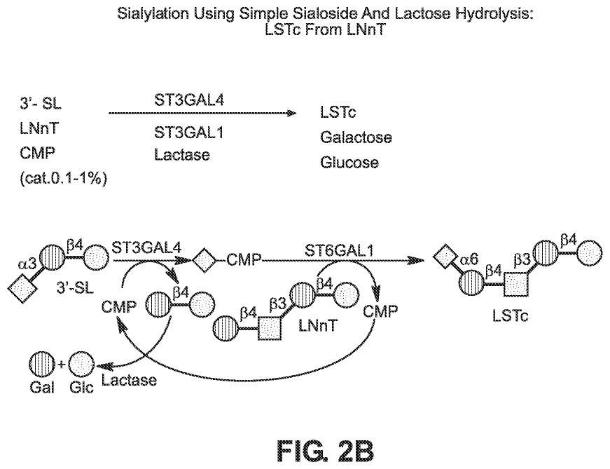

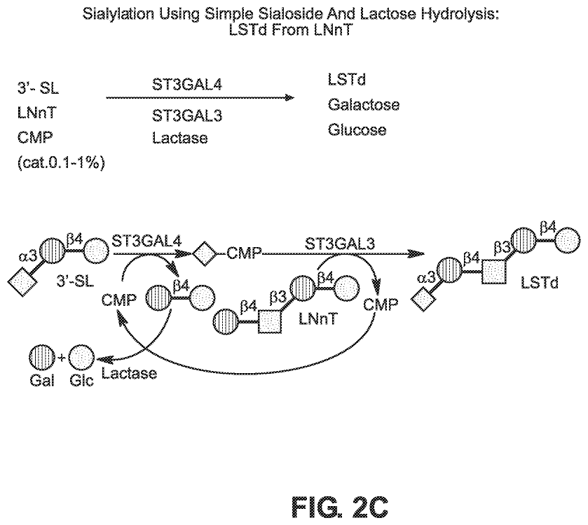

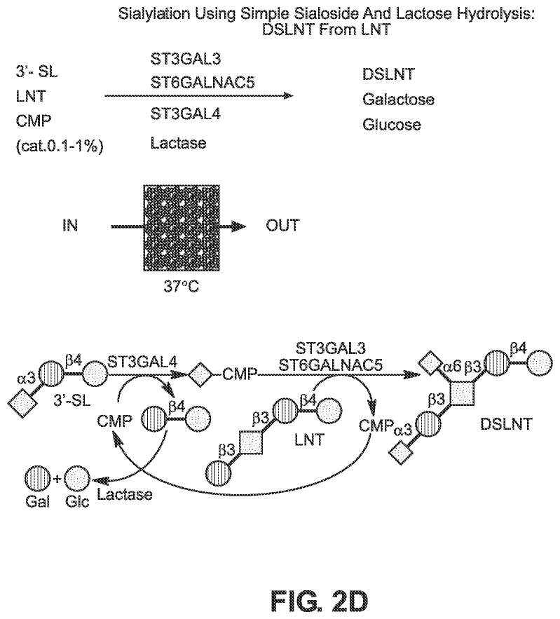

FIGS. 2A-2D depict sialylations with sialoside and lactose hydrolysis. CMP-N-acetyl neuraminic acid (Neu5Ac) is the nucleotide-sugar employed in each of FIGS. 2A-2C. As depicted in each figure, lactose is converted to galactose and glucose in the presence of lactase. CMP-Neu5Ac is obtained in the presence of 3′-SL and the transferase ST3GAL4.

FIG. 2A depicts a route for obtaining LSTa is from LNT. In the presence of the transferase ST3GAL3, N-acetyl neuraminic acid is transferred from CMP-Neu5Ac to LNT to obtain LSTc.

FIG. 2B depicts a route for obtaining LSTc from LNnT. In the presence the transferase ST6GAL1, N-acetyl neuraminic acid is transferred from CMP-Neu5Ac to LNnT to obtain LSTc.

FIG. 2C depicts a route LSTd is obtained from LNnT. In the presence the transferase ST3GAL3, N-acetyl neuraminic acid is transferred from CMP-Neu5Ac to LNnT to obtain LSTd.

In FIG. 2D DSLNT is obtained from LNT. In the presence the transferase ST6GAL3 and ST6GALNAC5, N-acetyl neuraminic acid is transferred from CMP-Neu5Ac to LNT to obtain DSLNT.

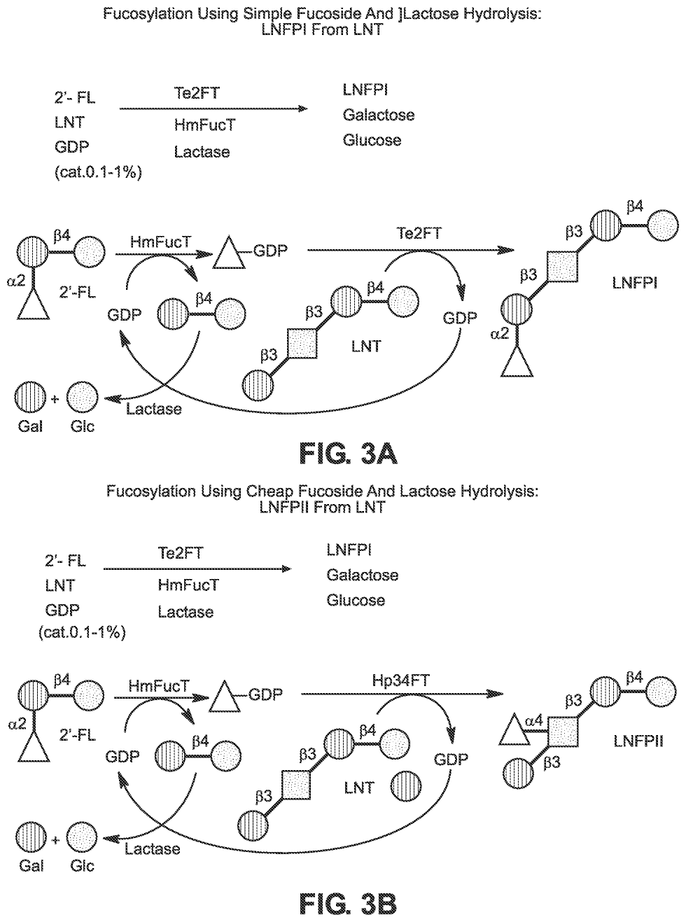

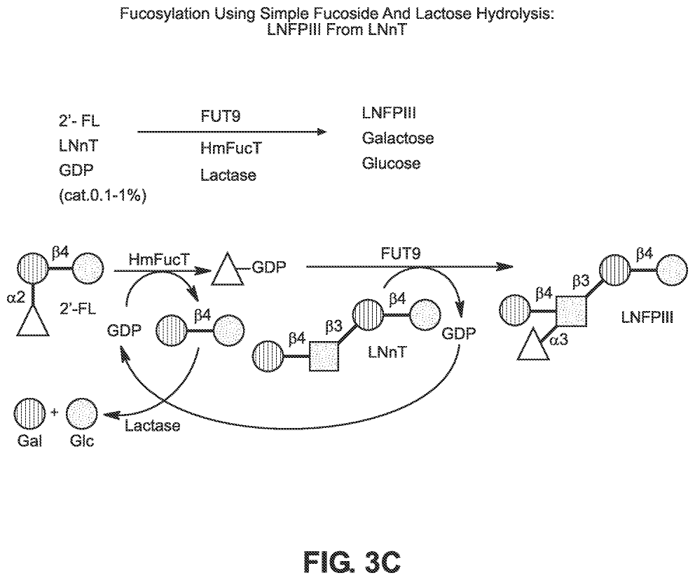

FIGS. 3A-3C depicts fucosylation using fucoside and lactose hydrolysis. GDP-1-fucose is the nucleotide-sugar used in each of FIG. 3a-3c. 2′-FL in the presence of a transferase (HmFucT), lactose, lactase, and GDP provides GDP-1-fucose (GDP-Fuc), galactose, and glucose.

FIG. 3A depicts a route for obtaining LNFPI from LNT. GDP-Fuc in the presence of LNT and Te2FT provides LNFPI.

FIG. 3B depicts a route for obtaining LNFPII from LNT. GDP-Fuc in the presence of LNT and a transferase (Hp34FT) provides LNFPII.

FIG. 3C depicts a route for obtaining LNFPIII from LNnT. GDP-Fuc in the presence of LNnT and a transferase (FUT9) provides LNFPIII.

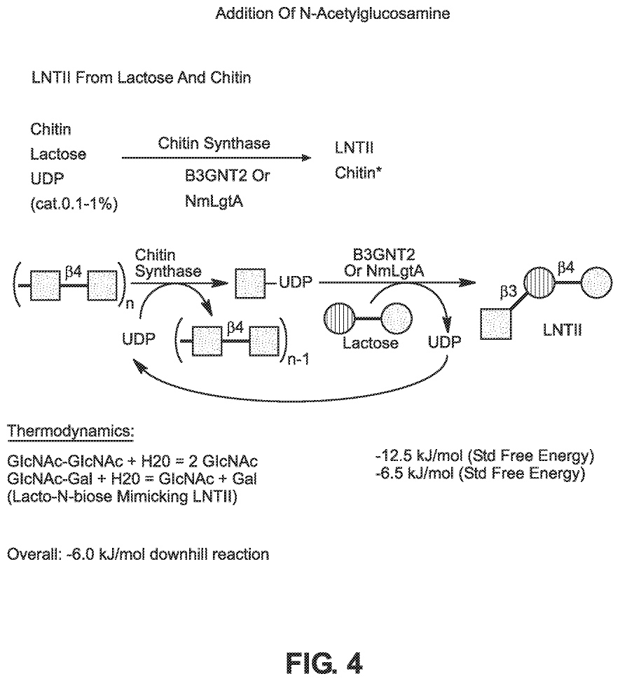

FIG. 4 depicts a route for adding N-acetylglucosamine to prepare LNTII from lactose and chitin. UDP-N-Acetylglucosamine (UDP-GlcNAc) is prepared from UDP, lactose, chitin in the presence chitin synthase and the presence of a transferase. (B3GNT2 or NmLgtA) and the GlcNAc is transferred to lactose to obtain LNTII. In the presence of a transferase (as depicted Cvb3GalT), galactose is transferred to the acceptor LNTII to provide LNT.

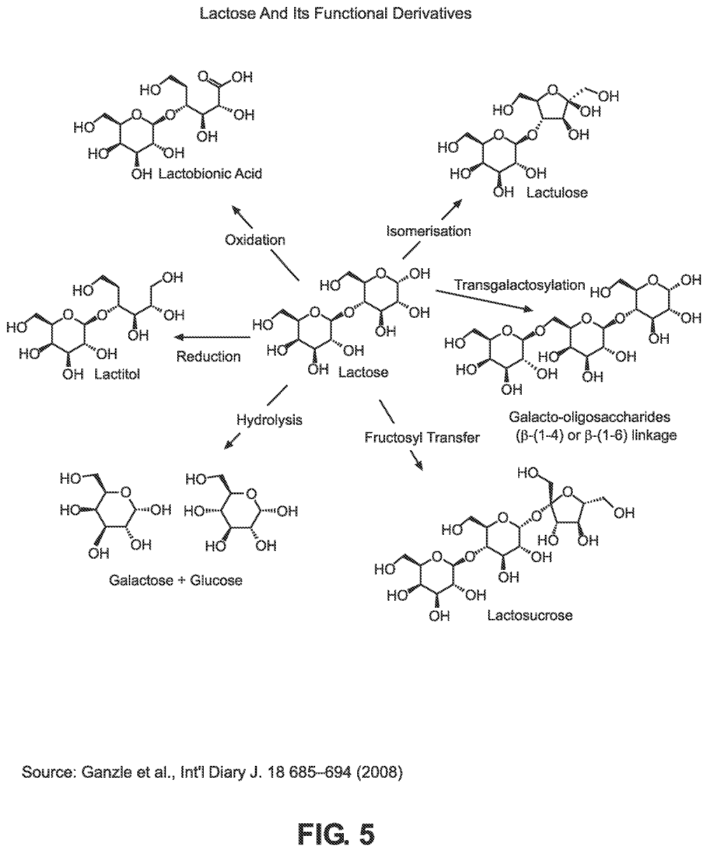

FIG. 5 depicts lactose and its functional derivatives.

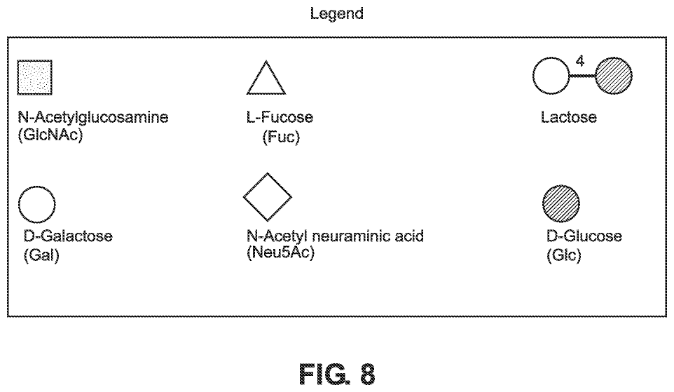

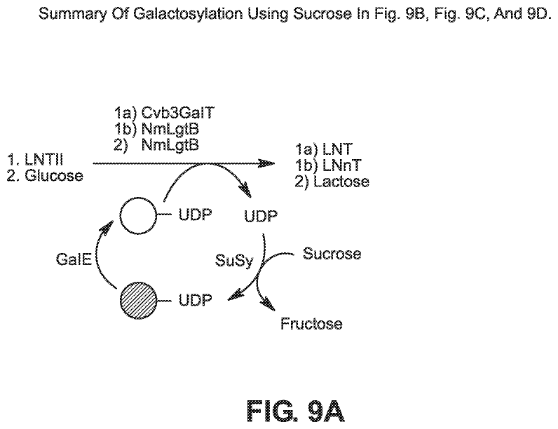

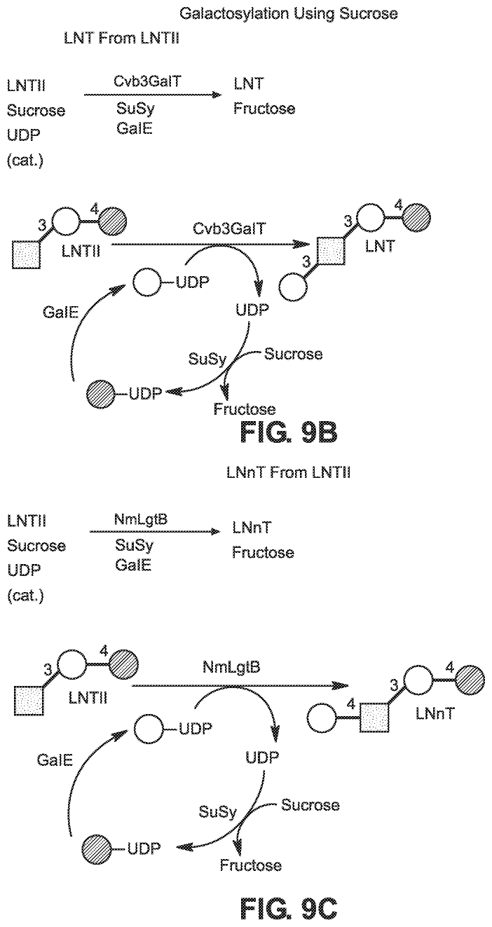

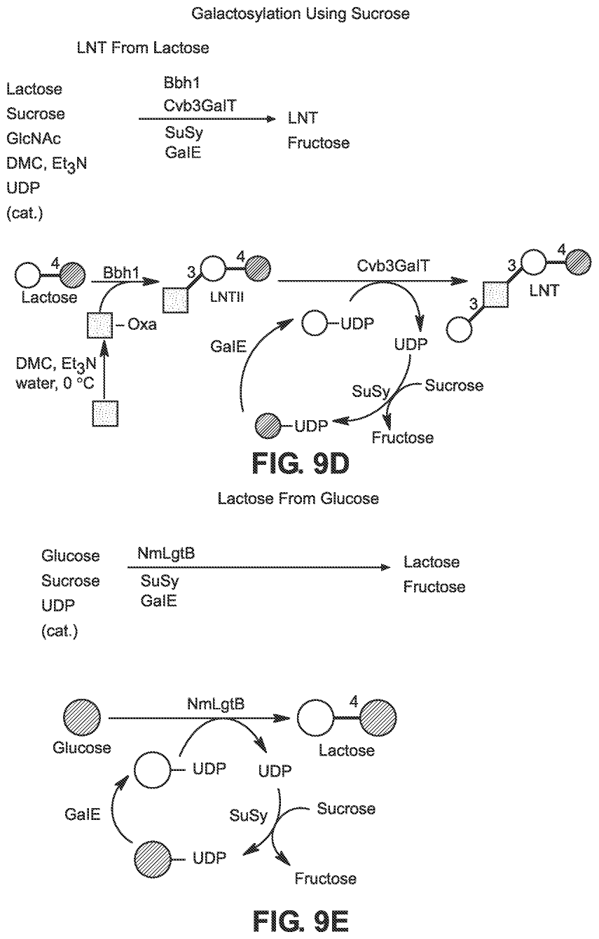

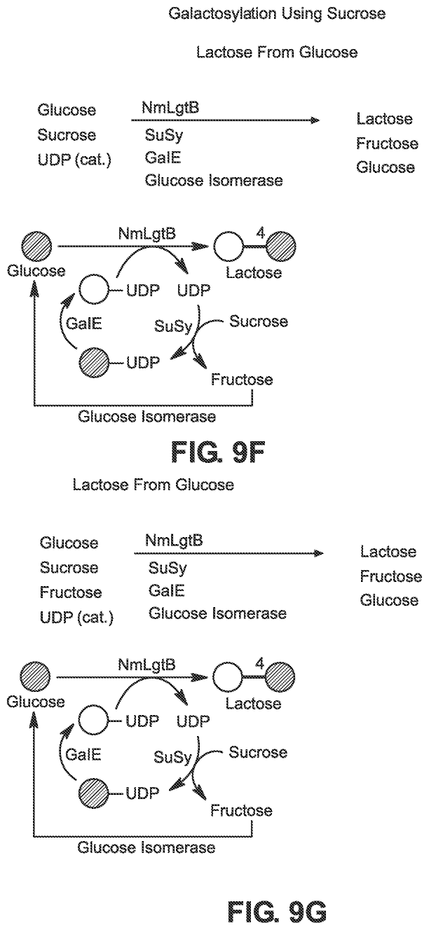

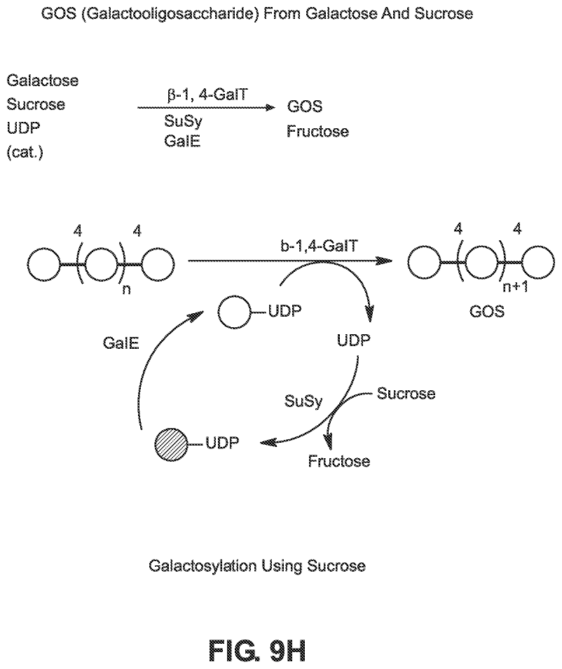

FIG. 8 depicts a legend for FIGS. 9-12. N-acetylglucosamine (GlcNAc) is depicted by a solid black square. L-fucose (fuc) is depicted by a solid white triangle. Lactose is depicted by a solid white circle linked by a line to a hatched circle, with a 4 indicating bonding. D-galactose (Gal) is depicted by a solid circle. N-acetyl neuraminic acid (Neu5Ac) is depicted as a solid white diamond. D-glucose is depicted by a hatched circle.

FIGS. 9A-9H depict a summary of galactosylation reactions using sucrose. LNTII and glucose are acceptor molecules that are galactosylated to provide LNT, LNnT, or lactose depending on the conditions (see (FIG. 9B-9D).

FIG. 9B depicts a route for adding galactose to LNTII to obtain LNT. UDP-glucose and fructose are obtained from sucrose in the presence of sucrose synthase and UDP. The UDP-galactose is obtained from UDP-glucose in the presence of GalE.

Galactose from UDP-galactose is transferred to the LNTII in the presence of a transferase that forms a β 3 type linkage (as depicted, Cvb3GalT). See Example 6A.

FIG. 9C depicts a route for adding galactose to LNTII to obtain LNnT. Formation of UDP-galactose and fructose follows the same route as in FIG. 9B. In this scheme however a transferase that forms a β 4 type linkage (as depicted NmLgtB) is present. Galactose is transferred to LNTII to provide LNnT. See Example 6B.

FIG. 9D depicts a route for obtaining LNT from lactose. N-acetylglucosamine is converted into an oxazoline in the presence of DMC, triethylamine, and water. N-acetylglucosamine is transferred to lactose via a (33 linkage in the presence of an aminidase (here Bbh1) to provide LNTII. UDP-galactose and fructose are obtained as described in FIG. 9B. In the presence of a transferase (as depicted Cvb3GalT), galactose is transferred to the acceptor LNTII to provide LNT.

FIG. 9E depicts lactose obtained from glucose. UDP-galactose is obtained as described in FIG. 9B. Galactose is transferred to glucose in the presence of lactose synthase to obtain lactose. See Example 6C.

FIG. 9F depicts lactose obtained from glucose, sucrose, and catalytic amounts of UDP using the four enzymes NmLgtB, AtSuSy1, GalE and Glucose (xylose) isomerase (EC 5.3. 1.5, D-xylose aldose-ketose-isomerase). See Example 7.

FIG. 9G depicts lactose obtained from glucose, sucrose, fructose and catalytic amounts of UDP using the four enzymes NmLgtB, AtSuSy1, GalE and Glucose (xylose) isomerase (EC 5.3. 1.5, D-xylose aldose-ketose-isomerase). See Example 8.

FIG. 9H depicts GOS obtained from galactose and sucrose. UDP-galactose and fructose are obtained as described in FIG. 9B. The galactose is transferred to a galactooligosaccharide (wherein n is 3-15) to increase the galactose chain by 1.

In some embodiments, the transferase is a galactosyltransferase that catalyzes the transfer of activated UDP-Galactose (UDP-Gal) to Glucose, Galactose or GlcNAc.

In certain embodiments, the galactosyltransferase is a β-1,3-galactosyltransferase (EC 2.4.1.122). In particular embodiments, the β-1,3-galactosyltransferase is Cvβ3GalT from Chromobacterium violaceum, WbgO from Escherichia coli, CgtB from Campylobacter jejuni, B3GALT1 from Homo sapiens, B3GALT2 from Homo sapiens, B3GALT4 from Homo sapiens, or B3GALT5 from Homo sapiens.

In certain embodiments, the galactosyltransferase is β-1,4-galactosyltransferase (EC 2.4.1.22, EC 2.4.1.38, EC 2.4.1.133, EC 2.4.1.275). In particular embodiments, the β-1,4-galactosyltransferase is NmLgtB from Neisseria meningitidis, NmLgtB-StGalE from Neisseria meningitidis and Streptococcus thermophilus, HpLgtB from Helicobacter pylori, B4GALT1 from Homo sapiens, B4GALT2 from Homo sapiens, B4GALT3 from Homo sapiens, B4GALT4 from Homo sapiens, B4GALT5 from Homo sapiens, B4GALT6 from Homo sapiens, B4GALT7 from Homo sapiens.

In certain embodiments, the galactosyltransferase is a β-1,6-galactosyltransferase. In a particular embodiment, the β-1,6-galactosyltransferase is GalT29A from Arabidopsis thaliana.

In certain embodiments, the galactosyltransferase is a α-1,3-galactosyltransferase (EC 2.4.1.87, EC 2.4.1.37). In particular embodiments, the α-1,3-galactosyltransferase is GTB (human proteins) synthetic gene expressed in E. coli or WbnL from E. coli.

In certain embodiments, the galactosyltransferase is a α-1,6-galactosyltransferase (EC 2.4.1.241).

In certain embodiments, the galactosyltransferase is selected from the galactosyltransferases depicted in the Figures.

In certain embodiments, an auxiliary enzyme is employed in a route from a sugar donor precursor to a sugar-nucleotide donor. In those embodiments employing an auxiliary enzyme, a sugar donor precursor is converted to a sugar-nucleotide donor precursor in the presence of a transferase. The sugar-nucleotide donor precursor is then converted a sugar-nucleotide donor in the presence of the auxiliary enzyme. In certain embodiments, the auxiliary enzyme is a UDP-glucose 4-epimerase. In the galactosylation routes depicted in FIG. 9A-H, the auxiliary enzyme is a UDP-glucose 4-epimerase from Bifidobacterium longum.

In some embodiments, an auxiliary enzyme is an oxidase (UDP-Glc-6-dehydrogenase, EC 1.1.1.22) that catalyzes the conversion of UDP-Glc to UDP-GlcA (UDP-Glucuronic acid).

In some embodiments, an auxiliary enzyme is an enzyme that isomerize activated sugar nucleotides. In certain embodiments, the enzyme that isomerizes activated sugar nucleotides is an epimerase (UDP-Gal-4-epimerase, EC 5.1.3.2) that catalyzes the conversion of UDP-Glc to UDP-Gal. In a particular embodiment, the epimerase is EcGalE from Escherichia coli. In another particular embodiments, the epimerase is StGalE from Streptococcus thermophilus.

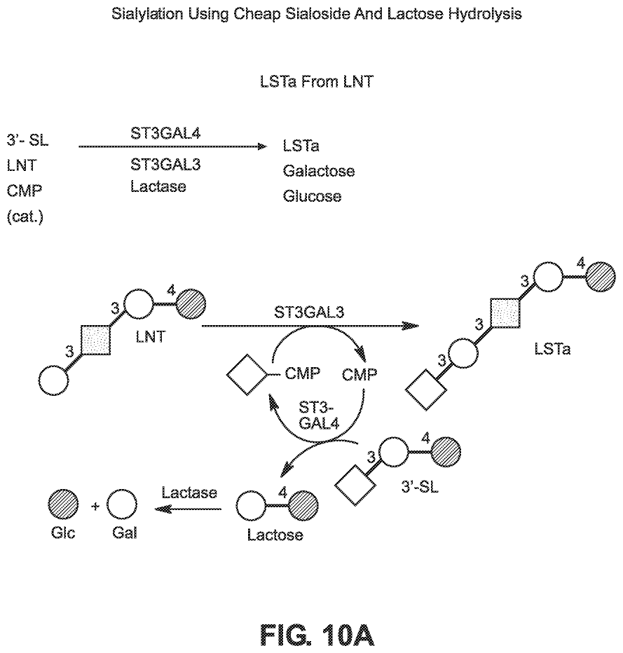

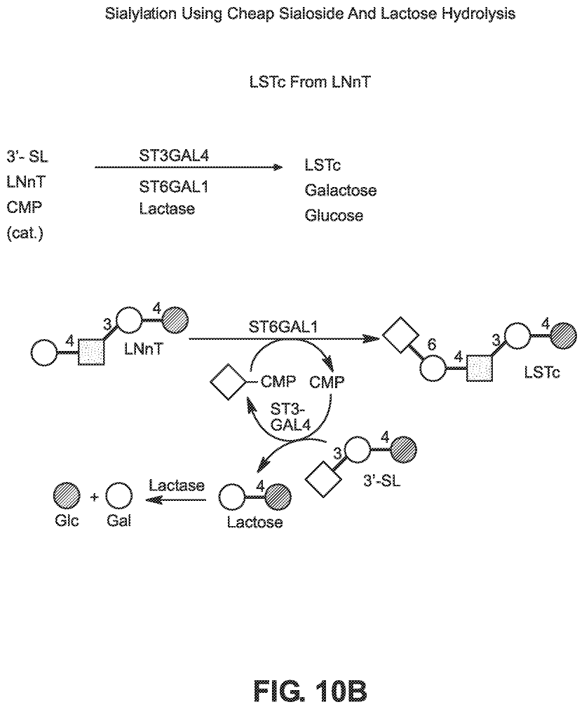

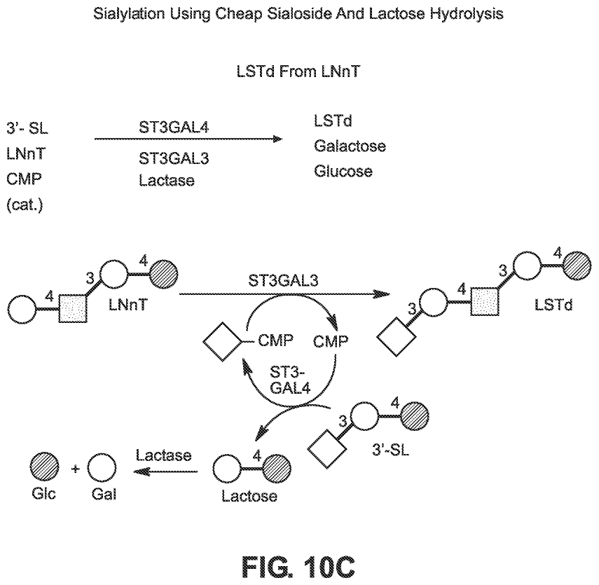

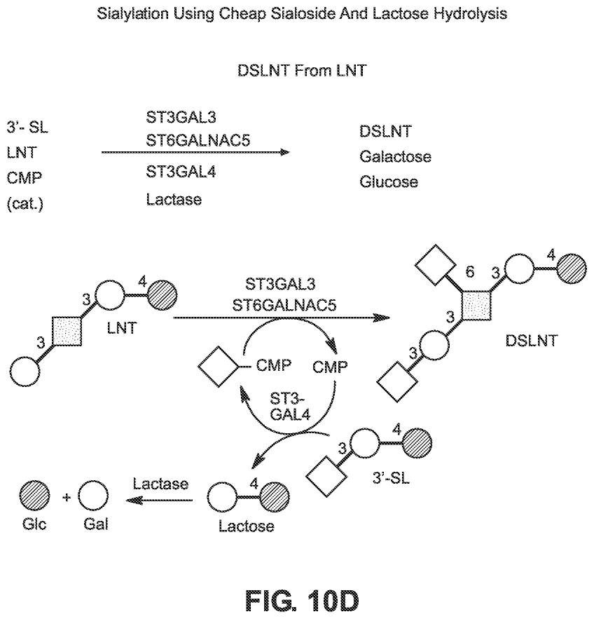

FIGS. 10A-10D depict sialylations with low cost sialoside and lactose hydrolysis. CMP-N-acetyl neuraminic acid (Neu5Ac) is the nucleotide-sugar employed in each of FIGS. 10A-10D. As depicted in each figure, lactose is converted to galactose and glucose in the presence of lactase, and CMP-Neu5Ac is obtained in the presence of 3′-SL and the transferase ST3GAL4.

FIG. 10A depicts a route for obtaining LSTa from LNT. In the presence of the transferase ST3GAL4, ST3GAL3, and lactase, N-acetyl neuraminic acid is transferred from CMP-Neu5Ac to LNT to obtain LSTa.

FIG. 10B depicts a route for obtaining LSTc from LNnT. In the presence the transferase ST3GAL4, ST6GAL1, and lactase, N-acetyl neuraminic acid is transferred from CMP-Neu5Ac to LNnT to obtain LSTc.

FIG. 10C depicts a route LSTd is obtained from LNnT. In the presence the transferase ST3GAL4, ST3GAL3, and lactase N-acetyl neuraminic acid is transferred from CMP-Neu5Ac to LNnT to obtain LSTd.

In FIG. 10D depicts a route for obtaining DSLNT from LNT. In the presence the transferase ST3GAL3, ST6GALNAC5, ST3GAL4, and lactase, N-acetyl neuraminic acid is transferred from CMP-Neu5Ac to LNT to obtain DSLNT.

Some embodiments of this invention employ sialyltransferase to catalyze the transfer of CMP-sialic acid (e.g., CMP-Neu5Ac) onto either GlcNAc, Galactose or Neu5Ac. The methods depicted in FIG. 2A-2D and FIG. 10A-D employ sialyltransferases to transfer a sialyl group from a sugar nucleotide donor to an acceptor.

In certain embodiments of this invention the sialyltransferase is an α-2,3-sialyltransferase (EC 2.4.99.4, EC 2.4.99.6, EC 2.4.99.7, EC 2.4.99.9). In particular embodiments, the α-2,3-sialyltransferase is PmST1 (wild type and mutants) from Pasteurella multocida, NmST1-NmCSS fusion from Neisseria meningitidis, ST3GAL1 from Homo sapiens, ST3GAL2 from Homo sapiens, ST3GAL3 from Homo sapiens, ST3GAL4 from Homo sapiens, or ST3GAL5 from Homo sapiens, ST3GAL6 from Homo sapiens.

In certain embodiment of this invention the sialyltransferase is an α-2,6-sialyltransferase (EC 2.4.99.1, EC 2.4.99.3). In particular embodiments, the α-2,6-sialyltransferase is Pd26ST from Photobacterium damsel, ST6GAL1 from Homo sapiens, ST6GAL2 from Homo sapiens, ST6GALNAC1 from Homo sapiens, ST6GALNAC2 from Homo sapiens, ST6GALNAC3 from Homo sapiens, ST6GALNAC4 from Homo sapiens, ST6GALNAC5 from Homo sapiens, or ST6GALNAC6 from Homo sapiens.

In certain embodiments of this invention, the sialyltransferase α-2,8-sialyltransferase (EC 2.4.99.8). In particular embodiments, the α-2,8-sialyltransferase is α-2,3/8-sialyltransferase from Campylobacter jejuni, ST8SIA1 from Homo sapiens, ST8SIA2 from Homo sapiens, ST8SIA3 from Homo sapiens, ST8SIA4 from Homo sapiens, or ST8SIA5 from Homo sapiens.

In certain embodiments, the sialyltransferae is selected from the sialylltransferases depicted in the Figures.

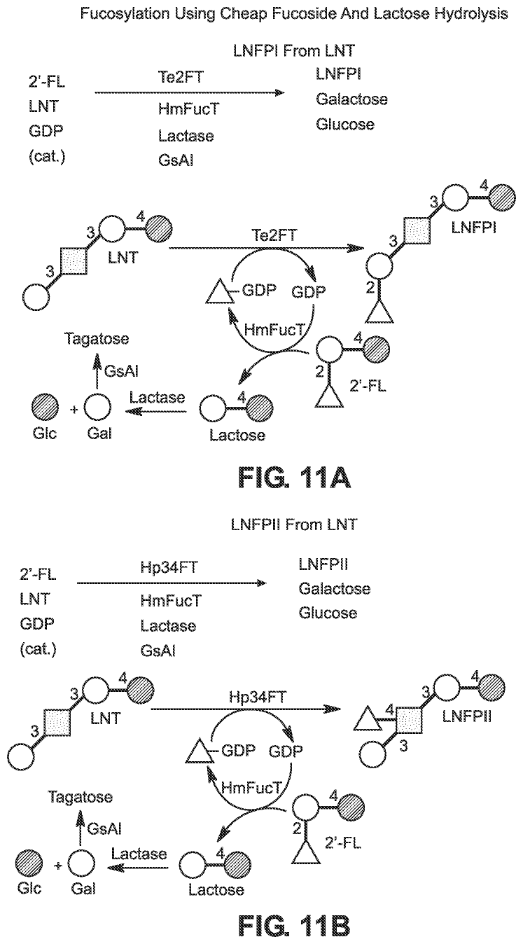

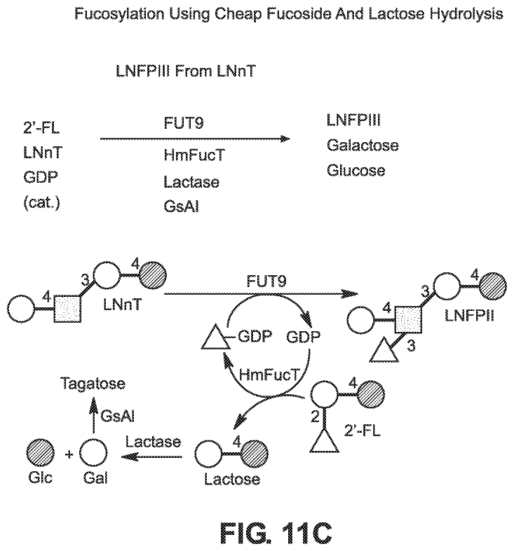

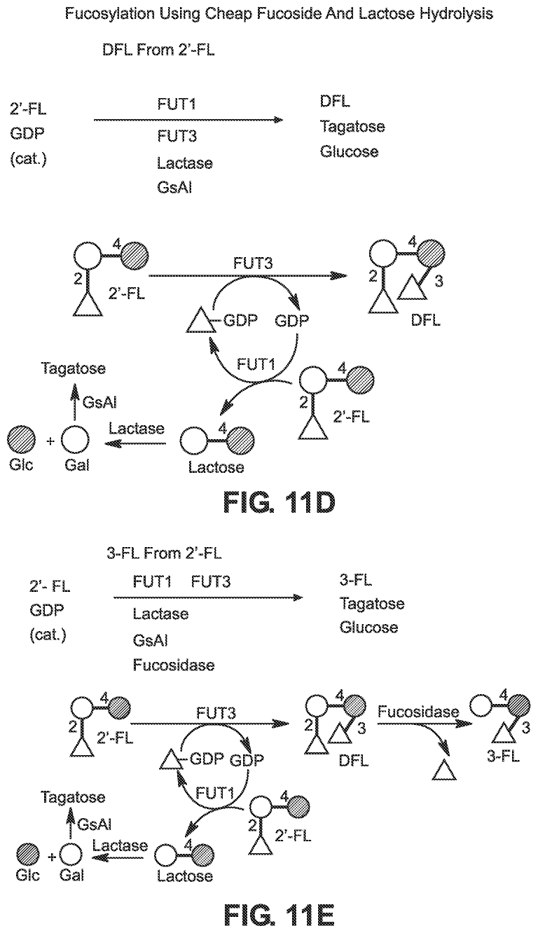

FIGS. 11A-11E depict fucosylation using fucoside and lactose hydrolysis. GDP-1-fucose is the nucleotide-sugar used in each of FIG. 11A-11C.

FIG. 11A depicts a route for obtaining LNFPI from LNT. GDP-Fuc in the presence of 2′-FL, LNT, Te2FT, HmFucT, lactase, and GsAI provides LNFPI.

FIG. 11B depicts a route for obtaining LNFPII from LNT. GDP-Fuc in the presence of 2′-FL, LNT Hp34FT, HmFucT, lactase, and GsAI provides LNFPII.

FIG. 11C depicts a route for obtaining LNFPIII from LNnT. GDP-Fuc in the presence of 2′FL, LNnT FUT9, HmFucT, lactase, and GsAI provides LNFPIII.

FIG. 11D depicts a route for obtaining DFL from 2′-FL. GDP-Fuc in the presence of 2′-FL, FUT1, FUT3, lactase, GsA1, and fucosidase provides 3-FL, tagatose, and glucose.

FIG. 11E depicts a route for obtaining 3-FL from 2′-FL. GDP-Fuc in the presence of 2′-FL, FUT1, FUT3, lactase, and GsAI. Lactase depletes lactose in this route. Instead of lactase, any other enzymatic or nonenzymatic means may be used to deplete lactose. Additionally, GsAI may be added to convert galactose to tagatose to augment the reaction conversion. See Example 5a.D and Example 5a.E.

In some embodiments, a fucosyltransferase catalyzes the transfer of GDP-fucose (GDP-Fuc) onto either Galactose, Glucose, GlcNAc or GalNAc. The methods depicted in FIG. 3A-C and FIG. 11A-E employ fucosyltransferases to transfer a fucosyl group from a sugar nucleotide donor to an acceptor.

In certain embodiments, the fucosyltransferase is an α-1,2-fucosyltransferase (EC 2.4.1.69). In particular embodiments, the α-1,2-fucosyltransferase is Te2FT from Thermosynechococcus elongatus, WbgL from Escherichia coli, HmFucT from Helicobacter mustelae, FUT1 from Homo sapiens, or FUT2 from Homo sapiens.

In certain embodiments, the fucosyltransferase is an α-1,3-fucosyltransferase (EC 2.4.1.152, EC 2.4.1.214). In particular embodiments, the α-1,3-fucosyltransferase is HpFucT from Helicobacter pylori, Bf1,3FT from Bacteroides fragilis, Hp3/4FT from Helicobacter pylori, FUT3 from Homo sapiens, FUT4 from Homo sapiens, FUT5 from Homo sapiens, FUT6 from Homo sapiens, FUT7 from Homo sapiens, FUT9 from Homo sapiens, or FUT11 from Homo sapiens.

In certain embodiments, the fucosyltransferase is an α-1,4-fucosyltransferase (EC 2.4.1.65). In particular embodiments, the α-1,4-fucosyltransferase is Hp3/4FT from Helicobacter pylori or FUT2 from Homo sapiens.

In certain embodiments, the fucosyltransferase is an α-1,6-fucosyltransferase (EC 2.4.1.68). In a particular embodiment, α-1,6-fucosyltransferase is FUT8 from Homo sapiens.

In certain embodiments, the fucosyltransferase is selected from the fucosyltransferases depicted in the Figures.

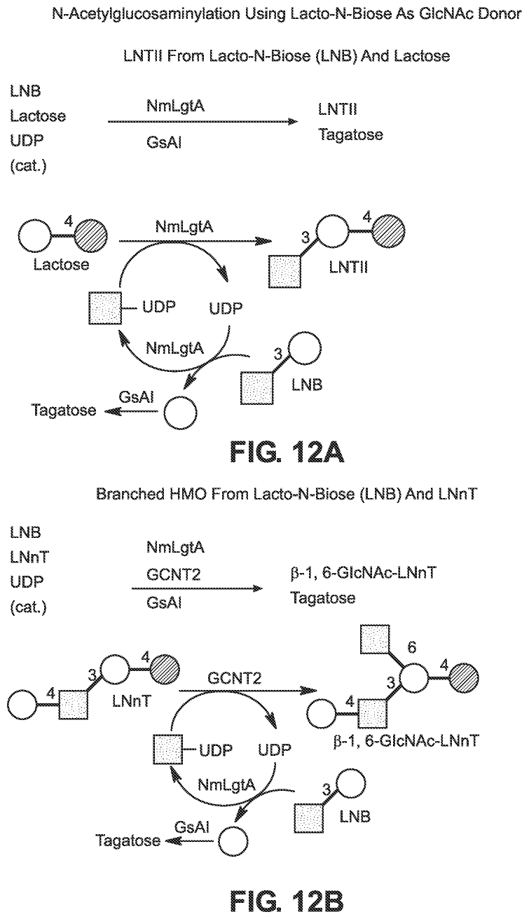

FIG. 12A and FIG. 12B depict routes N-acetylglucosaminylation using lacto-N-biose (LNB) as a GlcNAc donor.

FIG. 12A depicts a route for obtaining LNTII and tagatose from LNB and lactose. UDP-N-Acetylglucosamine (UDP-GlcNAc) is prepared from UDP in the presence of NmLgtA and GsAI.

FIG. 12B depicts a route for obtaining branched human milk oligosaccharides from LNB and LNnT in the presence of UDP, NmLgtA, GCNT2, and GsAI. β-2,6-GlcNAc-LNnT and tagatose is obtained. In the route depicted GsAI is present to deplete galactose. Any other enzymatic or nonenzymatic means to deplete galactose may be used instead of GsAI.

In some embodiments, an N-acetylglucosaminyltransferase catalyzes the transfer of UDP-N-acetylglucosamine (UDP-GlcNAc) to Galactose, Mannose or GlcNAc.

In certain embodiments, the N-acetylglucosaminyltransferase is β-1,3-N-acetylglucosaminyltransferase (EC 2.4.1.79, EC 2.4.1.149, EC 2.4.1.222). In particular embodiments, the β-1,3-N-acetylglucosaminyltransferase is HpLgtA form Helicobacter pylori, NmLgtA from Neisseria meningitidis, HP1105 from Helicobacter pylori, B3GNT2 from Homo sapiens, B3GNT3 from Homo sapiens, B3GNT4 from Homo sapiens, B3GNT7 from Homo sapiens, B3GNT8 from Homo sapiens, or B3GNT9 from Homo sapiens.

In certain embodiments, the N-acetylglucosaminyltransferase α-1,4-N-acetylglucosaminyltransferase (EC 2.4.1.223, EC 2.4.1.224).

In certain embodiments, the N-acetylglucosaminyltransferase is a β-1,2-N-acetylglucosaminyltransferase (EC 2.4.1.101, EC 2.4.1.143). In particular embodiments, the β-1,2-N-acetylglucosaminyltransferase is MGAT1(GlcNAcT-I) from Homo sapiens or MGAT2 (GlcNAcT-II) from Homo sapiens.

In certain embodiments, the N-acetylglucosaminyltransferase is a β-1,4-N-acetylglucosaminyltransferase (EC 2.4.1.144, EC 2.4.1.212). In particular embodiments, the β-1,4-N-acetylglucosaminyltransferase is MGAT3 (GlcNAcT-III) from Homo sapiens, MGAT4A (GlcNAcT-IV) from Homo sapiens, MGAT4B (GlcNAcT-IV) from Homo sapiens, or MGAT4C (GlcNAcT-IV) from Homo sapiens.

In certain embodiments, the N-acetylglucosaminyltransferase is a β-1,6-N-acetylglucosaminyltransferase (EC 2.4.1.102, EC 2.4.1.150, EC 2.4.1.155). In particular embodiments, the β-1,6-N-acetylglucosaminyltransferase is GCNT2A from Homo sapiens, GCNT2B from Homo sapiens, GCNT2C from Homo sapiens, GCNT3 from Homo sapiens, GCNT4 from Homo sapiens, or MGAT5 (G1cNACT-V) from Homo sapiens.

In certain embodiments, the N-acetylglucosaminyltransferase is selected from the N-acetylglucosaminyltransferases depicted in the Figures.

In certain embodiments of this invention, the starting systems of reactants and enzymes comprise, consist essentially of, or consist of those described in FIGS. 1-12.

The invention uses sugars as an energy source. An activated sugar (sugar-nucleotide or sugar-nucleotide compound) is therefore a starting material and an energy source. The energy source is hydrolysis of more complex sugars into more simple sugars. This aids in driving reactions of this invention to completion. In one embodiment, sucrose is employed for galactosylation, and hydrolysis of lactose is used for both sialylation and fucosylation. In another embodiment, lacto-N-biose is used as a GlcNAc donor in N-acetylglucosaminylation reactions. In another embodiment, hydrolysis of 3′-SL is used in sialylation reactions. In another embodiment, 2′-FL is used in fucosylation reactions.

The energy driver is a larger saccharide being converted to smaller saccharide. For example, energy is derived from a disaccharide breaking down, hydrolyzing, into a monosaccharide, such as sucrose breaking down into fructose and glucose. That is sucrose and water being converted into fructose and glucose via hydrolysis.

The activated sugar is regenerated in the reaction mixture. This allows the nucleotide to be present in catalytic amounts. As depicted in each Figure, transfer of a sugar to an acceptor then results in formation of a free nucleotide. The nucleotide then reacts with a sugar to form an activated sugar. In FIG. 1, the nucleotide is added to glucose to form UDP-glucose, in FIG. 2 the nucleotide is added to N-acetyl neuraminic acid to form CMP-Neu5Ac, and in FIG. 3 the nucleotide is added to 2′-FL to form GDP-2′-FL.

Without being bound by theory, manganese (Mn) and high sucrose concentrations lead to enzyme deactivation. In certain embodiments, a process of this invention employs magnesium (Mg) instead of manganese (Mn). For example, Mn catalyzes the decomposition of UDP to UMP, and UMP leads to the reductive inactivation of a co-factor NAD+ in GalE. In certain embodiments, a process of this invention employs magnesium (Mg) instead of manganese (Mn) and relatively low concentrations of sucrose, for example, 50-100 mM.

Manganese (Mn) and inorganic phosphate form insoluble byproducts, particularly at higher industrially relevant concentrations, that make it unsuitable for packed bed reactor applications and that result in the removal of the critical metal cofactor (Mn) from the solution phase which reduces the activity of many glycosyltransferases. Embodiments of this invention employ Mg and therefore avoid these disadvantages.

Sugar nucleotides that have affinity for the enzymes of the invention may be employed in the processes disclosed herein. Enzymes that may be employed in this invention include those bioengineered for affinity. In certain embodiments, the activated sugar comprises, consists essentially of, or consists of: Galactose-UDP, Galactose-ADP, Sialic Acid-CMP, and Fucose-GDP. In certain embodiments, the activated sugar comprises, consists essentially of, or consists of: Galactose-UDP, Sialic Acid-CMP, and Fucose-GDP.

In one embodiment, this invention provides a biocatalytic process for preparing a saccharide-acceptor comprising the steps of:
a. contacting a saccharide and a nucleotide to form a saccharide-nucleotide, wherein the nucleotide is present in a catalytic amount; and
b. contacting the saccharide-nucleotide and an acceptor in the presence of a transferase to form the saccharide-acceptor.

In certain embodiments, the saccharide is a monosaccharide. In certain embodiments, the monosaccharide is galactose, sialic acid, or 1-fucose.

In another embodiment, this invention provides a process for preparing a saccharide compound, comprising the steps of:
a. contacting a monosaccharide selected from galactose, sialic acid, and 1-fucose and a catalytic amount of a nucleotide selected from UDP, ADP, CMP, and GDP to form a saccharide-nucleotide compound; and
b. contacting the saccharide-nucleotide compound and an acceptor compound in the presence of a transferase to obtain the saccharide.

In another embodiment, this invention provides a process for preparing a saccharide compound, comprising the steps of:
a. contacting a galactose saccharide unit and a catalytic amount of a nucleotide selected from UDP and ADP or a sialic acid or 1-fucose saccharide unit and a catalytic amount of a nucleotide selected from CMP, and GDP to form a saccharide-nucleotide compound.
b. contacting the saccharide-nucleotide compound and an acceptor compound in the presence of a transferase to obtain the saccharide.

In another embodiment, this invention provides for galactosylation of an acceptor compound, comprising the steps of:
a. contacting galactose and a nucleotide to form a galactose-nucleotide compound, wherein the nucleotide is present in a catalytic amount; and
b. contacting the galactose-nucleotide compound and an acceptor compound in the presence of a transferase to galactosylate the acceptor compound.

In certain embodiments, the galactose is obtained from sucrose in situ.

In other embodiments, the process is driven to high conversion via the conversion of Sucrose to Glc-UDP and fructose.

In another embodiment, this invention provides for a process for sialylation of an acceptor compound, comprising the steps of:
a. contacting sialic acid and a nucleotide to form a sialyl-nucleotide compound, wherein the nucleotide is present in a catalytic amount; and
b. contacting the sialyl-nucleotide compound and a compound with an acceptor compound in the presence of a transferase to sialylate the acceptor compound.

In certain embodiments, the sialic acid is obtained from 3′-SL or 6′-SL in situ.

In other embodiments, the process is driven to high conversion via the conversion of lactose to galactose and glucose.

In another embodiment, this invention provides for a process for fucosylation of an acceptor compound, comprising the steps of:
a. contacting a fucose and a nucleotide to form a fucose-nucleotide compound, wherein the nucleotide is present in a catalytic amount; and
b. contacting the fucose-nucleotide compound and an acceptor compound in the presence of a transferase to fucosylate the acceptor compound.

In certain embodiments the fucose is 1-fucose and is obtained from 2′-FL in situ. In other embodiments the process is driven to high conversion via the conversion of sucrose to Glc-UDP and fructose. In yet other embodiments of this invention, the acceptor compound is a monosaccharide, a disaccharide, an oligosaccharide, or a polysaccharide. In yet other embodiments of this invention there is substantially no ATP added to the process or generated by the process.

In another embodiment, this invention provides a process for producing LNT and fructose, comprising the step of contacting LNTII, sucrose, a transferase, a sucrose synthase, an epimerase, and UDP to produce LNT and fructose, wherein galactose-UDP is produced, and wherein the UDP is present in about 0.01 molar percent to about 10 molar percent of the galactose or the sucrose. In certain embodiments, the transferase is Cvb3GalT. In other embodiments, the transferase transferase is B3GALT5. In certain embodiments the epimerase is GalE.

In another embodiment, this invention provides a process for producing LNnT and fructose, comprising contacting LNTII, sucrose, a glycosyltransferase, a sucrose synthase, an epimerase, and UDP to produce LNnT and fructose, wherein the UDP is present in about 0.01 molar percent to about 10 molar percent of the galactose or sucrose. In certain embodiments the glycosyltransferase is a LgtB. In other embodiments the LgtB is NmLgtB. In certain embodiments, the glycosyltransferase is B4GALT1. In certain embodiments, the glycosyltransferase is B4GALT1. In certain embodiments, the epimerase is GalE.

In another embodiment, this invention provides a process for preparing LNT and fructose, comprising the steps of contacting GlcNAc, 2-chloro-1,3-dimethylimidazolinium chloride, and a base to obtain a reaction mixture comprising GlcNAc-Oxa; and contacting the reaction mixture comprising GlcNAc-Oxa, lactose, sucrose, an aminidase, a transferase, a sucrose synthase, and an epimerase, to provide LNT and fructose, wherein the UDP is present in about 0.01 molar percent to about 10% molar percent of the lactose or the sucrose.

In certain embodiments, the aminidase is Bbh1. In certain embodiments, the transferase is Cvb3GalT. In certain embodiments, the epimerase is GalE. In certain embodiments, the base is triethylamine.

In another embodiment, this invention provides a process for preparing lactose and fructose, comprising contacting glucose, sucrose, a lactose synthase, a sucrose synthase, an epimerase, and UDP, to provide lactose and fructose, wherein the UDP is present in about 0.01 molar percent to about 10 molar percent of the glucose and or the sucrose. In certain embodiments, the epimerase is GalE.

In another embodiment, this invention provides a process for preparing LSTa, galactose, and glucose, comprising contacting 3′-SL, LNT, CMP, a sialyltransferase, and a lactase to provide LSTa, galactose, and glucose, wherein the CMP is present in about 0.01 molar percent to about 10 molar percent of the 3′-SL. In certain embodiments the sialyltransferase is ST3GAL3.

In another embodiment, this invention provides a process for preparing LSTb comprising contacting LNT, 3′-SL, and catalytic amounts of CMP in the presence of ST3GAL4, ST3GAL3, ST6GALNAC5, Lactase and α2-3 Neuraminidase S, wherein the CMP is present in about 0.01 molar percent to about 10 molar percent of the 3′-SL, wherein lactose is obtained and then processed to glucose and galactose by lactase, and wherein α2-3 Neuraminidase S converts DSLNT to LSTb.

In another embodiment, this invention provides a process for preparing LSTc, galactose, and glucose, comprising contacting 3′-SL, LNnT, CMP, a sialyltransferase, and a lactase to provide LSTc, galactose, and glucose, wherein the CMP is present in about 0.01 molar percent to about 10 molar percent of the 3′-SL. In certain embodiments, the sialyltransferase is ST3GAL3.

In another embodiment, this invention provides a process for preparing LSTd, galactose, and glucose, comprising contacting 3′-SL, LNnT, CMP, a sialyltransferase, and a lactase to provide LSTd, galactose, and glucose, wherein the CMP is present in about 0.01 molar percent to about 10 molar percent of the 3′-SL. In certain embodiments, the sialyltransferase is ST3GAL3.

In another embodiment, this invention provides a process for preparing DSLNT, galactose, and glucose, comprising contacting 3′-SL, LNT, CMP, a sialyltransferase, and a lactase to provide DSLNT, galactose, and glucose, wherein the CMP is present in about 0.01 molar percent to about 10% molar percent of the 3′-SL. In certain embodiments, the sialyltransferase is ST3GAL3 and ST6GALNAC5.

In another embodiment, this invention provides a process for preparing LNFPI, galactose, and glucose, comprising contacting 2′-FL, LNT, GDP, Te2FT, HmFucT, and lactase to provide LNFPI, galactose, and glucose wherein the GDP is present in about 0.01 molar percent to about 10 molar percent of the 2′-FL In certain embodiments, the transferase is Te2FT and HmFucT.

In another embodiment, this invention provides a process for preparing LNFPII, galactose, and glucose, comprising contacting 2′-FL, LNT, GDP, Hp34FT, HmFucT, and lactase, wherein the GDP is present in about 0.01 molar percent to about 10 molar percent of the 2′-FL. In certain embodiments, the transferase is Hp34FT and HmFucT.

In another embodiment, this invention provides a process for preparing LNFPIII, galactose, and glucose, comprising contacting 2′-FL, LNnT, GDP, FUT9, HmFucT, and lactase, to provide LNFPIII, wherein the GDP is present in about 0.01 molar percent to about 10 molar percent of the 2′-FL. In certain embodiments, the transferase is FUT and HmFucT.

In another embodiment, this invention provides a process for preparing 3-FL, comprising contacting 2′-FL, GDP, FUT1, FUT3, lactase, GsAI, and fucosidase to provide 3-FL, tagatose, and glucose, wherein the GDP is present in about 0.01 molar percent to about 10 molar percent of the 2′-FL.

In another embodiment, this invention provides a process for preparing LNTII, comprising contacting LNB, lactose, UDP, NmLgtA, and GsAI to provide 3-FL, tagatose, and glucose, wherein the UDP is present in about 0.01 molar percent to about 10 molar percent of the LNB.

In another embodiment, this invention provides a process for preparing LNTII, comprising contacting LNB, lactose, UDP, NmLgtA, and GsAI to provide 3-FL, tagatose, and glucose, wherein the UDP is present in about 0.01 molar percent to about 10 molar percent of the LNB.

In another embodiment, this invention provides a process for preparing β-1,6-GlcNAc-LNnT, comprising contacting LNB, LNnT, UDP, NmLgtA, GCNT2, and GsAI to provide β-1,6-GlcNAc-LNnT and tagatose, wherein the UDP is present in about 0.01 molar percent to about 10 molar percent of the LNB.

In certain embodiments, the sialic acid is N-acetyl neuraminic acid (Neu5Ac).

In certain embodiments, the sugars are in their naturally occurring isomeric forms. In some embodiments, the glucose is D-glucose. In some embodiments, galactose is D-galactose. In some embodiments, fucose is L-fucose. In some embodiments, tagatose is D-tagatose. In some embodiments, N-acetylglucosamine (GlcNAc) is N-Acetyl-D-glucosamine. In some embodiments, fructose is D-fructose.

In other embodiments, the nucleotide is present in the catalytic amount of about 0.01 to about 10 molar percent relative to the saccharide amount. The relevant saccharide is either the staring saccharide or the saccharide that is generated and reacts with the nucleotide.

In some embodiments, the nucleotide monophosphates and diphosphates used in this invention are used in catalytic amounts relative to the molarity of sugar that is bound to the monophosphate or diphosphate. In another embodiment, the nucleotide monophosphates and diphosphates used in this invention are used in catalytic amounts relative to the molarity of starting material sugar or other acceptor. These nucleotides include natural, artificial, or synthetic nucleotides. As used herein, a “catalytic amount” of nucleotide ranges are in an amount of precisely, about, up to, or less than, for example 0.01% to 10% moles of nucleotide to moles of sugar, either sugar donor or acceptor molecule. As used herein, a “catalytic amount” of nucleotide ranges are in an amount of precisely, about, up to, or less than, for example 0.001% to 10% moles of nucleotide to moles of either sugar donor or acceptor molecule. In other embodiments, the nucleotide monophosphates or nucleotide diphosphates are in an amount of precisely, about, up to, or less than, for example 0.001% to 1% (mol percent). In certain embodiments, the nucleotide monophosphates and diphosphates are in an amount of precisely, about, up to, or less than, for example 0.01% to 10% (mol percent). In other embodiments, the nucleotide monophosphates or nucleotide diphosphates are in an amount of precisely, about, up to, or less than, for example 0.1% to 1% (mol percent). In other embodiments, precisely, about, up to, or less than, for example, the nucleotide monophosphates and nucleotide diphosphates are added in an amount of 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, or 0.09%, (mol percent). In other embodiments, the nucleotide monophosphates and nucleotide diphosphates are added in an amount of precisely, about, up to, or less than, for example, 0.1%, 0.2%, 0.3%, 0.4%, 0.5%, 0.6%, 0.7%, 0.8%, 0.9%, or 1.0% (mol percent). In other embodiments, the nucleotide monophosphates and nucleotide diphosphates are added in amounts of precisely, about, up to, or less than, for example, the monophosphates and diphosphates are added in an amount of 0.01%, 0.1%, 0.3%, 1.0%, or 10% (mol percent). In other embodiments, the nucleotide monophosphates and nucleotide diphosphates are added in amounts of precisely, about, up to, or less than, for example, the monophosphates and diphosphates are added in amount of 0.1, 0.3, or 1.0% (mol percent). In other embodiments, the nucleotide monophosphates and nucleotide diphosphates are added in amounts of precisely or about 0.001%, 0.01%, 0.1%, 1.0%, or 10%. In other embodiments, the monophosphates and diphosphates are added in amounts of precisely or about 0.1%, 1.0%, or 10%. In other embodiments, the monophosphates and diphosphates are added in amounts of precisely or about 0.1%, 0.3%, or 1.0% (mol percent). FIG. 1-14 depict embodiments employing catalytic nucleotides.

In some embodiments, plant sugars are used to create glycosylated compounds including, but not limited to, galactosylated compounds, sialylated compounds, and fucosylated compounds, wherein the galactose, sialic acid, and fucose is added to a hydroxy group including, but not limited to, a hydroxy group of a sugar. In another embodiment lacto-N-biose is used as a GlcNAc donor to be added to a hydroxy group of a sugar in an N-acetylglucosaminylation reaction. Accordingly, plant sugars or simple HMOs produced by microbial fermentation, or from animal origin, are used to create more complex saccharides including, but not limited to, oligosaccharides found in animals. In certain embodiments, the compounds include, but are not limited to LNT, LNnT, lactose, GOS, LSTa, LSTc, LSTd, DSLNT, LNFPI, LNFPII, LNFPIII. In other embodiments, the compounds include, but are not limited to, LSTb and 3-FL. In other embodiments, plant sugars are used to synthesize lactose derivatives by chemoenzymatic processes to obtain lactose derivatives including, but not limited to, lactobionic acid, lactitol, lactosucrose, galacto-oligosaccharides (GOS), lactulose, and HMOs (see FIG. 5). M. G. Ganzle et al., International Dairy Journal, 18 (2008) 685-694.

As depicted in FIG. 5, lactobionic acid is obtained from lactose via an oxidation reaction. Lactitol is obtained from lactose via a reduction reaction. Lactosucrose is obtained from lactose via fructosyl transfer. Galacto-oligosaccharides (β-(1,4) or (β-(1,6) linkages) are obtained from lactose via transgalactosylation. Lactulose is obtained from lactose via isomerization.

In certain embodiments, the products obtained from the processes of this invention are not animal derived. Such products are advantageous in markets where animal-free products are desired.

This approach obviates the need for ATP and cofactor recycling. In certain embodiments, the activated sugars are activated galactose, activated Neu5Ac, activated fucose, or activated glucose. In another embodiment, the activated sugar is N-acetylglucosamine (GlcNAc). These activated sugars are generated in situ. The reactions are designed to be nucleotide triphosphate free and to avoid reagent and co-factor recycling or regeneration. This reduces the number of enzymes needed to produce each oligosaccharide and avoids acidification. This reduces costs and leads to higher yields and higher purity products.

In certain embodiments, more than one saccharide secondary product is produced in the reaction mixture. In certain embodiments, glucose and galactose are both produced as secondary products in a single reaction mixture.

In certain embodiments, a processing enzyme is employed to convert a secondary product into a secondary product derivative. In certain embodiments, the conversion is beneficial in driving the reaction to completion towards formation of the principal product. In other embodiment, the conversion can be tailored to obtain a more desired secondary product derivative for recovery and reuse.

In certain embodiments, a processing enzyme is employed to convert a principal product to a principal product derivative. In certain embodiments, the conversion is beneficial to obtain a more desired principal product derivative for recovery and reuse.

In certain embodiments, more than one processing step may be employed to further convert either a principal product derivative or a secondary product derivative. The number of processing steps employed depends on driving the reaction to completion and obtaining a desired final product. Certain galactosylation methods of this invention employs none or one processing of either the principal product or the secondary product. In certain embodiments, no processing step is employed in galactosylation methods of this invention. Certain sialylation, fucosylation, and N-acetylglucosaminylation methods employ none, one, or two processing of either the principal product or the secondary product. In certain sialylation, fucosylation, and N-acetylglucosaminylation embodiments, one processing step converts the principal product to a principal product derivative or the secondary product to a secondary product derivative.

Some embodiments of this invention employ a processing enzyme that either hydrolyzes, oxidizes, or isomerizes a carbohydrate or oligosaccharide.

In certain embodiments, the processing enzyme is an enzyme that hydrolyzes lactose. In particular embodiments, the enzyme the hydrolyzes lactose is Lactase (β-galactosidase) (EC 3.2.1.23). In more particular embodiments, the Lactase is (β-galactosidase from Aspergillus niger, β-galactosidase from Escherichia coli, (β-galactosidase from Aspergillus oryzae, or β-galactosidase from Kluyveromyces lactis.

In certain embodiments, the processing enzyme is an enzyme that oxidizes lactose carbohydrates. In particular embodiments, the enzyme that oxidizes lactose carbohydrate is a glucose oxidase (EC 1.1.3.4). In more particular embodiments, the glucose oxidase is Glucose oxidase from Aspergillus niger, Glucose oxidase from Penicillium amagasakiense, Glucose oxidase from Penicillium notatum (chrysogenum), Glucose oxidase from Penicillium variabile, Galactose oxidase (EC 1.1.3.9), Galactose oxidase from Dactylium dendroides, Galactose oxidase from Polyporus circinatus, Galactose oxidase from Fusarium graminearum, Galactose oxidase from Drosophila melanogaster.

In particular embodiments, the enzyme that oxidizes lactose carbohydrate is an enzyme that oxidizes lactose to Lactobionic acid. In more particular embodiments, the enzyme that oxidizes lactose carbohydrate is Carbohydrate oxidase from Microdochium nivale, Lactose oxidase from Myrmecridium flexuosum, Lactose oxidase from Sarocaldium oryzae, Lactose oxidase from Paraconiothyrium sp., Galactose oxidase (EC 1.1.3.9) from Polyporus circinatus, Cellobiose dehydrogenase (EC 1.1.99.18) from Sclerotium rolfsii, Cellobiose quinone oxidoreductase (EC 1.1.5.1) from Phanerochaete chrysosporium, Cellobiose quinone oxidoreductase (EC 1.1.5.1) from Spotoyrichum pulverolentum, carbohydrate oxidoreductase from Sarocladiumoryzae, carbohydrate oxidoreductase from Paraconiothyrium sp., or Glucooligosaccharide oxidase GOOX from Acremonium strictum.

In certain embodiments, the processing enzyme is an enzyme that isomerizes carbohydrates. In particular embodiments, the enzyme that isomerizes carbohydrates is a glucose isomerase (xylose isomerase) (EC 5.3.1.5). In more particular embodiments, the glucose isomerase is Glucose isomerase from Streptomyces murinus, Glucose isomerase from Actinoplanes missouriensis, Glucose isomerase from Bacillus coagulans, Glucose isomerase from Arthrobacter, or Glucose isomerase from Xanthomonas campestris.

In particular embodiments, the enzyme that isomerizes carbohydrates is L-Arabinose isomerase (Galactose isomerase) (EC 5.3.1.4). In more particular embodiments, the L-Arabinose isomerase is L-Arabinose isomerase from Geobacillus stearothermophilus L-Arabinose isomerase from Lactobacillus sakei, L-Arabinose isomerase from Bacillus subtilis, L-Arabinose isomerase from Escherichia coli or L-Arabinose isomerase from Thermoanaerobacter mathranii.

In some embodiments, the processing enzyme is an enzyme that hydrolyze carbohydrates. In certain embodiments, enzyme that hydrolyze carbohydrate is a fucosidase. Fucosidases are enzymes that catalyze the hydrolysis fucose from a glycan, carbohydrate, or glycan bearing moiety. In particular embodiments, the fucosidase is α-1,2-fucosidase (EC 3.2.1.63), α-1,3-fucosidase (EC 3.2.1.111) or α-1,4-fucosidase (EC not known).

In certain embodiments, enzyme that hydrolyze carbohydrate is a sialidase (Neuraminidase). Sialidases are enzymes that catalyze the hydrolysis of sialic acid from a glycan, carbohydrate, or glycan bearing moiety. In particular embodiments, the sialidase is an xo-α-sialidase. In more particular embodiments, the exo-a-sialidase is α2-3 Neuraminidase S, α2-3,6,8,9 Neuraminidase A, α2-3,6,8 Neuraminidase.

In particular embodiments, the sialidase is an Endo-a-sialidase. In a more particular embodiment, the sialidase is α2,8 Neuraminidase.

In certain embodiments, a processing enzyme is selected from the fucosyltransferases depicted in the Figures.

In certain embodiments of the galactosylation reactions exemplified herein fructose is the secondary product produced and may be recovered or processed for further use. In certain embodiments of the sialylation and fucosylation reactions exemplified herein galactose and glucose are the secondary products produced and may be recovered or processed for further use. In certain embodiments of the acetylglucosaminylation reactions exemplified herein lactose or galactose are the secondary products produced and may be recovered or processed (e.g., to tagatose) for further use.

The nucleotides may also be recovered. In the embodiments exemplified herein, UDP or ADP may be recovered in galactosylation reactions, CMP may be be recovered in the sialylation reactions, and GDP may be recovered in the fucosylation reactions.

In certain embodiments, glycans are obtained in the methods of this invention.

Glycans, are carbohydrate-based compounds featuring one or more monosaccharides linked with a glycosidic bond, including N-linked and O-linked bonds. Activated monosaccharides, oligosaccharides, polysaccharides, plant glycans, animal glycans, and microbe glycans are all within the scope of this invention as are glycoconjugates, such as glycolipid, glycopeptides, glycoproteins, and proteoglycans. Glycans also include humanized glycoproteins, humanized antibodies, and glycoconjugate vaccines. Riley, et al. “Glycosylation in health and disease” Nature Reviews Nephrology volume 15, pages 346-366 (2019). Rappuoli, “Glycoconjugate vaccines: Principles and mechanisms” Science Translational Medicine 29 Aug. 2018: Vol. 10, Issue 456. The forgoing are incorporated by reference in their entirety.

Compounds that are acceptors may be derivatized with a glycosyl group according to this invention. Any organic compounds comprising at least one alcohol (hydroxyl) functional group may be an acceptor and therefore glycosylated by the processes of this invention. Such compounds may include, but are not limited to, rare sugars, activated sugars, HMOs, glycans with sugar modifications, glycosylated small molecules, polymerized fiber sugars, inulins, levans, gluconic acid, invert sugar, flavors, and fragrances. Organic compounds bearing at least one alcohol functional group (organic hydroxy functional group) that may be glycosylated according to this invention include, but are not limited to, Steviol, a class of compounds called Rebaudiosides, phenolic compounds, proteins with O-glycosylation and N-glycosylation sites: serine, threonine asparagine as well as compounds related thereto.

### Integrated Fucosylation Pathway

In another aspect the present invention provides an integrated pathway using immobilized enzymes to perform a synthesis of oligosaccharides, including embodiments utilizing a one-pot synthesis of fucosolyated oligosaccharides starting from fructose or glucose. This lowers the cost of production of fucosylated HMOs vs using fucose as a starting material. A glycan to be fucosylated may be prepared separately for use a one-pot synthesis of this invention.

The invention provides cell-free de-novo synthesis of fucosylated glycan starting from simple sugars. An integrated pathway of immobilized enzymes perform the one-pot fucosylation of oligosaccharides or antibody-glycan conjugates starting from fructose or glucose, rather than fucose. The system of immobilized enzymes comprises, consists essentially of, or consists of:
An enzyme that converts fructose and/or glucose to mannose
A set of enzymes to convert mannose to GDP-mannose
A set of enzymes that converts GDP-mannose to GDP-fucose
An enzyme that transfers fucose to an oligosaccharide backbone from GDP-fucose
A set of enzymes that recycle cofactors from stochiometric reagents

The HMO 2′-FL may be produced from lactose. The last transferase of the enzyme cascade can be changed to fucosylate other oligosaccharides backbones. The switch to a different fucosyltransferase constitutes a separate HMO production, again via a one-pot system. For example, one fucosyl transferase would be employed to make 2′-FL, another fucosyl transferase would be employed LNFP1, and another fucosyl transferase would be employed SM to makes LNFPII etc. Thus, modularity arises from changing the last enzyme of the system (i.e., the fucosyl transferase).

The methods provide high volumetric productivity, are cost effective, and are non-GMO label.

The immobilized enzymes may be coupled to a processing plant producing lactose, fructose, or glucose feeds for continuous flow manufacturing. The invention may be integrated to a biorefinery to convert glucose or fructose to fucosylated HMOs.

The last transferase of the enzyme cascade can be changed to transfer GDP-fucose to other oligosaccharides backbones for fucosylation reactions.

The cascade of enzymes can produce GDP-mannose that can also be polymerized to mannans via transferase enzymes.

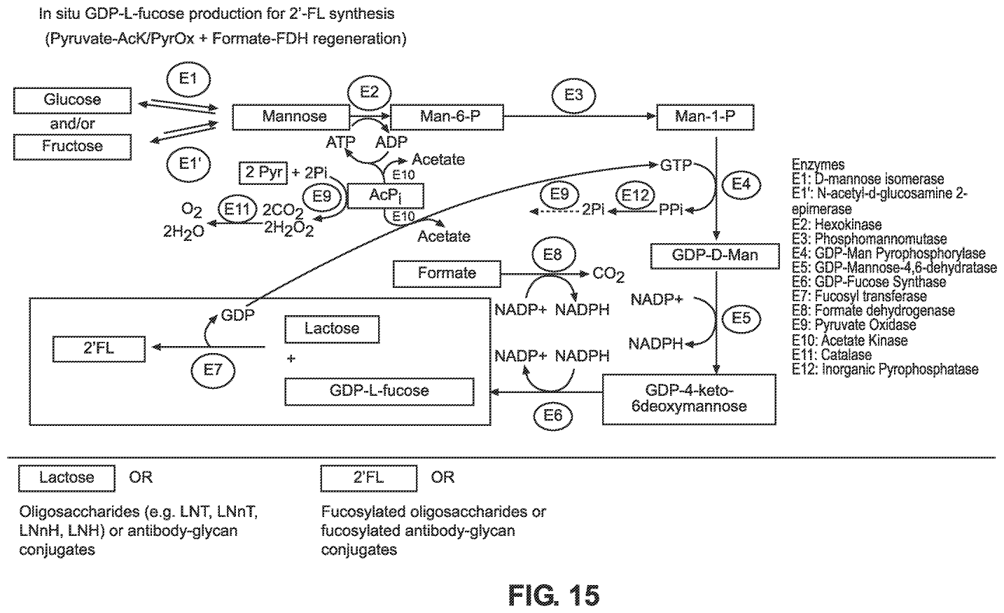

As depicted in FIG. 15, glucose and/or fructose is converted to GDP-L-fucose in an integrated synthesis. Enzyme E1 converts glucose to mannose. Alternatively, enzyme E1′ converts fructose to mannose. Then enzyme E2 converts mannose to mannose-6-phosphate. Enzyme E3 converts mannose-6-phosphate to mannose-1-phosphate. Enzyme E4 converts mannose-1-phosphate to GDP-D-mannose. Enzyme E5 converts GDP-D-mannose to GDP-4-keto-6-deoxymannose. Enzyme E6 converts GDP-4-keto-6-deoxymannose to GDP-L-fucose. 2′FL is obtained from GDP-L-fucose, lactose, and enzyme E7. All enzymes are immobilized

Regeneration enzymes are also depicted in FIG. 15. Enzymes E8, E9, E10, and E11, and E12 regenerate reagents to allow for the integrated synthesis. The following reaction occur: GDP to GTP (E10), inorganic diphosphate to inorganic phosphate (E12), carbon dioxide and hydrogen peroxide to oxygen and water (E11), ADP to ATP (E10), acetate phosphate to acetate (E10), inorganic phosphate and pyruvate to acetate phosphate (E9), and formate to carbon dioxide (E8).

In one embodiment, enzymes used are Glk, RfbK, RfbM, Gmd, and GFS along with regeneration enzymes Pyruvate-AcK, PyrOx, and FDH. The enzymes convert glucose and/or fructose to mannose, then mannose to mannose-6-phosphate, then mannose-6-phosphate to mannose-1-phosphate, then mannose-1-phosphate to GDP-D-mannose, then GDP-D-mannose to GDP-4-keto-6-deoxymannose, and then GDP-4-keto-6-deoxymannose to GDP-L-fucose. Further reactions involve fucosylation of any sugar residue including, but not limited to, oligosaccharides or fucosylated antibody-glycan conjugates. In the presence a fucosyl transferase and GDP-L-fucose, lactose is converted to 2′FL via fucosyl transferase. Similarly, an oligosaccharide is converted to a fucosylated oligosaccharide or an antibody-glycan conjugate is converted to a fucosylated antibody-glycan conjugate. The depicted fucosyl transferase of the enzyme cascade may be changed to transfer GDP-fucose to other oligosaccharides backbones for fucosylation reactions to provide fucosylated products.

For example, the enzyme AcK transfers a phosphate from AcPi to GDP to make GTP and by product Ac (=acetate). Pi both from the PtxD step (in the examples using phosphite) and the PmPpa step will be used by PyrOx.

Lactose, fructose, pyruvate and phosphite are primary reactants in the integrated synthesis. Buffers including, but not limited to, Tris may be used. In some embodiments, the buffer maintains the pH between about pH 6.5 and about 8.5 or about 7.5.

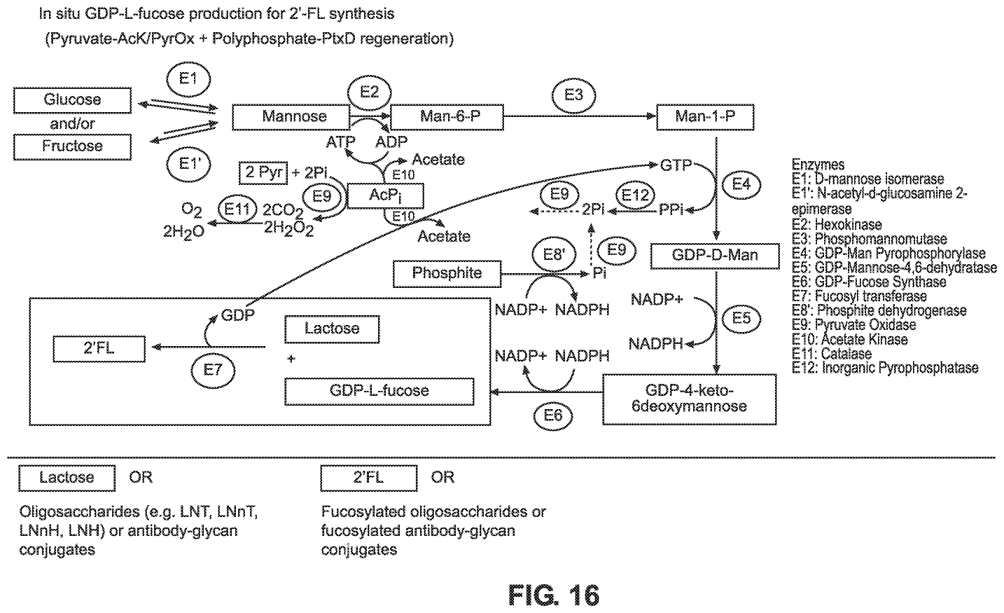

FIG. 16 depicts the same structural transformations of FIG. 15 with a different regeneration system. Here, the regeneration enzymes are E8′, E9, E10, E11, and E12. Here, E8′ converts phosphite to inorganic phosphate and NADP+ to NADPH. Enzymes that may be employed are pyruvate-AcK, PyrOx, and PtxD.

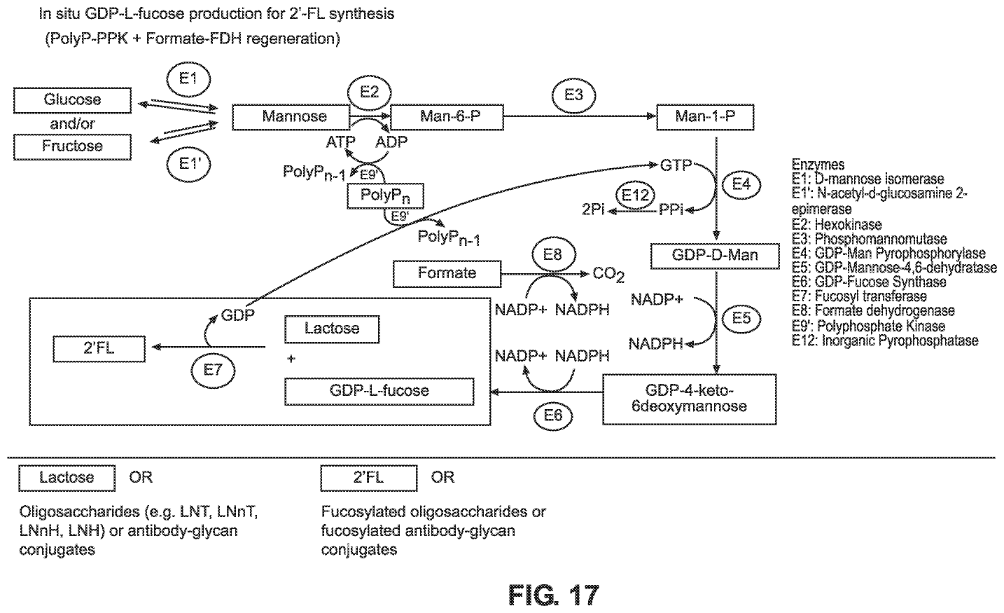

FIG. 17 depicts the same structural transformations of FIG. 15 with a different regeneration system employing E8, E9′, and E12. Here the regeneration enzymes are PolyP-PPK and FDH.

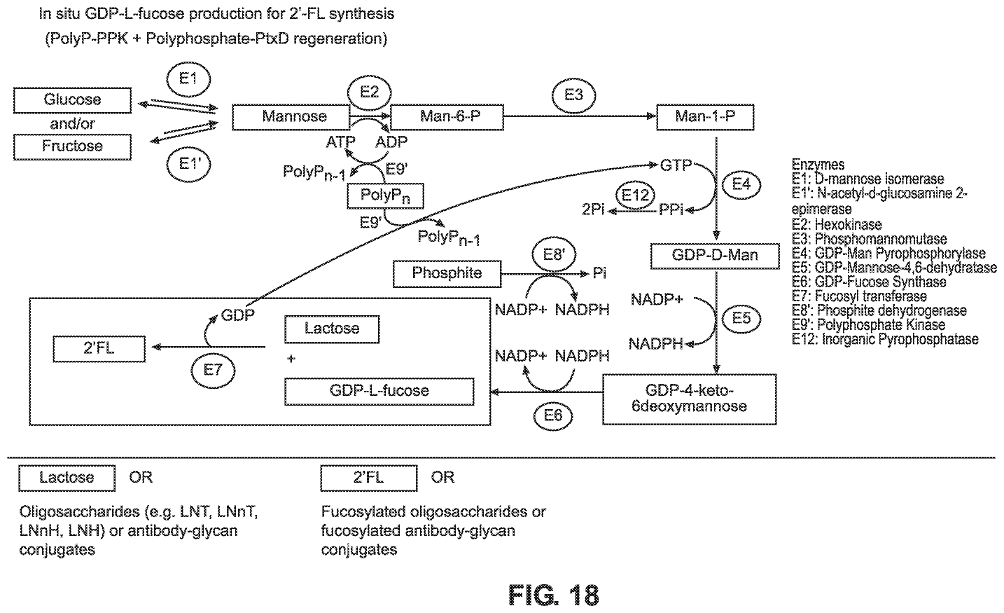

FIG. 18 depicts the same structural transformations of as in FIG. 15, with a different regeneration system. Here, the regeneration enzymes are E8′, E9′, and E12. E9′ is involved in converting ADP to ATP and GDP to GTP, and E8′ converts phosphite to Pi. E12 converts PPi to Pi and converts NADP+ to NADPH. Enzymes that may be used include PolyP-PPK and PtxD.

Feed solutions are also employed in the reactions depicted in FIGS. 15-18.

For example, the feed solution comprises lactose, fructose, formate, MgCl₂, Tris, pyruvate, NADP+, GDP, and ATP (FIG. 15-18).

For example, a feed solution comprises lactose, fructose, phosphite, MgCl₂, a buffer (e.g., Tris buffer), GDP, NADP+, pyruvate, and ATP (FIG. 16).

Alternatively, the feed solution comprises lactose, fructose, formate, MgCl₂, Tris, polyphosphate, a buffer (such as Tris buffer), GDP, NADP+, and ATP (FIG. 17).

Alternatively, the feed solution comprises lactose, fructose, phosphite, MgCl₂, a buffer (such as Tris buffer), GDP, NADP+, polyphosphate, and ATP (FIG. 18).

Glucose or fructose are interchangeable, however the first enzyme must match the starting sugar. Accordingly, either glucose and fructose are interchangeable with the addition of appropriate enzymes E1 or E1′, respectively.

In certain embodiments, compounds that may be fucosylated according to methods of this invention include, but are not limited to, rare sugars, activated sugars, HMOs, and glycans.

Without being bound by theory, manganese (Mn) in combination with high sucrose concentrations leads to enzyme inactivation. In certain embodiments, a process of this invention employs magnesium (Mg) instead of manganese (Mn) and does not include UMP. Under these conditions, high sucrose does not lead to reductive inactivation of GalE.

In certain embodiments the sugars are plant derived. Accordingly, any of the embodiments of this invention may be employed to obtain a compound from non-animal based plant materials. In certain embodiments, the products obtained from the processes of this invention are not animal derived. Such products are advantageous in markets where animal-free products are desired.

Glycans synthesized as described herein may be simple or complex glycan that may be linear or branched. In certain embodiments, the glycans to be fucosylated may be simple glycans or complex glycan, including linear or branched. Glycans having one or more sugar units are included. In certain embodiments, the glycans have two, three, four, or five sugar units or more units. In some embodiments, the glycans have 1-18 units. In some embodiments, the glycans have 1-10 units. In some embodiments, the glycans have 1-5 units. In certain embodiments, the glycans have five sugar units or more. In certain embodiments, the glycans have 1, 2, 3, 4, 5, 6, 7, 8, 9, or 10 units. In certain embodiments, the glycans have 1 unit, 2 units, 3 units, 4 units, 5 units, or 6 units. In some embodiments, the glycans are oligosaccharides. In some embodiments, the glycans are straight chained or branched chained. In certain embodiments, the glycans have 1-6 units and are straight chained. In other embodiments, the glycans have 1-6 units and are branched. In certain embodiments, the glycans have 1-5 units and are straight chained. In other embodiments, the glycans have 1-5 units and are branched. In FIG. 1F n is 3-15. In certain embodiments, n is 3, 4, 5, 6, 7, 8, 9, 10, 11, 12, 13, 14, or 15.

Compounds obtained according to this invention including, but not limited to, galactosylated, sialylated, fucosylated, and N-acetylglucosaminylation compounds, may be used as components in the synthesis of any glycan-containing compound. In certain embodiments, compounds obtained according to this invention include, but are not limited to, galactosylated compounds. The materials functionalized with enzymes, or enzyme systems, have applications for the production of pharmaceuticals, biologicals, nutraceuticals, cosmeceuticals, and food ingredients. In certain embodiments, the sugars and oligosaccharides are non-animal derived.

In one embodiment, fucosylated compounds obtained according to this invention may be used as components in the synthesis of any glycan-containing compound. With the ability to immobilize any enzymes for any processes, the materials functionalized with enzymes, or enzyme systems, have applications for the production of pharmaceuticals, biologicals, actives nutraceutical, actives cosmeceutical and food ingredients.

In certain embodiments, the methods provide fucosylated oligosaccharides or fucosylated antibody-glycan conjugates. In preferred embodiments, fucosylated human milk oligosaccharides (HMOs) are produced.

In certain embodiments, the methods provide galactosylated oligosaccharides or galactosylated antibody-glycan conjugates. In preferred embodiments, galactosylated human milk oligosaccharides (HMOs) are produced.

In certain embodiments, the glycan to be reacted (e.g., galactosylated, sialylated, or fucosylated) is 3′SL, LNTII, LNT, LNnT, LNFPI, LNFPII, LNFPIII, LSTa, LSTb, LSTc, LSTd and DSLNT, LNnH, 3′″₃,3′″₆-di-O-α-Sia-LNnH 3′″₃,3′″₆-di-O-α-Sia-(3″₃,3″₆-di-O-α-Fuc)-LNnH, biantennary sialylated or fucosylated lacto-N-neohexaoses and neoheptaoses, α-2,3-sialyl lacto-N-neopentaose, linear fucosyl- and sialyl-lacto-N-neo-pentaoses, linear lacto-N-neopentaoses, or biantennary lacto-N-neohexaoses and heptaoses.

In certain embodiments, the glycan to be fucosylated is 3′SL, LNTII, LNT, LNnT, LNFPI, LNFPII, LNFPIII, LSTa, LSTb, LSTc, LSTd and DSLNT, LNnH, 3′″₃,3′″₆-di-O-α-Sia-LNnH 3′″₃,3′″₆-di-O-α-Sia-(3″3,3″6-di-O-α-Fuc)-LNnH, biantennary sialylated or fucosylated lacto-N-neohexaoses and neoheptaoses, α-2,3-sialyl lacto-N-neopentaose, linear fucosyl- and sialyl-lacto-N-neo-pentaoses, linear lacto-N-neopentaoses, or biantennary lacto-N-neohexaoses and heptaoses. In certain embodiments, the glycans are 3′SL LNTII, LNT, LNnT, LNFPI, LNFPII, LNFPIII, LSTa, LSTb, LSTc, LSTd, DSLNT, 2′-FL, LNnH, DSLNnH, or DSDFLNNH. In other embodiments, the glycans are 3′SL, LNTII, 2′-FL, LNFPI, LSTa.

In certain embodiments, the glycan to be reacted (e.g., galactosylated) is 2′-FL, 3′SL, LNTII, LNT, LNnT, LNFPI, LNFPII, LNFPIII, LSTa, LSTb, LSTc, LSTd, DSLNT, LNnH, 3′″₃,3′″₆-di-O-α-Sia-LNnH 3′″₃,3′″₆-di-O-α-Sia-(3″₃,3″₆-di-O-α-Fuc)-LNnH, biantennary sialylated or fucosylated lacto-N-neohexaoses and neoheptaoses, α-2,3-sialyl lacto-N-neopentaose, linear fucosyl- and sialyl-lacto-N-neo-pentaoses, linear lacto-N-neopentaoses, or biantennary lacto-N-neohexaoses and heptaoses.

The enzymes used herein may be natural or synthetic, bioengineered enzymes, including fusion enzymes. For example, enzymes may be engineered for affinity towards immobilization scaffolds (tags). Enzymes may also be engineered for improved kinetic properties (e.g., lower Km).

In certain embodiments, the enzymes are immobilized. The immobilization of the enzymes may be through covalent immobilization, entrapment, adsorption, molecular tagging with affinity tags, protein affinity tags, noncovalent adsorption, noncovalent deposition, entrapment, physical entrapment, bioconjugation, chelation, cross-linking, or disulfide bonds. See, e.g., Basso & Serban Mol. Catal. 479 (2019) 110607; Xu et al. Frontiers in Bioeng & Biotech., Published 30 Jun. 2020 publdoi: and Sheldon & van Pelt Chem. Soc. Rev., 2013, 42, 6223. The foregoing are incorporated by reference herein in their entirety.

In embodiments that employ immobilized enzymes, such enzymes include, but are not limited to, enzymes immobilized within bionanocatalysts (BNCs) that in turn are embedded within scaffolds. Bionanocatalysts (BNCs) according to this invention comprise an enzyme self-assembled with magnetic nanoparticles (MNPs). The BNCs self-assemble with the scaffolds.

In some embodiments the immobilized enzymes are non-magnetic. In certain embodiments, such as for oligosaccharides to be used in food ingredients, the immobilized enzymes do not comprise nanoparticles.

In other embodiments, the immobilized enzymes involve permanent molecular entrapment of enzymes within self-assembling nanoparticle (NP) clusters. The self-assembly is purely driven by the materials' electrostatic and magnetic interactions. Ionic strength, buffer pH, and NP concentration are the main parameters impacting the immobilization yield and optimized enzyme activity. The clusters are then magnetically templated onto magnetic scaffolds or shapeable magnetic scaffolds.

In these embodiments, immobilized bionanocatalysts are magnetic materials with one or more enzymes that are immobilized and associated with scaffolds. In some embodiments, the scaffolds are high magnetism and high porosity metal oxides or composite blends of thermoplastics or thermosets comprising magnetic particles that form powders. In some embodiments, Selective Laser Sintering (SLS) is used to design and produce objects via 3D printing by sintering composite magnetic powders.

Certain immobilized enzymes, MNPs, macroporous powders, scaffolds, their structures, organizations, suitable enzymes, and uses are described in WO2012/122437, WO2014/055853, WO2016/186879, WO2017/011292, WO2017/180383, WO2018/34877, WO2018/102319, WO2020/051159, WO2020/69227, as well as WO2022/119982 (U.S. App. Nos. 63/120,669), 63/285,082, and 63/430,271, and 63/448,218. The foregoing are incorporated by reference herein in their entirety.

Bionanocatalysts (BNCs) comprise an enzyme self-assembled with magnetic nanoparticles (MNPs). Self-assembled mesoporous aggregate of magnetic nanoparticles comprise a glycan synthesis enzyme, wherein the mesoporous aggregate is immobilized on a magnetic macroporous scaffold. In one embodiment, the immobilized enzymes comprise (i.) a glycan synthesis enzyme self-assembled in magnetic nanoparticles, and (ii.) a magnetic scaffold. A glycan enzyme is immobilized on nanoparticles where the nanoparticles coat a scaffold, and the enzyme is immobilized in or on the mesoporous structure formed by the nanoparticles.

In one embodiment, the nanoparticles comprise magnetite (Fe₃O₄) or maghemite (Fe₂O₃). In another embodiment, the nanoparticles comprise a product synthesized from FeCl₂ and FeCl₃, particularly synthesized via continuous coprecipitation of FeCl₂*4H₂O and FeCl₃. FeCl₂*4H₂O (Iron (II) chloride tetrahydrate and FeCl₃*6H₂O (Iron (III) chloride hexahydrate. Accordingly, in one embodiment, the nanoparticles comprise magnetite (Fe₃O₄).

A glycan synthesis enzyme is any enzyme that can be used in the synthesis of a glycan. Steps in glycan synthesis may include activating a sugar, transferring a sugar unit thereby extending a sugar, cofactor recycling, and equilibrium shifting. Glycan synthesis enzymes include, but are not limited to, a sugar activation enzyme, a sugar extension enzyme, a reagent regeneration enzyme, and a sugar functionalization enzyme. In another embodiment, glycan synthesis enzymes include, but are not limited to, a sugar activation enzyme, a sugar extension enzyme, a reagent regeneration enzyme, a sugar functionalization enzyme, a sugar support enzyme, a sugar removal enzyme.

Some immobilized enzyme materials, and in particular, magnetic materials, for producing glycans use one or more enzymes that are immobilized within bionanocatalysts (BNCs) which in turn are embedded within macroporous scaffolds to provide scaffolded bionanocatalysts (scaffolded BNCs). The scaffolded BNCs may be inside of modular flow cells for flow manufacturing. The modular flow cells may be mixed and matched for a highly customizable and highly efficient manufacturing processes. The scaffolded BNCs are used in reactions for synthesizing glycans by contacting a glycan subunit or substrate with a scaffolded BNC to produce a second glycan, contacting a first glycan subunit and a second glycan subunit to produce a glycan comprising the first and second glycan subunits, or contacting a first glycan with a scaffolded BNC to produce a second glycan. Included are processes to modifying a glycan subunit and to connect glycans.

Magnetic enzyme immobilization involves the entrapment of enzymes in mesoporous magnetic clusters that self-assemble around the enzymes (level 1). The immobilization efficiency depends on a number of factors that include the initial concentrations of enzymes and nanoparticles, the nature of the enzyme surface, the electrostatic potential of the enzyme, the nature of the nanoparticle surface, and the time of contact. Enzymes used for industrial purposes in biocatalytic processes should be highly efficient, stable before and during the process, reusable over several biocatalytic cycles, and economical.

Mesoporous aggregates of magnetic nanoparticles may be incorporated into continuous or particulate macroporous scaffolds (level 2). The scaffolds may or may not be magnetic. Such scaffolds are discussed in, e.g., WO2014/055853, WO2017/180383, and Corgie et al., Chem. Today 34(5):15-20 (2016), incorporated by reference herein in their entirety. Highly magnetic scaffolds are designed to immobilize, stabilize, and optimize any enzyme. This includes full enzyme systems, at high loading and full activity, and for the production of, e.g., small molecules.

Immobilized enzymes may be employed in process of this invention (WO2022/119982). For example, immobilized enzymes may be prepared as follows. In a first step, strontium ferrite (SFE) scaffold material is coated with magnetic nanoparticles (MNPs) by lowering the pH from 10.0 to 7.5. In a second step, the glycan synthesis enzymes are added to the product from the first step to the scaffolded BNCs. Type B scaffolded BNC compositions are made by this method.

Certain immobilized enzymes are prepared when glycan synthesis enzymes are contacted with magnetic nanoparticles to form a bionanocatalyst (“BNC”) and then the BNCs are contacted with a magnetic scaffold material. Type A scaffolded BNC compositions are made by this method. In certain embodiments, the magnetic scaffold material is strontium ferrite. In one embodiment the strontium ferrite is a spherical particle with a tight size distribution of an average particle diameter of either 20 μm (S20) or 40 μm (S40W; wrinkled). Strontium ferrite in accordance with this invention available upon request from Powdertech International.

Without being bound by theory, combining enzyme(s) and nanoparticles then adding that combination to the scaffold, the enzymes are entrapped (embedded) within the MNPs (Type A). By adding enzyme(s) to nanoparticle coated scaffold a Type B composition is obtained, wherein the enzymes remain more exposed at the surface and are not buried as much. As used herein, scaffold-MNP complex, scaffold-MNP matrix, and scaffold-MNP material each indicate the combination of a scaffold and a MNP according to this invention, comprising, consisting essentially of, or consisting of, magnetite nanoparticles and a strontium ferrite matrix.

In certain embodiments, this invention employs enzymes immobilized using iron oxide materials including, but not limited to, hematite, magnetite, and strontium ferrite. In certain embodiments the immobilized enzyme is a Type A scaffolded BNC or a Type B scaffolded BNC.

Accordingly, this invention provides a glycan synthesis enzyme scaffolded BNC made by the process of contacting strontium ferrite with magnetite nanoparticles to form a scaffold-MNP Complex and adding a glycan synthesis enzyme to the scaffold-MNP Complex to form the scaffolded BNC. Another embodiment provides a glycan synthesis enzyme scaffolded BNC made by the process of combining magnetite nanoparticles and a glycan synthesis enzyme to form a BNC and then contacting the BNC with a scaffold or matrix to form a scaffolded BNC. Without being bound by theory, the bionanocatalyst coats the magnetic microporous scaffold material.

One embodiment of this invention a scaffolded BNC composition comprises a self-assembled mesoporous aggregate of magnetic nanoparticles and a glycan synthesis enzyme and a magnetic microporous material. A scaffolded BNC according to this invention comprises a glycan synthesis enzyme immobilized on magnetic nanoparticles, wherein the magnetic nanoparticles coat a magnetic macroporous material. In certain embodiments, the scaffolded BNCs comprises a magnetic macroporous matrix material comprising self-assembled mesoporous aggregates of magnetic nanoparticles magnetically entrapping an immobilized glycan synthesis enzyme In certain embodiments, the scaffolded BNCs consists of, or consists essentially of, a magnetic macroporous matrix material comprising self-assembled mesoporous aggregates of magnetic nanoparticles magnetically entrapping an immobilized glycan synthesis enzyme. In any embodiments, the scaffolded BNC comprises any elementary enzyme module described herein or is in a system module.

A scaffolded BNC composition comprises a self-assembled mesoporous aggregate of magnetic nanoparticles and a glycan synthesis enzyme and a magnetic microporous material. A scaffolded BNC that may be used in the methods of this invention comprises a glycan synthesis enzyme immobilized on magnetic nanoparticles, wherein the magnetic nanoparticles coat a magnetic macroporous material. In certain embodiments, the scaffolded BNCs consist of a magnetic macroporous matrix material comprising self-assembled mesoporous aggregates of magnetic nanoparticles magnetically entrapping an immobilized glycan synthesis enzyme. In certain embodiments, the scaffolded BNC is used in an integrated pathway as disclosed herein.

The invention provides for the ability to perform syntheses in one-pot reactions. The one-pot synthesis may be done in batch or flow including, but not limited to, repetitive batch or continuous flow. In a flow reactor, such as a packed bed reactor, the mixture of reagents is passed through the flow. Thus, in one embodiment, the invention provides a commercially applicable biocatalysis in flow.

The immobilized enzymes provide a series of highly tunable materials and processes for universal enzyme immobilization based on magnetic metamaterials. The unique enzyme hierarchical immobilization platform provides optimal conditions to immobilize single and full systems of enzymes and allows optimal conditions to be found and adapted for single and full systems of enzymes. It affords enzyme stability, maximal use of substrates (including co-factors) and imparts modularity to flow processes. Accordingly, certain methods of this invention use a stabilized enzyme composition comprising a bionanocatalyst and a magnetic scaffold, wherein the bionanocatalyst comprises a glycan synthesis enzyme and magnetic nanoparticles and the magnetic scaffold stabilizes the bionanocatalyst.

One embodiment provides a modular process for producing a glycan, comprising a module that may be a flow cell wherein: the module comprises a magnetic macroporous powder comprising magnetic microparticles, wherein the powder has immobilized a preparation of self-assembled mesoporous aggregates of magnetic nanoparticles containing a glycan synthesis enzyme; wherein a substrate is introduced into the module (or passed through a flow cell) and the substrate is modified to provide a glycan.

Continuous flow reactors include, but are not limited to, packed-bed and fixed-bed reactors in tubular format can be combined with upstream and downstream processes that are not continuous making the overall process semi-continuous. For example, the reaction feed for the LNTII reaction (Example 3c-A) may be produced in a continuous stirred tank reactor, the product of which is continuously added to the flow reactor. Microfluidic reactors may also be employed in connection with this invention.

Certain embodiments involve methods comprising 1 or more modules. In a method comprising more than a first module, a first substrate is passed through the first modular flow cell to create a modified substrate; wherein the modified substrate is a second substrate to pass through a second module to create a second modified substrate. The invention provides for sets of modules to be combined allowing the synthesis of complex glycans.

In one embodiment, the invention provides methods for making glycans by immobilizing an enzyme with magnetic nanoparticles and contacting the immobilized enzyme with appropriate synthetic reagents. The methods may be conducted in batch, flow, semi-continuous, or continuous-flow.

Scaffolds according to the invention are chemically inert, structurally tunable to fit any process, and highly magnetic to ensure full capture of the enzyme-containing cluster. In one embodiment, the magnetic macroporous material comprises a metal oxide or a metal oxide complex. In one embodiment, the scaffold comprises a metal oxide. In one embodiment, the metal oxide is strontium ferrite (SrFe₁₂O₁₉). In certain embodiments, the magnetic macroporous material is a metal oxide and consists essentially of, or consists of, metallic materials or ceramic and does not include a polymer. In certain embodiments, the scaffold is a metal oxide and is not a nanoparticle.

The invention also provides a process for preparing a scaffolded bionanocatalyst by combining a magnetic nanoparticle and a glycan synthesis enzyme to form a bionanocatalyst and then contacting the bionanocatalyst with a scaffold to obtain the scaffolded bionanocatalyst, and a process for preparing a scaffolded bionanocatalyst by combining a scaffold and a magnetic nanoparticle to form a scaffolded magnetic nanoparticle complex and then contacting the scaffolded magnetic nanoparticle complex with a glycan synthesis enzyme. Also provided is a scaffolded BNC made by either of these processes.

The process for preparing the magnetic scaffolds is flexible as employed in this invention provides for convenient, flexible glycosylation reactions.

In one embodiment, the process for preparing the magnetic scaffolds is flexible as employed in this invention provides for convenient, flexible fucosylation reactions.

The process for preparing the magnetic scaffolds is flexible and tunable to manufacture objects using 3D designs that magnetically capture the BNCs. A large surface area may result from the sintering process itself. Materials can also be recycled by removing the BNCs and then re-functionalized them for repeated use. See PCT/US19/53307, incorporated by reference herein in its entirety.

In some embodiments, thermoplastics are Polyethylene (PE) (varying densities, e.g. LDPE, HDPE), Polypropylene (PP), Acrylics: Polyacrylic acids (PAA), Poly(methyl methacrylate) (PMMA), Polyvinyl alcohol (and polyvinyl acetals), Polyamides (Nylon), Polylactic acid (PLA), Polycarbonate (PC), Polyether sulfone (PES), Polystyrene (PS), Polyvinyl chloride (PVC), Acrylonitrile butadiene styrene (ABS), Polybenzimidazole (PBI), Polyoxymethylene (POM), Polyetherether ketone (PEEK), Polyetherimide (PEI), Polyvinylidene fluoride (PVDF), Polytetrafluoroethylene (PTFE/Teflon), Polyacrylonitrile (PAN)) blended with magnetic materials (e.g. magnetite MMP) via melting/extrusion or via coating of the magnetic material by dissolving the plastic in a solvent. In other embodiments, the powders are sintered by a laser using SLS. Porosity may be formed during SLS.

Selective laser sintering (SLS) is an additive manufacturing (AM) technique that uses a laser as the power source to sinter powdered materials such as plastic, metal, ceramic, glass powders, nylon or polyamide. A laser automatically aimed at points in space, defined by a 3D model (e.g. an Additive Manufacturing File, AMF, or a CAD file), binds the material together to create a solid structure. After each cross-section is scanned, the powder bed is lowered by a one-layer thickness, a new layer of material is applied on top, and the process is repeated until the part is completed. There are many different technologies, such as stereolithography (SLA) or fused deposit modeling (FDM).

SLS is similar to direct metal laser sintering (DMLS) but differs in technical details. DSLM uses a comparable concept, but in DSLM the material is fully melted rather than sintered. This allows one to manufacture materials with different properties (e.g. crystal structure and porosity). SLS is a relatively new technology that may be expanded into commercial-scale manufacturing processes.

In one embodiment, polypropylene-magnetite materials can be 3D-printed in any shape and form via SLS.

In other embodiments, an extruded composite material is size reduced via cryomilling or another form of milling. In other embodiments, composite powders are sieved to an ideal particle size. In preferred embodiments, the particle sizes are 60+/−20 μm.

Powders or 3D printed objects can be functionalized with BNCs containing one or more enzymes or enzyme systems. BNCs are magnetically trapped at the surface of the powders or 3D printed objects.

They may be single-layered or multiple-layered materials that hold the BNCs. Such designed objects may be produced using 3D printing by sintering composite magnetic powders. In some embodiments, Selective Laser Sintering (SLS) is used. The modular flow cells may be mixed and matched for a highly customizable and highly efficient manufacturing process.

Composite powders may also be optimized for flowability. In some embodiments, 3D objects can be printed to optimize flow within to be used in flow reactors.

In some embodiments, 3D objects and composite powders can be washed from the BNCs by an acid wash, rinsed with water, and then re-functionalized with fresh BNCs.

Highly magnetic scaffolds (Macroporous Magnetic Scaffolds or MMP) are designed to immobilize, stabilize and optimize any BNCs containing enzymes. This includes full enzyme systems at high loading and full activity for the production of small molecules. By combining natural or engineered enzymes, and in some embodiments with cofactor recycling systems, the scaffolds allow one to scale up biocatalysis to innovations to manufacturing scale and production.

Included are highly magnetic and highly porous composite blends of thermoplastics with magnetic particles to form powders that may be single-layered or multiple-layered materials that hold the BNCs. Such designed objects may be produced using 3D printing by sintering composite magnetic powders. In some embodiments, Selective Laser Sintering (SLS) is used. The modular flow cells may be mixed and matched for a highly customizable and highly efficient manufacturing process.

MMP made of thermoplastic and magnetic materials of the invention can take the form of magnetic powders that are suitable for flow chemistry application. These powders can be 3D printed by SLS as structures, as functional objects, or as flow cells or plate reactors. High surface areas allow one to maximize the enzyme loading and flow can be engineered within the materials to enable biocatalysis at maximal productivity.

SLS can be used to process nearly any kind of material from metals, ceramics, plastics, and combinations thereof, for tailor-made composite materials. It is critical, however, that the material is available in fine powder form and that the powder particles are operative to fuse when exposing them to heat (Kruth et al., Assembly Automation 23(4):357-371(2003), incorporated by reference herein in its entirety.

When the material lacks those features or is prone to phase transitions at the temperature range or conditions of the sintering process, the addition of a sacrificial binder can make this process still feasible for that material. Commonly, polymers are used as sacrificial binders in order to expand the range of materials suitable for this technology. After sintering, the sacrificial binder can be either removed by thermal decomposition or kept as part of the composition.

This concept applies to magnetite that loses its permanent magnetic properties above 585° C. This is significantly lower than its melting temperature (1538° C.). Another advantage of using a polymeric matrix to incorporate magnetite particles is that the former can act as a protective barrier to prevent oxidation and corrosion as well as aiding to disperse the magnetite particles. Also, magnetite can mechanically reinforce the polymer. (Shishkovsky et al., Microelectronic Engineering 146:85-91 (2015), incorporated by reference herein in its entirety).

Laser sintering of plastic parts is one of two additive manufacturing processes used for Rapid Manufacturing (Wegner, Physics Procedia 83:1003-1012 (2016), incorporated by reference herein in its entirety). There are several polymer properties that determine its capability to be sintered and produce good quality 3D objects. These include structural properties such crystalline structure (i.e. thermal properties such as Tm, Tg, and Tc), mechanical properties (Young's modulus and elongation at break, etc.), density, particle size, and shape.

In SLS, the temperature-processing window is determined from the difference between the melting and crystallization temperatures of the polymer. For instance, nylon 12 (PA 12) has one of the highest operational windows and is thus a widely used SLS material. In theory, the higher this value is, the easier the material can be sintered. In practice, however there are many more parameters that can still make this process difficult for any specific polymer (Shishkovsky et al., Microelectronic Engineering 146:85-91 (2015)), incorporated by reference herein in its entirety). In order to prevent curling of the sintered part, a low polymer crystallization rate is desired together with a melt index that provides a suitable rheology and surface tension.

Additionally, the bulk density, particle shape, and size distribution of the powder are key factors (Wegner, Physics Procedia 83:1003-1012 (2016), incorporated by reference herein in its entirety). It has been determined that the in certain embodiments, the optimal particle size range is about 40 to about 90 microns. Smaller particles prevent flowability and their rapid vaporization is detrimental to the optical sensors of the sintering device. This can fog the device and lead to inaccurately sintered parts (Goodridge et al. Materials Science 57:229-267 (2012), incorporated by reference herein in its entirety). The powders should have good flowing properties and preferably an approximately round particle shape. This allows good powder spreading during the process. High heat conductivity of the material is desired at the CO₂ laser beam wavelength (10.6 microns). This is not the case for most polymers. The last two requirements can be met by the incorporation of additives such as high-energy absorption materials, e.g. carbon black, to improve heat absorption, and fume silica nanoparticles (talc) to aid the particle flowability with irregularly-shaped particles.

Additive manufacturing (AM), also referred to as 3D printing, involves manufacturing a part by depositing material layer-by-layer. This differs from conventional processes such as subtractive processes (i.e., milling or drilling), formative processes (i.e., casting or forging), and joining processes (i.e., welding or fastening). Quick production time, low prototyping costs, and design flexibility make 3D printing a valuable tool for both prototyping and industrial manufacturing. The three most common types of 3D printers are fused filament fabrication, stereolithography, and selective laser sintering.

Fused filament fabrication (FFF) melts a thermoplastic continuous filament and builds the object layer by layer until the print is complete. Although alternative materials exist, the two most popular filament materials are polylactic acid (PLA) and acrylonitrile butadiene styrene (ABS). FFF printers and materials are among the cheapest on the market but currently have a lower print resolution and build quality.

Stereolithography (SLA) uses a laser to polymerize photosensitive resins. Uncured liquid resin is placed in a vat where a laser is used to cure resin into solid plastic and build the object layer by layer. SLA printers have a much higher resolution than FFF printers due to the fine spot size of the laser and thus can print intricate features and complex shapes. The resins, however, are more expensive than filaments and completed prints currently require post processing with solvents to optimize the surface finish and material characteristics.

Selective laser sintering (SLS) is a powder-based layer-additive manufacturing process generally meant for rapid prototyping and rapid tooling. Laser beams either in continuous or pulse mode are used as a heat source for scanning and joining powders in predetermined sizes and shapes of layers. The geometry of the scanned layers corresponds to the various cross sections of the computer-aided design (CAD) models or stereolithography (STL) files of the object. After the first layer is scanned, a second layer of loose powder is deposited over it, and the process is repeated from bottom to top until the artifact (3D object) is complete.” Kumar, JOM, 55(10), 43-47 (2003), incorporated by reference herein in its entirety.

SLS provides advantages for printing objects with magnetic properties that can be used for immobilizing BNCs. This is because the printing process creates porosity and a high surface area. The surrounding, unsintered powder acts as a natural support that eliminates the need for dedicated support structures. The lack of support structures allows for complex geometries that would otherwise be impossible to manufacture using alternative 3D printing methods. In addition, the nature of sintering itself creates macro and microporous volumes. During the printing process, the laser flashes thermoplastic crystalline thermoplastic powders (e.g. Polypropylene, polystyrene) between their glass transition temperature and melting temperature to generate stiff parts. By avoiding amorphous behavior with a quick laser scan speed (>100 mm/s), powders are sintered in place to form small bonds amongst themselves. The low-density powders trap air in their structures resulting in remarkable porosity and surface area in three dimensions. These pores increase the surface area for enzyme immobilization.

In recent years, industrial use of enzymes has garnered significant attention due to the wide range of potential manufacturing applications. Using enzymes in industrial processes offers several advantages over conventional chemical methods. This includes high catalytic activity, the ability to perform complex reactions, and promoting greener chemistry by reducing by-products and the need for toxic chemicals (Singh et al., Microbial enzymes: industrial progress in 21st century. 3 Biotech. 6(2):174 (2016), incorporated by reference herein in its entirety).

One of the biggest hindrances to widespread biocatalysis use in industrial production is low enzyme stability. This is further hampered by relatively harsh process conditions that can destabilize enzymes and decrease their lifespan (Mohamad et al., Biotechnology, Biotechnological Equipment 29(2):205-220 (2015), incorporated by reference herein in its entirety). Furthermore, the use of free enzymes in these processes are generally lost from the system as waste products and therefore become a costly operating cost. The primary solution to these issues is immobilization of enzymes onto scaffolding to enhance their operational stability and catalytic activity. Enzyme immobilization also provides a method for enzyme recovery, making biocatalytic processes more economically feasible.

Currently, biocatalytic processes for industrial production are generally carried out in batch reactors due to their simplicity and ease of operation. Despite the benefits of using batch reactors, continuous flow systems enable higher productivity and better process control (Wiles C et al., Green Chem. (14):38-54 (2012)). The rapid development of flow chemistry in biocatalytic processes has primarily been driven by a growing interest in process intensification and green chemistry. Continuous flow systems facilitate process intensification by decreasing residence times (often from hours to minutes), reducing the size of equipment required, and enabling production volume enhancement (Tamborini et al., Cell. 36(1):73-78 (2018)). From a green chemistry standpoint, these systems offer significant improvements in safety, waste generation, and energy efficiency due to heat management and mixing control (Newman and Jensen, Green Chem. (15):1456-1472 (2013)). The foregoing are incorporated by reference herein in their entirety.

The invention has many benefits over the prior art. It enables the efficient and economical production of glycans, such as complex polysaccharides, including but not limited to, HMOs using enzymes captured in modular flow processing cells. The flow cells may contain materials having large macropores or a high magnetic surface area for BNC immobilization. Flexible compositions for sintered magnetic scaffolds can be made with any meltable thermoplastics and magnetic material composition. The flow cells can have one or multiple enzyme systems that may be pieced together for particular sugar manufacturing processes.

A solution to combining biocatalysis and continuous flow systems is with functionalized flow cells. Biocatalytic flow cells are scaffolds containing immobilized enzymes for use in reactors such as continuous stirred tank reactors (CSTRs) and packed bed reactors (PBRs). Both types of reactors are known in the art but are primarily chosen based on the type of immobilization used. With a total market value of $5.8B in 2010, immobilized enzymes are used in a diverse range of large-scale processes including high fructose corn syrup production (10⁷ tons/year), transesterification of food oils (10⁵ tons/year), biodiesel synthesis (10⁴ tons/year), and chiral resolution of alcohols and amines (10³ tons/year) (DiCosimo et al., Chem. Soc. Rev. (42):6437-6474 (2013), incorporated by reference herein in its entirety). These systems allow for improved downstream process management for enzymatic systems compared to batch reactors in terms of in-line control, enzyme reuse, and production scalability.

For the foregoing reasons, the methods of this invention employ biocatalytic systems for small-to-large scale manufacturing using BNCs in scaffolds that are shaped by 3D printing. In some embodiments, the biocatalytic systems are continuous flow.

Scaffolds may comprise cross-linked water-insoluble polymers and an approximately uniform distribution of embedded magnetic microparticles (MMP). The scaffolds may contain thermoset resins including Epoxy resins, Polyesters, Polyurethanes, Melamine resins, Vinyl esters, Silicones (polysiloxanes), Furan resins, Polyurea, Phenolic resins, phenol-formaldehyde, Urea-formaldehyde, Diallyl-phthalate (DAP), Benzoxazine, Polyimides and bismaleimides, Cyanate esters can be used. By combining natural or engineered enzymes, and in some embodiments with cofactors and cofactor recycling systems, the scaffold technology disclosed herein allows one to quickly translate innovation in biocatalysis to innovation in production for batch and flow processes. The magnetic powders are suitable for use flow chemistry applications such as pack-bed reactors.

Self-assembled mesoporous nanoclusters comprising entrapped enzymes are highly active and robust. The technology is a powerful blend of biochemistry, nanotechnology, and bioengineering at three integrated levels of organization: Level 1 is the self-assembly of enzymes with magnetic nanoparticles (MNP) for the synthesis of magnetic mesoporous nanoclusters. This level uses a mechanism of molecular self-entrapment to immobilize and stabilize enzymes. Level 2 is the stabilization of the MNPs into other matrices. Level 3 is product conditioning and packaging for Level 1+2 delivery. The assembly of magnetic nanoparticles adsorbed to enzyme is herein also referred to as a “bionanocatalyst” (BNC).

MNPs allow for a broader range of operating conditions such as temperature, ionic strength and pH. The size and magnetization of the MNPs affect the formation and structure of the NPs, all of which have a significant impact on the activity of the entrapped enzymes. By virtue of their surprising resilience under various reaction conditions, MNPs can be used as improved enzymatic or catalytic agents where other such agents are currently used. Furthermore, they can be used in other applications where enzymes have not yet been considered or found applicable.

The BNC contains mesopores that are interstitial spaces between the magnetic nanoparticles. The enzymes are preferably embedded or immobilized within at least a portion of mesopores of the BNC. As used herein, the term “magnetic” encompasses all types of useful magnetic characteristics, including permanent magnetic, superparamagnetic, paramagnetic, ferromagnetic, and ferrimagnetic behaviors.

The magnetic nanoparticle or BNC has a size in the nanoscale, i.e., generally no more than 500 nm. As used herein, the term “size” can refer to a diameter of the magnetic nanoparticle when the magnetic nanoparticle is approximately or substantially spherical. In a case where the magnetic nanoparticle is not approximately or substantially spherical (e.g., substantially ovoid or irregular), the term “size” can refer to either the longest the dimension or an average of the three dimensions of the magnetic nanoparticle. The term “size” may also refer to an average of sizes over a population of magnetic nanoparticles (i.e., “average size”).

In different embodiments, the magnetic nanoparticle has a size of precisely, about, up to, or less than, for example, 500 nm, 400 nm, 300 nm, 200 nm, 100 nm, 50 nm, 40 nm, 30 nm, 25 nm, 20 nm, 15 nm, 10 nm, 5 nm, 4 nm, 3 nm, 2 nm, or 1 nm, or a size within a range bounded by any two of the foregoing exemplary sizes.

In the BNC, the individual magnetic nanoparticles can be considered to be primary nanoparticles (i.e., primary crystallites) having any of the sizes provided above. The aggregates of nanoparticles in a BNC are larger in size than the nanoparticles and generally have a size (i.e., secondary size) of at least about 5 nm. In different embodiments, the aggregates have a size of precisely, about, at least, above, up to, or less than, for example, 5 nm, 8 nm, 10 nm, 12 nm, 15 nm, 20 nm, 25 nm, 30 nm, 35 nm, 40 nm, 45 nm, 50 nm, 60 nm, 70 nm, 80 nm, 90 nm, 100 nm, 150 nm, 200 nm, 300 nm, 400 nm, 500 nm, 600 nm, 700 nm, or 800 nm, or a size within a range bounded by any two of the foregoing exemplary sizes.

Typically, the primary and/or aggregated magnetic nanoparticles or BNCs thereof have a distribution of sizes, i.e., they are generally dispersed in size, either narrowly or broadly dispersed. In different embodiments, any range of primary or aggregate sizes can constitute a major or minor proportion of the total range of primary or aggregate sizes. For example, in some embodiments, a particular range of primary particle sizes (for example, at least about 1, 2, 3, 5, or 10 nm and up to about 15, 20, 25, 30, 35, 40, 45, or 50 nm) or a particular range of aggregate particle sizes (for example, at least about 5, 10, 15, or 20 nm and up to about 50, 100, 150, 200, 250, or 300 nm) constitutes at least or above about 50%, 60%, 70%, 80%, 90%, 95%, 98%, 99%, or 100% of the total range of primary particle sizes. In other embodiments, a particular range of primary particle sizes (for example, less than about 1, 2, 3, 5, or 10 nm, or above about 15, 20, 25, 30, 35, 40, 45, or 50 nm) or a particular range of aggregate particle sizes (for example, less than about 20, 10, or 5 nm, or above about 25, 50, 100, 150, 200, 250, or 300 nm) constitutes no more than or less than about 50%, 40%, 30%, 20%, 10%, 5%, 2%, 1%, 0.5%, or 0.1% of the total range of primary particle sizes.

The aggregates of magnetic nanoparticles (i.e., “aggregates”) or BNCs thereof can have any degree of porosity, including a substantial lack of porosity depending upon the quantity of individual primary crystallites they are made of. In particular embodiments, the aggregates are mesoporous by containing interstitial mesopores (i.e., mesopores located between primary magnetic nanoparticles, formed by packing arrangements). The mesopores are generally at least 2 nm and up to 50 nm in size. In different embodiments, the mesopores can have a pore size of precisely or about, for example, 2, 3, 4, 5, 10, 12, 15, 20, 25, 30, 35, 40, 45, or 50 nm, or a pore size within a range bounded by any two of the foregoing exemplary pore sizes. Similar to the case of particle sizes, the mesopores typically have a distribution of sizes, i.e., they are generally dispersed in size, either narrowly or broadly dispersed. In different embodiments, any range of mesopore sizes can constitute a major or minor proportion of the total range of mesopore sizes or of the total pore volume. For example, in some embodiments, a particular range of mesopore sizes (for example, at least about 2, 3, or 5, and up to 8, 10, 15, 20, 25, or 30 nm) constitutes at least or above about 50%, 60%, 70%, 80%, 90%, 95%, 98%, 99%, or 100% of the total range of mesopore sizes or of the total pore volume. In other embodiments, a particular range of mesopore sizes (for example, less than about 2, 3, 4, or 5 nm, or above about 10, 15, 20, 25, 30, 35, 40, 45, or 50 nm) constitutes no more than or less than about 50%, 40%, 30%, 20%, 10%, 5%, 2%, 1%, 0.5%, or 0.1% of the total range of mesopore sizes or of the total pore volume.

The magnetic nanoparticles can have any of the compositions known in the art. In some embodiments, the magnetic nanoparticles are or include a zerovalent metallic portion that is magnetic. Some examples of such zerovalent metals include cobalt, nickel, and iron, and their mixtures and alloys. In other embodiments, the magnetic nanoparticles are or include an oxide of a magnetic metal, such as an oxide of cobalt, nickel, or iron, or a mixture thereof. In some embodiments, the magnetic nanoparticles possess distinct core and surface portions. For example, the magnetic nanoparticles may have a core portion composed of elemental iron, cobalt, or nickel and a surface portion composed of a passivating layer, such as a metal oxide or a noble metal coating, such as a layer of gold, platinum, palladium, or silver. In other embodiments, metal oxide magnetic nanoparticles or aggregates thereof are coated with a layer of a noble metal coating. The noble metal coating may, for example, reduce the number of charges on the magnetic nanoparticle surface, which may beneficially increase dispersibility in solution and better control the size of the BNCs. The noble metal coating protects the magnetic nanoparticles against oxidation, solubilization by leaching or by chelation when chelating organic acids, such as citrate, malonate, or tartrate are used in the biochemical reactions or processes. The passivating layer can have any suitable thickness, and particularly, at least, up to, or less than, about for example, 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or 10 nm, or a thickness in a range bounded by any two of these values.

Magnetic materials useful in the invention are well-known in the art. Non-limiting examples comprise ferromagnetic and ferromagnetic materials including ores such as iron ore (magnetite or lodestone), cobalt, and nickel. In other embodiments, rare earth magnets are used. Non-limiting examples include neodymium, gadolinium, sysprosium, samarium-cobalt, neodymium-iron-boron, and the like. In yet further embodiments, the magnets comprise composite materials. Non-limiting examples include ceramic, ferrite, and alnico magnets. In preferred embodiments, the magnetic nanoparticles have an iron oxide composition. The iron oxide composition can be any of the magnetic or superparamagnetic iron oxide compositions known in the art, e.g., magnetite (Fe₃O₄), hematite (α-Fe₂O₃), maghemite (γ-Fe₂O₃), or a spinel ferrite according to the formula AB₂O₄, wherein A is a divalent metal (e.g., Xn²+, Ni²+, Mn²⁺, Co²⁺, Ba²⁺, Sr²⁺, or combination thereof) and B is a trivalent metal (e.g., Fe³⁺, Cr³⁺, or combination thereof).

The individual magnetic nanoparticles or aggregates thereof or BNCs thereof possess any suitable degree of magnetism. For example, the magnetic nanoparticles, BNCs, or BNC scaffold assemblies can possess a saturated magnetization (Ms) of at least or up to about 5, 10, 15, 20, 25, 30, 40, 45, 50, 60, 70, 80, 90, or 100 emu/g. The magnetic nanoparticles, BNCs, or BNC-scaffold assemblies preferably possess a permanent magnetization (Mr) of no more than (i.e., up to) or less than 5 emu/g, and more preferably, up to or less than 4 emu/g, 3 emu/g, 2 emu/g, 1 emu/g, 0.5 emu/g, or 0.1 emu/g. The surface magnetic field of the magnetic nanoparticles, BNCs, or BNC-scaffold assemblies can be about or at least, for example, about 0.5, 1, 5, 10, 50, 100, 200, 300, 400, 500, 600, 700, 800, 900, or 1000 Gauss (G), or a magnetic field within a range bounded by any two of the foregoing values. If microparticles are included, the microparticles may also possess any of the above magnetic strengths.

The magnetic nanoparticles or aggregates thereof can be made to adsorb a suitable amount of enzyme, up to or below a saturation level, depending on the application, to produce the resulting BNC. In different embodiments, the magnetic nanoparticles or aggregates thereof may adsorb about, at least, up to, or less than, for example, 1, 5, 10, 15, 20, 25, or 30 pmol/m2 of enzyme. Alternatively, the magnetic nanoparticles or aggregates thereof may adsorb an amount of enzyme that is about, at least, up to, or less than, for example, about 10%, 20%, 30%, 40%, 50%, 60%, 70%, 80%, 90%, or 100% of a saturation level.

The magnetic nanoparticles or aggregates thereof or BNCs thereof possess any suitable pore volume. For example, the magnetic nanoparticles or aggregates thereof can possess a pore volume of about, at least, up to, or less than, for example, about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 cm3/g, or a pore volume within a range bounded by any two of the foregoing values.

The magnetic nanoparticles or aggregates thereof or BNCs thereof possess any suitable specific surface area. For example, the magnetic nanoparticles or aggregates thereof can have a specific surface area of about, at least, up to, or less than, for example, about 50, 60, 70, 80, 90, 100, 110, 120, 130, 140, 150, 160, 170, 180, 190, or 200 m 2/g.

The magnetic macroporous matrix material for use according to this invention has a size of precisely, about, up to, or less than, for example, 100-1000, 50-100, 10-50 μm, or 5-10, 1000, 900, 800, 700, 600, 500, 400, 300, 200, 100, 90, 80, 70, 60, 50, 40, 30, 20, 10, 5, less than 5, greater than 100, an average size of 150, an average size of 75, an average size of 40, an average size of 20 or an average size of about 15. In certain embodiments, the material has an average particle diameter of precisely, about, up to, or less than, 20-40 μm, 20 μm, or 40 μm. In certain embodiments, the material has a tight size distribution of an average particle diameter of either 20 μm or 40 μm.

In some embodiments, the methods described herein use recombinant cells that express the enzymes used in the invention. Recombinant DNA technology is known in the art. In some embodiments, cells are transformed with expression vectors such as plasmids that express the enzymes. In other embodiments, the vectors have one or more genetic signals, e.g., for transcriptional initiation, transcriptional termination, translational initiation and translational termination. Here, nucleic acids encoding the enzymes may be cloned in a vector so that they are expressed when properly transformed into a suitable host organism. Suitable host cells may be derived from bacteria, fungi, plants, or animals as is well-known in the art.

Although BNCs (Level 1) provide the bulk of enzyme immobilization capability, they are sometimes too small to be easily captured by standard-strength magnets. Thus, sub-micrometric magnetic materials (Level 2) are used to provide bulk magnetization and added stability to Level 1. Commercially available free magnetite powder, with particle sizes ranging from 50-500 nm, is highly hydrophilic and tends to stick to plastic and metallic surfaces, which, over time, reduces the effective amount of enzyme in a given reactor system. In addition, powdered magnetite is extremely dense, thus driving up shipping costs. It is also rather expensive—especially at particle sizes finer than 100 nm. To overcome these limitations, low-density hybrid materials consisting of magnetite, non-water-soluble cross-linked polymers such as poly(vinylalcohol) (PVA) and carboxymethylcellulose (CMC), have been developed. These materials are formed by freeze-casting and freeze-drying water-soluble polymers followed by cross-linking. These materials have reduced adhesion to external surfaces, require less magnetite, and achieve Level 1 capture that is at least comparable to that of pure magnetite powder.

In one embodiment, the continuous macroporous scaffold has a cross-linked polymeric composition. The polymeric composition can be any of the solid organic, inorganic, or hybrid organic-inorganic polymer compositions known in the art, and may be synthetic or a biopolymer that acts as a binder. Preferably, the polymeric s scaffold does not dissolve or degrade in water or other medium in which the hierarchical catalyst is intended to be used. Some examples of synthetic organic polymers include the vinyl addition polymers (e.g., polyethylene, polypropylene, polystyrene, polyacrylic acid or polyacrylate salt, polymethacrylic acid or polymethacrylate salt, poly(methylmethacrylate), polyvinyl acetate, polyvinyl alcohol, and the like), fluoropolymers (e.g., polyvinylfluoride, polyvinylidenefluoride, polytetrafluoroethylene, and the like), the epoxides (e.g., phenolic resins, resorcinol-formaldehyde resins), the polyamides, the polyurethanes, the polyesters, the polyimides, the polybenzimidazoles, and copolymers thereof. Some examples of biopolymers include the polysaccharides (e.g., cellulose, hemicellulose, xylan, chitosan, inulin, dextran, agarose, and alginic acid), polylactic acid, and polyglycolic acid. In the particular case of cellulose, the cellulose may be microbial- or algae-derived cellulose. Some examples of inorganic or hybrid organic-inorganic polymers include the polysiloxanes (e.g., as prepared by sol gel synthesis, such as polydimethylsiloxane) and polyphosphazenes. In some embodiments, any one or more classes or specific types of polymer compositions provided above are excluded as macroporous scaffolds. In some embodiments the 3D model is an electronic file.

Any of these compositions and methods may be used in the embodiments of this invention to immobilize an enzyme.

It should be understood that ‘comprise’ is, where context permits, to be interpreted non-exhaustively. Where context permits, each comprise is alternatively “consist essentially of”, or “consist of”

### Embodiments

1. A biocatalytic process for preparing a saccharide-acceptor comprising the steps of:
a. contacting a saccharide and a nucleotide to form a saccharide-nucleotide, wherein the nucleotide is present in a catalytic amount; and
b. contacting the saccharide-nucleotide and an acceptor in the presence of a transferase to form the saccharide-acceptor.

2. The process according to embodiment 1, wherein the saccharide is galactose, sialic acid, or 1-fucose.

3. A process for preparing a saccharide compound, comprising the steps of:
a. contacting a monosaccharide selected from galactose, sialic acid, and 1-fucose and a catalytic amount of a nucleotide selected from UDP, ADP, CMP, and GDP to form a saccharide-nucleotide compound.
b. contacting the saccharide-nucleotide compound and an acceptor compound in the presence of a transferase to obtain the saccharide.

4. A process for preparing a saccharide compound, comprising the steps of:
a. contacting a galactose saccharide unit and a catalytic amount of a nucleotide selected from UDP and ADP or a sialic acid or 1-fucose saccharide unit and a catalytic amount of a nucleotide selected from CMP, and GDP to form a saccharide-nucleotide compound.
b. contacting the saccharide-nucleotide compound and an acceptor compound in the presence of a transferase to obtain the saccharide.

5. A process for galactosylation of an acceptor compound, comprising the steps of:
a. contacting galactose and a nucleotide to form a galactose-nucleotide compound, wherein the nucleotide is present in a catalytic amount; and
b. contacting the galactose-nucleotide compound and an acceptor compound in the presence of a transferase to galactosylate the acceptor compound.

6. The process according to embodiment 5, wherein the galactose is obtained from sucrose in situ.

7. The process according to embodiment 5 or embodiment 6, wherein the process is driven to high conversion via the conversion of Sucrose to Glc-UDP and fructose.

8. A process for sialylation of an acceptor compound, comprising the steps of:
a. contacting sialic acid and a nucleotide to form a sialyl-nucleotide compound, wherein the nucleotide is present in a catalytic amount; and
b. contacting the sialyl-nucleotide compound and a compound with an acceptor compound in the presence of a transferase to sialylate the acceptor compound.

9. The process according to embodiment 8, wherein the sialic acid is obtained from 3′-SL or 6′-SL in situ.

10. The process according to embodiment 8 or embodiment 9, wherein the process is driven to high conversion via the conversion of lactose to galactose and glucose

11. A process for fucosylation of an acceptor compound, comprising the steps of:
a. contacting a fucose and a nucleotide to form a fucose-nucleotide compound, wherein the nucleotide is present in a catalytic amount; and
b. contacting the fucose-nucleotide compound and an acceptor compound in the presence of a transferase to fucosylate the acceptor compound.

12. The process according to embodiment 11, wherein the fucose is 1-fucose and is obtained from 2′-FL in situ.

13. The process according to embodiment 11 or embodiment 12, wherein the process is driven to high conversion via the conversion of sucrose to Glc-UDP and fructose

14. The process according to any one of embodiments 1-13, wherein the acceptor compound is a monosaccharide, a disaccharide, an oligosaccharide, or a polysaccharide.

15. The process according to any one of embodiments 1-14, wherein substantially no ATP is added to the process or generated by the process.

16. The process according to any one of embodiments 1-15, wherein the nucleotide is present in the catalytic amount is about 0.01 to about 10 molar percent relative to the saccharide amount.

17. A process for producing LNT and fructose, comprising the step of contacting LNTII, sucrose, a transferase, a sucrose synthase, an epimerase, and UDP to produce LNT and fructose, wherein galactose-UDP is produced, and wherein the UDP is present in about 0.01 molar percent to about 10 molar percent of the galactose or the sucrose.

18. The process according to embodiment 17, wherein the transferase is Cvb3GalT.

19. The process according to embodiment 18, wherein the transferase is B3GALT5

20. The process according to any one of embodiments 17-19, wherein the epimerase is GalE.

21. A process for producing LNnT and fructose, comprising contacting LNTII, sucrose, a glycosyltransferase, a sucrose synthase, an epimerase, and UDP to produce LNnT and fructose, wherein the UDP is present in about 0.01 molar percent to about 10 molar percent of the galactose or sucrose.

22. The process according to embodiment 21, wherein the glycosyltransferase is a LgtB.

23. The process according to embodiment 22, wherein the LgtB is NmLgtB.

24. The process according to embodiment 23, wherein the glycosyltransferase is B4GALT1.

25. The process according to any one of embodiments 21-24, wherein the epimerase is GalE.

26. A process for preparing LNT and fructose, comprising the steps of contacting GlcNAc, 2-chloro-1,3-dimethylimidazolinium chloride, and a base to obtain a reaction mixture comprising GlcNAc-Oxa; and contacting the reaction mixture comprising GlcNAc-Oxa, lactose, sucrose, an aminidase, a transferase, a sucrose synthase, and an epimerase, to provide LNT and fructose, wherein the UDP is present in about 0.01 molar percent to about 10% molar percent of the lactose or the sucrose.

27. The process according to embodiment 26, wherein the aminidase is Bbh1.

28. The process according to embodiment 26 or embodiment 27, wherein the transferase is Cvb3GalT.

29. The process according to any one of embodiments 26-28, wherein the epimerase is GalE.

30. The process according to any one of embodiments 26-29, wherein the base is triethylamine.

31. A process for preparing lactose and fructose, comprising contacting glucose, sucrose, a lactose synthase, a sucrose synthase, an epimerase, and UDP, to provide lactose and fructose, wherein the UDP is present in about 0.01 molar percent to about 10 molar percent of the glucose and or the sucrose.

32. The process according to embodiment 31, wherein the epimerase is GalE.

33. A process for preparing LSTa, galactose, and glucose, comprising contacting 3′-SL, LNT, CMP, a sialyltransferase, and a lactase to provide LSTa, galactose, and glucose, wherein the CMP is present in about 0.01 molar percent to about 10 molar percent of the 3′-SL.

34. The process according to embodiment 33, wherein the sialyltransferase is ST3GAL3.

35. A process for preparing LSTc, galactose, and glucose, comprising contacting 3′-SL, LNnT, CMP, a sialyltransferase, and a lactase to provide LSTc, galactose, and glucose, wherein the CMP is present in about 0.01 molar percent to about 10 molar percent of the 3′-SL.

36. The process according to embodiment 35, wherein the sialyltransferase is ST3GAL3.

37. A process for preparing LSTd, galactose, and glucose, comprising contacting 3′-SL, LNnT, CMP, a sialyltransferase, and a lactase to provide LSTd, galactose, and glucose, wherein the CMP is present in about 0.01 molar percent to about 10 molar percent of the 3′-SL.

38. The process according to embodiment 37, wherein the sialyltransferase is ST3GAL3.

39. A process for preparing DSLNT, galactose, and glucose, comprising contacting 3′-SL, LNT, CMP, a sialyltransferase, and a lactase to provide DSLNT, galactose, and glucose, wherein the CMP is present in about 0.01 molar percent to about 10% molar percent of the 3′-SL.

40. The process according to embodiment 39, wherein the sialyltransferase is ST3GAL3 and ST6GALNAC5.

41. A process for preparing LNFPI, galactose, and glucose, comprising contacting 2′-FL, LNT, GDP, Te2FT, HmFucT, and lactase to provide LNFPI, galactose, and glucose wherein the GDP is present in about 0.01 molar percent to about 10 molar percent of the 2′-FL.

42. The process according to embodiment 41, wherein the transferase is Te2FT and HmFucT.

43. A process for preparing LNFPII, galactose, and glucose, comprising contacting 2′-FL, LNT, GDP, Hp34FT, HmFucT, and lactase, wherein the GDP is present in about 0.01 molar percent to about 10 molar percent of the 2′-FL.

44. The process according to embodiment 43, wherein the transferase is Hp34FT and HmFucT.

45. A process for preparing LNFPIII, galactose, and glucose, comprising contacting 2′-FL, LNnT, GDP, FUT9, HmFucT, and lactase, to provide LNFPIII, wherein the GDP is present in about 0.01 molar percent to about 10 molar percent of the 2′-FL.

46. The process according to embodiment 45, wherein the transferase is FUT and HmFucT.

47. The process according to any one of embodiments 1-46, wherein one or more of the enzyme is immobilized.

48. The process according to any one of embodiment 47, wherein each of the enzyme is immobilized.

49. The process according to any one of embodiments 1-48, wherein the process occurs in a single reaction vessel.

50. A machine configured for the process of any one of embodiments 1-49.

51. The machine of embodiment 50, wherein the process is within a single reaction vessel.

52. LNT prepared by the process according to any one of embodiments 1-7, 14-20, and 47-49.

53. LNnT prepared by the process according to any one of embodiments 1-7, 14-16, 21-30, and 47-49.

54. Lactose prepared by a process according to any one of embodiments 1-7, 14-16, 31-32, and 47-49.

55. LSTa prepared by a process according to any one of embodiments 1-4, 7-9, 14-16, 33-34, and 47-49.

56. LSTc prepared by a process according to any one of embodiments 1-4, 7-9, 14-16, 35-36, and 47-49.

57. LSTd prepared by a process according to any one of embodiments 1-4, 7-9, 14-16, 37-38, and 47-49.

58. DSLNT prepared by a process according to any one of embodiments 1-4, 7-9, 14-16, 39-40, and 47-49.

59. LNFPI prepared by a process according to any one of embodiments 1-4, 10-15, 41-42, and 47-49.

60. LNFPII prepared by a process according to any one of embodiments 1-4, 10-16, 43-44, and 47-49.

61. LNFPIII prepared by a process according to any one of embodiments 1-7, 10-16, and 45-49.

62. A lactose derivative prepared by a process according to any one of embodiments 1-7, 14-16, and 47-49.

63. The lactose derivative according to embodiment 62 selected from Lactobionic Acid, Lactitol, Lactosucrose, Galacto-oligosaccharides, Lactulose, and a HMO. 64. A compound prepared by a process according to any one of embodiments 1-49, wherein the compound is obtained from non-animal based plant materials.

Other embodiments are as follows.

1. A method for producing a glycosylated principal product, comprising the steps of:
a. adding a catalytic amount of a sugar-nucleotide donor to a stoichiometric amount of an acceptor in the presence of a transferase to obtain a glycosylated principal product and a catalytic amount of a nucleotide; and
   regenerating the nucleotide into a regenerated sugar-nucleotide donor by contacting a catalytic amount of the nucleotide with a stoichiometric amount of a sugar donor precursor in the presence of a transferase to obtain the regenerated sugar-nucleotide donor and a secondary product.

2. The method of embodiment 1, further comprising
a. adding a catalytic amount of the regenerated sugar-nucleotide donor to the stoichiometric amount of an acceptor in the presence of a transferase to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor in the presence of a transferase to obtain the regenerated sugar-nucleotide donor and the secondary product.

3. A method for producing a glycosylated principal product comprising the step of adding a sugar donor precursor, an acceptor, a transferase, and a catalytic amount of a nucleotide to obtain a glycosylated principal product and a secondary product.

4. The method according to embodiment 3, wherein the sugar donor precursor and the nucleotide provides a sugar-nucleotide donor.

5. The method according to any one of embodiments 1-4, wherein the sugar donor precursor and the nucleotide provides a sugar-nucleotide donor precursor and wherein an auxiliary enzyme and the sugar-nucleotide donor precursor provides the sugar-nucleotide donor.

6. The method according to any one of embodiments 1-5, wherein the sugar donor precursor is a sugar comprising 2 or more sugar units.

7. The method according to embodiment 6, wherein the sugar donor precursor is a sugar comprising 2, 3, or 4 sugar units.

8. The method according to embodiment 7, wherein the sugar donor precursor is a disaccharide.

9. The method according to embodiment 7, wherein the sugar donor precursor is a trisaccharide.

10. The method according to any one of embodiments 1-9, wherein the sugar donor precursor comprises a galactose, sialic acid, fucose, or N-acetylglucosamine.

11. The method according to embodiment 10, wherein the galactose, sialic acid, fucose, or N-acetylglucosamine is in a terminal position in the sugar donor precursor.

12. The method according to any one of embodiments 1-9, wherein the sugar-nucleotide donor comprises a galactose, sialic acid, fucose, or N-acetylglucosamine

13. The method according to any one of embodiments 1-9, wherein the sugar-nucleotide donor is a galactosyl-nucleotide, sialyl-nucleotide, fucosyl-nucleotide, or N-acetylglucosaminyl-nucleotide.

14. The method according any one of embodiments 1-13, wherein the secondary product is a monosaccharide, disaccharide, or trisaccharide.

15. The method according to any one of embodiments 1-14, wherein the secondary product, in the presence of a processing enzyme, is converted to a secondary product derivative.

16. The method according to any one of embodiments 1-15, wherein the secondary product derivative, in the presence of a second processing enzyme, is converted to a modified secondary product derivative.

17. The method according any one of embodiments 1-16, wherein the acceptor is an organic compound containing a hydroxy group.

18. The method according to embodiment 17, wherein the organic compound containing a hydroxy group is a sugar.

19. The method according to any one of embodiments 1-18, wherein the acceptor is obtained from the processing enzyme converting the secondary product into the acceptor.

20. The method according to any one of embodiments 1-19, wherein the nucleotide is a uridine diphosphate, cytidine monophosphate, or guanosine diphosphate.

21. The method according to any one of embodiments 1-20, wherein the nucleotide is in an amount of 0.001 mol percent to 10 mol percent relative to the sugar donor precursor or the acceptor.

22. The method according to any one of embodiments 1-21, wherein the nucleotide is in an amount of 0.01 mol percent to 1 mol percent relative to the sugar donor precursor or the acceptor.

23. The method according to any one of embodiments 1-22, wherein the transferase is a β-1,3-galactosyl transferase, sucrase synthase, β-1,4-galactosyl transferase, sialyl transferase, fucosyl transferase, or glucosaminyl (N-acetyl) transferase 2.

24. The method according to any one of embodiments 1-23, wherein the transferase comprises a first transferase and a second transferase.

25. The method according to any one of embodiments 1-24, wherein the first transferase catalyzes a transfer of a sugar from the sugar-nucleotide donor to the acceptor to obtain the glycosylated principal product and the second transferase catalyzes a reaction of the nucleotide and the sugar donor precursor to obtain the sugar-nucleotide donor and the secondary product.

26. A method for producing a galactosylated principal product, comprising the steps of:
a. adding a catalytic amount of a sugar-nucleotide donor to a stoichiometric amount of an acceptor to obtain a galactosylated principal product and a catalytic amount of a nucleotide; and
b. regenerating the nucleotide into a regenerated sugar-nucleotide donor by contacting a catalytic amount of the nucleotide with a stoichiometric amount of a sugar donor precursor to obtain the regenerated sugar-nucleotide donor and a secondary product.

27. The method of embodiment 26, further comprising
a. adding a catalytic amount of the regenerated sugar-nucleotide donor to the stoichiometric amount of an acceptor to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor to obtain the regenerated sugar-nucleotide donor and the secondary product.

28. A method for producing a galactosylated principal product comprising the step of adding a sugar donor precursor, an acceptor, a transferase, and a catalytic amount of a nucleotide to obtain a glycosylated principal product and a secondary product.

29. The method according to any one of embodiments 26-28, wherein the acceptor is lacto-N-triose II (LNTII), glucose, or galactooligosaccharide.

30. The method according to any one of embodiments 26-29, wherein the secondary product is fructose.

31. The method according to any one of embodiments 26-30, comprising the step of obtaining the glucose by contacting the fructose in the presence of a glucose isomerase to obtain the glucose.

32. The method according to any one of embodiments 26-31, comprising the step of obtaining the LNTII by contacting lactase and N-acetylglucosamine in the presence of β-N-acetylhexosaminidase (Bbh1) to obtain the LNTII.

33. The method according to any one of embodiments 26-32, wherein the sugar donor precursor is sucrose.

34. The method according to any one of embodiments 26-33, wherein the sugar-nucleotide donor precursor is a glucose nucleotide.

35. The method according to any one of embodiments 26-34, wherein the secondary product is fructose.

36. The method according to any one of embodiments 26-35, wherein the auxiliary enzyme is a galactose epimerase.

37. The method according to any one of embodiments 26-36, wherein the nucleotide is uridine diphosphate.

38. The method according to any one of embodiments 26-36, wherein the transferase comprises a first transferase and a second transferase.

39. The method according to embodiment 38, wherein the first transferase is β-1,3-galactosyltransferase from Chromobacterium violaceum (Cvb3GalT), β-1,4-Galactosyltransferase from Neisseria meningitidis (NmLgtB), and the second transferase is sucrose synthase from Arabidopsis thaliana (AtSuSy1).

40. A method for producing a sialylated principal product, comprising the steps of:
a. adding a catalytic amount of a sugar-nucleotide donor to a stoichiometric amount of an acceptor to obtain a sialylated principal product and a catalytic amount of a nucleotide; and
b. regenerating the nucleotide into a regenerated sugar-nucleotide donor by contacting a catalytic amount of the nucleotide with a stoichiometric amount of a sugar donor precursor to obtain the regenerated sugar-nucleotide donor and a secondary product.

41. The method of embodiment 40, further comprising
a. adding a catalytic amount of the regenerated sugar-nucleotide donor to the stoichiometric amount of an acceptor to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor to obtain the regenerated sugar-nucleotide donor and the secondary product.

42. A method for producing a sialylated principal product comprising the step of adding a sugar donor precursor, an acceptor, a transferase, and a catalytic amount of a nucleotide to obtain a sialylated principal product and a secondary product.

43. The method according to any one of embodiments 40-42, wherein the sugar donor precursor is 3′-sialyllactose.

44. The method according to any one of embodiments 40-43, wherein the sugar-nucleotide donor is a N-acetyl neuraminic acid nucleotide.

45. The method according to any one of embodiments 40-44, wherein the secondary product is lactose.

46. The method according to any one of embodiments 40-45, wherein the secondary product, in the presence of a processing enzyme, is converted to a secondary product derivative.

47. The method according to embodiment 46, wherein the processing enzyme is lactase.

48. The method according to embodiment 47, wherein the secondary product derivative is glucose and galactose.

49. The method according to any one of embodiments 40-48, wherein the nucleotide is cytidine monophosphate.

50. The method according to any one of embodiments 40-49, wherein the transferase comprises a first transferase and a second transferase.

51. The method according to embodiment 50, wherein the first transferase is beta-galactoside alpha-2,6-sialyltransferase 1 (ST6GAL1), CMP-N-acetylneuraminate-beta-1,4-galactoside alpha-2,3-sialyltransferase (ST3GAL3) or Alpha-N-acetylgalactosaminide alpha-2,6-sialyltransferase 5 (ST6GALNAC5) and second transferase is CMP-N-acetylneuraminate-beta-galactosamide-alpha-2,3-sialyltransferase 4 (ST3GAL4).

52. The method according to embodiments 50, wherein the first transferase ST3GAL3 and ST6GALNAC5.

53. A method for producing a fucosylated principal product, comprising the steps of:
a. adding a catalytic amount of a sugar-nucleotide donor to a stoichiometric amount of an acceptor to obtain a fucosylated principal product and a catalytic amount of a nucleotide; and
b. regenerating the nucleotide into a regenerated sugar-nucleotide donor by contacting a catalytic amount of the nucleotide with a stoichiometric amount of a sugar donor precursor to obtain the regenerated sugar-nucleotide donor and a secondary product.

54. The method of embodiment 53, further comprising
a. adding a catalytic amount of the regenerated sugar-nucleotide donor to the stoichiometric amount of an acceptor to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor to obtain the regenerated sugar-nucleotide donor and the secondary product.

55. A method for producing a fucosylated principal product comprising the step of adding a sugar donor precursor, an acceptor, a transferase, and a catalytic amount of a nucleotide to obtain a fucosylated principal product and a secondary product.

56. The method according to any one of embodiments 53-55, wherein the sugar donor precursor is a 2′-fucosyllactose.

57. The method according to any one of embodiments 53-56, wherein the sugar-nucleotide donor is a fucose nucleotide.

58. The method according to any one of embodiments 53-57, wherein the secondary product is lactose.

59. The method according to any one of embodiments 53-58, wherein the secondary product, in the presence of a processing enzyme, is converted to a secondary product derivative.

60. The method according to embodiment 59, wherein the processing enzyme is lactase.

61. The method according to embodiment 59 or embodiment 60, wherein the secondary product derivative is glucose and galactose.

62. The method according to any one of embodiments 59-61, wherein the secondary product derivative, in the presence of a second processing enzyme, is converted to a modified secondary product derivative.

63. The method according to claim 62, wherein the second processing enzyme is D-Galactose isomerase source from Geobacillus stearothermophilus (GsAI) and the modified secondary product derivative is tagatose.

64. The method according to any one of embodiments 53-63, wherein the nucleotide is guanosine diphosphate.

65. The method according to any one of embodiments 53-64, wherein the transferase comprises a first transferase and a second transferase.

66. The method according to embodiment 65, wherein the first transferase is α-1,2-fucosyltransferase from Thermosynechococcus vestitus (Te2FT), fucosyl transferase 9 (FUT9) or fucosyl transferase 3 (FUT3) and second transferase is α-1,2-fucosyltransferase from Helicobacter mustelae (HmFucT) or fucosyl transferase (FUT1).

67. A method for producing a N-acetylglucosaminylated principal product, comprising the steps of:
a. adding a catalytic amount of a sugar-nucleotide donor to a stoichiometric amount of an acceptor to obtain a N-acetylglucosaminylated principal product and a catalytic amount of a nucleotide; and
b. regenerating the nucleotide into a regenerated sugar-nucleotide donor by contacting a catalytic amount of the nucleotide with a stoichiometric amount of a sugar donor precursor to obtain the regenerated sugar-nucleotide donor and a secondary product.

The method of embodiment 1, further comprising
a. adding a catalytic amount of the regenerated sugar-nucleotide donor to the stoichiometric amount of an acceptor to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor to obtain the regenerated sugar-nucleotide donor and the secondary product.

The method of embodiment 1, further comprising
a. adding a catalytic amount of the regenerated sugar-nucleotide donor to the stoichiometric amount of an acceptor to obtain the glycosylated principal product and the catalytic amount of a nucleotide; and
b. regenerating the nucleotide into the regenerated sugar-nucleotide donor by contacting the catalytic amount of a nucleotide with the stoichiometric amount of a sugar donor precursor to obtain the regenerated sugar-nucleotide donor and the secondary product.

69. A method for producing a N-acetylglucosaminylated principal product comprising the step of adding a sugar donor precursor, an acceptor, a transferase, and a catalytic amount of a nucleotide to obtain a N-acetylglucosaminylated principal product and a secondary product.

70. The method according to any one of embodiments 67-69, wherein the sugar donor precursor is lacto-N-biose.

71. The method according to any one of embodiments 67-70, wherein the sugar-nucleotide donor is a N-acetylglucosamine nucleotide.

72. The method according to any one of embodiments 67-71, wherein the secondary product is galactose.

73. The method according to claim any one of embodiments 67-72, wherein the secondary product derivative is tagatose.

74. The method according to any one of embodiments 61-73, wherein the processing enzyme D-galactose isomerase sourced from Geobacillus stearothermophilus (GsAI).

75. The method according to any one of embodiments 61-74, wherein the nucleotide is uridine diphophate.

76. The method according to any one of embodiments 61-75, wherein the transferase is β-1,3-N-Acetyl-Hexosaminyl-transferase from Neisseria meningitidis (NmLgtA).

77. The method according to any one of embodiments 61-76, wherein the transferase comprises a first transferase and a second transferase.

78. The method according to embodiment 77, wherein the first transferase is glucosaminyl (N-acetyl) transferase 2 (GCNT2) and second transferase is β-1,3-N-Acetyl-Hexosaminyl-transferase from Neisseria meningitidis (NmLgtA).

79. The method according to any one of embodiments 1-78, wherein glucose is D-glucose

80. The method according to any one of embodiments 1-79, wherein galactose is D-galactose.

81. The method according to any one of embodiments 1-80, wherein fucose is L-fucose.

82. A glycosylated compound prepared according to any one of embodiments 1-81.

83. A galactosylated compound prepared according to any one of embodiments 1-39.

84. A sialylated compound prepared according to any one of embodiments 1-25, 40-52, or 79-81.

85. A fucosylated compound prepared according to any one of embodiments 1-25, 53-66, or 79-81.

86. A N-acetylglucosaminylated compound prepared according to any one of embodiments 1-25, 67-78, or 79-81.

87. A lactose derivative prepared by a process according to any one of embodiments 1-81.

88. The lactose derivative according to claim 87 selected from lactobionic acid, lactitol, lactosucrose, galacto-oligosaccharides, lactulose, and an HMO.

89. A compound prepared by a method according to any one of embodiments 1-81, wherein the compound is obtained from non-animal based plant materials.

90. A machine configured for the method of any one of embodiments 1-81

91. The machine according to embodiments 90, wherein the process is within a single reaction vessel.

In order that the invention described herein may be more fully understood, the following examples are set forth. It should be understood that these examples are for illustrative purposes only and are not to be construed as limiting this invention in any manner.

### EXAMPLES

Abbreviations
3′-SL: 3′Sialyllactose
2′-FL: 2′-Fucosyllactose
LNFPI: Lacto-N-fucopentaose I
LNFPII: Lacto-N-fucopentaose II
LNFPIII: Lacto-N-fucopentaose III
LNT: Lacto-N-tetraose
LNnT: Lacto-N-neotetraose
LNTII: Lacto-N-triose II
DSLNT: Disialyllacto-N-tetraose
UDP: Uridine 5′-diphosphate
UTP: Uridine 5′-triphosphate
ADP: Adenosine 5′-diphosphate
ATP: Adenosine 5′-triphosphate
CMP: Cytidine 5′-monosphate
CDP: Cytidine 5′-diphosphate
CTP: Cytidine 5′-triphosphate
GDP: Guanosine 5′-diphosphate
GTP: Guanosine 5′-triphosphate
TDP: Thymidine 5′-diphosphate
Tris: Tris(hydroxymethyl)aminomethane
DMC: 2-Chloro-1,3-dimethylimidazolinium chloride
Bbh1: β-N-acetylglucosaminidase
GOS: Galactooligosaccharide
DFL: Difucosyllactose
3-FL: 3-Fucosyllactose
LSTa: Sialyllacto-N-tetraose a LS-Tetrasaccharide a
LSTb: Sialyllacto-N-tetraose b/LS-Tetrasaccharide b
LSTc: Sialyllacto-N-neotetraose c/LS-Tetrasaccharide c
LSTd: Sialyllacto-N-neotetraose d/LS-Tetrasaccharide d
Cvb3GalT: β-1,3-Galactosyl transferase from Chromobacterium violaceum
AtSuSy1: Sucrose synthase from Arabidopsis thaliana
Beta-galactoside alpha-2,6-sialyltransferase 1 (ST6GAL1)
ST3GAL3: CMP-N-acetylneuraminate-beta-1,4-galactoside alpha-2,3-sialyltransferase 3
ST3GAL4: CMP-N-acetylneuraminate-beta-galactosamide-alpha-2,3-sialyltransferase 4
ST6GALNAC5: alpha-N-acetylgalactosaminide alpha-2,6-sialyltransferase 5
ST3GAL4: CMP-N-acetylneuraminate-beta-galactosamide-alpha-2,3-sialyltransferase 4
Te2FT: α-1,2-Fucosyltransferase from Thermosynechococcus vestitus
Hp34FT: α1-3/4-Fucosyltransferase from Helicobacter pylori
FUT9: Fucosyl transferase 9
FUT3: Fucosyl transferase 3
HmFucT: α-1,2-Fucosyltransferase from Helicobacter mustelae (HmFucT)
FUT1: Fucosyl transferase
Pyr: Pyruvate
AcPi: Acetyl phosphate
PPi: Inorganic pyrophosphate
Pi: Inorganic phosphate
NADP+: Nicotinamide adenine dinucleotide phosphate oxidized form
NADPH: Nicotinamide adenine dinucleotide phosphate reduced form
PolyPₙ and PolyPₙ₋₁: Polyphosphate; n indicates number of phosphate units
HK: Hexokinase
FDH: Formate dehydrogenase
PtxD: Phosphite Oxidoreductase
GOS: Galactooligosaccharide
Lactose synthase E.C. 2.4.1.22: Generates lactose from glucose and UDP-galactose. It consists of a 1:1 complex of galactosyltransferase (B4GALT1 or B4GALT2) and alpha-lactalbumin.
UDP-sugar pyrophosphorylase (USP) E.C. 2.7.7.64: Catalyzes a reversible transfer of the uridyl group from UTP to sugar-1-phosphate, producing UDP-sugar and pyrophosphate (PPi)
UTP-Glucose 1-phosphate uridylyltransferase (GalU) E.C. 2.7.7.9: Catalyzes the conversion of glucose-1-phosphate or galactose-1-phosphate to UDP-glucose or UDP-galactose, respectively.
Galactokinase (GalK) E.C. 2.7.1.6: Catalyzes the phosphorylation of galactose to galactose-1-phosphate (Gal-1P) via consumption of one ATP unit.
Inorganic pyrophosphatase (Ppa) E.C. 3.6.1.1: Catalyzes the hydrolysis of pyrophosphate (PPi) to two monophosphate ions (Pi).
Alkaline Phosphatases E.C. 3.1.3.1: Catalyzes the hydrolysis of a nucleotide (NMP, NDP, NTP) into the corresponding nucleoside (N) and a phosphate.
CIAP: Calf Intestinal Alkaline Phosphatase

### Example 1: Cell Free Production of Simple Glycan

1a. Reagents and Materials:

The following chemicals reagents are used to synthesize the carbohydrates, glycans or human milk oligosaccarides: Sucrose (Carbosynth: OS02339), Glucose (α-D(+)-Glucose, 99+%, anhydrous, Thermo Scientific: AC170080010), CMP (Cytidine 5′-monophosphate disodium salt; Carbosynth: NC05637), UDP (Uridine 5′-diphosphate disodium salt; Carbosynth: NU03399), GDP (Guanosine 5′-diphosphate disodium salt; Carbosynth: NG09782), 2′-Fucosyllactose (2′-FL; Carbosynth: OF06739), 3′-Sialyllactose (3′-SL; Carbosynth: OS04397), Lactose (D-Lactose monohydrate; Fisher Scientific: L5-500), GlcNAc (N-Acetyl-D-glucosamine; Sigma Aldrich: A8625), Tris (TRIS, 1.0M buffer solution., pH 7.5; Alfa Aesar: J62993AP), HEPES (Thermo Scientific: AAA1477730), PIPES (Thermo Scientific: AC172615000) and MgCl₂ (Magnesium Chloride; Macron Fine Chemicals: 595804). All water is obtained from a BarnStead Nanopure water purifier (Thermo Scientific, 18.5 MOhm-cm). LNTII, LNT and LNnT are produced in-house from lactose.

Mammalian enzymes ST3GAL3, ST3GAL4, ST6GAL1, B4GALT1, B3GALT5, B3GNT2, FUT9 and ST6GalNAc5 are purchased from Glyco Expression Technologies Inc. (Athens, Georgia). Lactase is purchased from Sunson Enzymes, and human a-lactalbumin is purchased from Athens Research & Technology. SuSy (Sucrose synthase; source: Glycine Max), GalE (UDP-glucose 4-epimerase; source: Bifidobacterium longum), NmLgtB (β-1,4-Galactosyltransferase; source: Neisseria meningitidis), Cvβ3GalT (β-1,3-galactosyl transferase; source: Chromobacterium violaceum), Te2FT (α-1,2-fucosyltransferase; source: Thermosynechococcus vestitus), HmFucT (α-1,2-fucosyltransferase; source: Helicobacter mustelae), and Hp34FT (α1-3/4-fucosyltransferase; source: Helicobacter pylori) are produced in-house recombinantly in E. coli (BL21/DE3). The enzymes are purified from the soluble lysate by affinity chromatography (NiNTA) and the buffer is exchanged by dialyzing against 50 mM Tris pH 7.5. The enzymes are supplemented with 10% (w/w) glycerol and frozen at −80° C. for storage. The plasmids used for protein expression are produced by Genewiz or Genscript by custom synthesis of the insert and splicing into a commercial pET28a vector (Novagen).

### Example 1b: LSTa and LNT

1b. Reagents and materials: The following chemicals reagents were used to synthesize Lactose, LSTa and LNT: Sucrose (Carbosynth: OS02339), Glucose (α-D(+)-Glucose, 99+%, anhydrous, Thermo Scientific: AC170080010), CMP (Cytidine disodium salt; Carbosynth: NC05637), UDP (Uridine 5′-diphosphate disodium salt; Carbosynth: NU03399), 3′-Sialyllactose (3′-SL; Carbosynth: OS04397), Lactose (D-Lactose monohydrate; Fisher Scientific: L5-500), Tris (TRIS, 1.0M buffer solution, pH 7.5; Alfa Aesar: J62993AP), HEPES (Thermo Scientific: AAA1477730), PIPES (Thermo Scientific: AC172615000) and MgCl₂ (Magnesium Chloride; Macron Fine Chemicals: 595804). All water was obtained from a BarnStead Nanopure water purifier (Thermo Scientific, 18.5 MOhm-cm). LNTII and LNT were produced in-house from lactose.

Mammalian enzymes ST3GAL3, ST3GAL4, ST6GAL1, B4GALT1, B3GALT5, B3GNT2, FUT9 and ST6GalNAc5 were purchased from Glyco Expression Technologies Inc. (Athens, Georgia). Lactase was purchased from Sunson Enzymes, and human a-lactalbumin was purchased from Athens Research & Technology. SuSy (Sucrose synthase; source: Glycine max), GalE (UDP-glucose 4-epimerase; source: Bifidobacterium longum), NmLgtB (β-1,4-Galactosyltransferase; source: Neisseria meningitidis), Cvβ3GalT (β-1,3-galactosyl transferase; source: Chromobacterium violaceum), Te2FT (α-1,2-fucosyltransferase; source: Thermosynechococcus vestitus), HmFucT (α-1,2-fucosyltransferase; source: Helicobacter mustelae), and Hp34FT (α1-3/4-fucosyltransferase; source: Helicobacter pylori) were produced in-house recombinantly in E. coli (BL21/DE3). The enzymes were purified from the soluble lysate by affinity chromatography (NiNTA) and the buffer was exchanged by dialyzing against 50 mM Tris pH 7.5. The enzymes were supplemented with 10% (w/w) glycerol and frozen at −80° C. for storage. The plasmids used for protein expression were produced by Genewiz or Genscript by custom synthesis of the insert and splicing into a commercial pET28a vector (Novagen).

### Example 2a: Production of LNT, LNnT, and Lactose Via Galactosylation

The following examples illustrate the step-by-step syntheses of LNT, LNnT, lactose, GOS, LNFPI, LNFPII, LNFPIII, LSTa, LSTc, LSTd and DSLNT as shown in FIGS. 1-3. The target glycans may be synthesized step-by-step by sequentially combining below examples. Alternatively, the target glycans may be synthesized from any advanced glycan building block.

A. Synthesis of LNT from LNTII

LNT is produced from LNTII, sucrose, and catalytic amounts of UDP using the three enzymes Cvb3GalT, SuSy and GalE (FIG. 1B). The reaction mixture is composed of 25 mM LNTII, 50 mM sucrose, 5 mM UDP, 400 μg/ml SuSy, 800 μg/ml Cvb3GalT, 400 μg/m1GalE and 10 mM MgCl₂ in 50 mM Tris pH 7.0 at 37° C.

B. Synthesis of LNnT from LNTII

LNnT is produced from LNTII, sucrose, and catalytic amounts of UDP using the three enzymes NmLgtB or B4GALT1, SuSy and GalE (FIG. 1C). The reaction mixture is composed of 25 mM LNTII, 50 mM sucrose, 0.1-10 mM UDP, 400 μg/ml SuSy, 800 μg/ml Cvb3GalT, 400 μg/ml GalE and 10 mM MgCl₂ in 20-100 mM buffer in the pH range of 6.5 to 7.5 at 37° C.

C. Synthesis of Lactose from Glucose

Lactose is produced from Glucose, sucrose, and catalytic amounts of UDP using the four enzymes B4GALT1, α-lactalbumin, SuSy and GalE (FIG. 1E). The reaction mixture is composed of 25 mM Glucose, 100 mM sucrose, 0.1-10 mM UDP, 100 μg/ml B4GALT1, 100 μg/ml α-lactalbumin, 400 μg/ml SuSy, 400 μg/ml GalE and 10 mM MgCl₂ in 20-100 mM buffer in the pH range of 6.5 to 7.5 at 37° C.

D. Synthesis of Galacto-oligosaccharide (GOS) from Sucrose

GOS is produced from sucrose and catalytic amounts of UDP using the three enzymes β-1,4-galactosyltransferase, SuSy and GalE (FIG. 1F). The reaction mixture is composed of 2 M sucrose, 0.1-10 mM UDP, 400 μg/ml SuSy, 800 μg/ml β-1,4-galactosyltransferase, 400 μg/ml GalE and 10 mM MgCl₂ in 20-100 mM buffer in the pH range of 6.5 to 7.5 at 37° C.

### Example 2b. Production of LSTa, LSTc, LSTd, and DSLNT. Sialylation

A. Synthesis of LSTa from LNT

LSTa is produced from LNT, 3′-SL, and catalytic amounts of CMP using the three enzymes ST3GAL4, ST3GAL3 and Lactase (FIG. 2A). The reaction mixture is composed of 25 mM LNT, 30 mM 3′-SL, 0.1-10 mM CMP, 100 μg/ml ST3GAL4, 150 μg/ml ST3GAL3, 400 μg/ml lactase and 10 mM MgCl₂ in 20-100 mM buffer in the pH range of 6.5 to 7.5 at 37° C.

B. Synthesis of LSTc from LNnT

LSTc is produced from LNnT, 3′-SL, and catalytic amounts of CMP using the three enzymes ST3GAL4, ST6GAL1 and Lactase (FIG. 2B). The reaction mixture is composed of 25 mM LNnT, 30 mM 3′-SL, 0.1-10 mM CMP, 400 μg/ml ST3GAL4, 400 μg/ml ST6GAL1, 400 μg/ml lactase and 10 mM MgCl₂ in 20-100 mM buffer in the pH range of 6.5 to 7.5 at 37° C.

C. Synthesis of LSTd from LNnT

LSTd is produced from LNnT, 3′-SL, and catalytic amounts of CMP using the three enzymes ST3GAL4, ST3GAL3 and Lactase (FIG. 2C). The reaction mixture is composed of 25 mM LNnT, 30 mM 3′-SL, 0.1-10 mM CMP, 400 μg/ml ST3GAL4, 400 μg/ml ST3GAL3, 400 μg/ml lactase and 10 mM MgCl₂ in 20-100 mM buffer in the pH range of 6.5 to 7.5 at 37° C.

D. Synthesis of DSLNT from LNT

DSLNT is produced from LNT, 3′-SL, and catalytic amounts of CMP using the four enzymes ST3GAL4, ST3GAL3, ST6GALNAC5 and Lactase (FIG. 2D). The reaction mixture is composed of 25 mM LNT, 60 mM 3′-SL, 0.1-10 mM CMP, 400 μg/ml ST3GAL4, 400 μg/ml ST3GAL3, 400 μg/ml ST6GALNAC5, 400 μg/ml lactase and 10 mM MgCl₂ in 20-100 mM buffer in the pH range of 6.5 to 7.5 at 37° C.

### Example 2c. Production of LNFPI, II, III Etc. Via Fucosylation

A. Synthesis of LNFPI from LNT

LNFPI is produced from LNT, 2′-FL, and catalytic amounts of CMP using the three enzymes HmFucT, Te2FT and Lactase (FIG. 3A). The reaction mixture is composed of 25 mM LNT, 30 mM 2′-FL, 0.1-10 mM GDP, 400 μg/ml HmFucT, 400 μg/ml Te2FT, 400 μg/ml lactase and 10 mM MgCl₂ in 20-100 mM buffer in the pH range of 6.5 to 7.5 at 37° C.

B. Synthesis of LNFPII from LNT

LNFPII is produced from LNT, 2′-FL, and catalytic amounts of CMP using the three enzymes HmFucT, Hp34FT or FUT5, and Lactase (FIG. 3B). The reaction mixture is composed of 25 mM LNT, 30 mM 2′-FL, 0.1-10 mM GDP, 400 μg/ml HmFucT, 400 μg/ml Hp34FT or FUT5, 400 μg/ml lactase and 10 mM MgCl₂ in 20-100 mM buffer in the pH range of 6.5 to 7.5 at 37° C.

C. Synthesis of LNFPIII from LNnT

LNFPIII is produced from LNnT, 2′-FL, and catalytic amounts of CMP using the three enzymes HmFucT, FUT9 and Lactase (FIG. 3C). The reaction mixture is composed of 25 mM LNnT, 30 mM 2′-FL, 0.1-10 mM GDP, 400 μg/ml HmFucT, 400 μg/ml FUT9, 400 μg/ml lactase and 10 mM MgCl₂ in 20-100 mM buffer in the pH range of 6.5 to 7.5 at 37° C.

### Example 3a. Production of LNT and Lactose Via Galactosylation

A. Synthesis of LNT from LNTII

LNT was produced from LNTII, sucrose, and catalytic amounts of UDP using the three enzymes Cvb3GalT, SuSy and GalE (FIG. 1B). The reaction mixture was composed of 25 mM LNTII, 50 mM sucrose, 5 mM UDP, 400 μg/ml SuSy, 800 μg/ml Cvb3GalT, 400 μg/ml GalE and 10 mM MgCl₂ in 50 mM Tris pH 7.0 at 37° C. Conversion from LNTII to LNT was monitored by HPLC (Thermo Vanquish, Trinity P1 column, charged aerosol detector). Conversions of 10.6%, 37.4% and 77.7% were observed at 0.5 hr, 1 hr and 17 hrs, respectively.

B. Synthesis of Lactose from Glucose

Lactose was produced from Glucose, sucrose, and catalytic amounts of UDP using the four enzymes B4GALT1, α-lactalbumin, SuSy and GalE (FIG. 1E) in a four-steps process. In step one, the lactose synthase complex was assembled in the mixture composed of 25 mM Glucose, 200 μg/ml B4GALT1, 80 μg/ml α-lactalbumin, 10 mM MnCl2, 1 mM CaCl2, and 50 mM HEPES pH 6.8, incubated at 4° C. for 1 hour. In step two, UDP-galactose was synthesized in a s reaction mixture composed of 100 μg/ml SuSy, 100 μg/ml GalE, 6 mM UDP, 50 mM Sucrose and 10 mM MnCl2 in 50 mM HEPES pH 6.8, incubated for 1 hour at 37° C. In step three, equal volumes of the two mixtures from step one and step two were combined and incubated at 4° C. for 30 minutes. In this, the UDP-galactose produced in step two was used to stabilize the lactose synthase complex. In step four, the reaction mixture was transferred to 37° C. to facilitate the conversion of glucose to lactose. Conversion from Glucose to lactose was monitored by HPLC (Thermo Vanquish, Trinity P1 column, charged aerosol detector). Conversions of 8.9%, 14.3%, 20.8% and 25.4% were observed at 0.25 hr, 0.5 hr, 1 hr and 17 hrs, respectively.

### Example 3b. Production of LSTa Via Sialylation of LNT

Synthesis of LSTa from LNT

LSTa was produced from LNT, 3′-SL, and catalytic amounts of CMP using

the three enzymes ST3GAL4, ST3GAL3 and Lactase (FIG. 2A). The reaction mixture was composed of 25 mM LNT, 30 mM 3′-SL, 5 mM CMP, 100 μg/ml ST3GAL4, 150 μg/ml ST3GAL3, 400 μg/ml lactase and 10 mM MgCl₂ in 50 mM Tris pH 7.0 at 37° C. Conversion from LNT to LSTa was monitored by HPLC (Thermo Vanquish, Trinity P1 column, charged aerosol detector). A conversion of approximately 5% was observed after 21 hours.

### Example 4. Cell Free Production of Simple Glycans

4a. Reagents and Materials

The following chemical reagents are used to synthesize the carbohydrates,

glycans or human milk oligosaccarides: Sucrose (Carbosynth: OS02339), Glucose (α-D(+)-Glucose, 99+%, anhydrous, Thermo Scientific: AC170080010), CMP (Cytidine disodium salt; Carbosynth: NC05637), UDP (Uridine 5′-diphosphate disodium salt; Carbosynth: NU03399), GDP (Guanosine 5′-diphosphate disodium salt; Carbosynth: NG09782), 2′-Fucosyllactose (2′-FL; Carbosynth: OF06739), 3′-Sialyllactose (3′-SL; Carbosynth: OS04397), Lactose (D-Lactose monohydrate; Fisher Scientific: L5-500), Lacto-N-biose (LNB; Chemily GlycoScience: OS03002); GlcNAc (N-Acetyl-D-glucosamine; Sigma Aldrich: A8625), Tris (TRIS, 1.0M buffer solution., pH 7.5; Alfa Aesar: J62993AP), HEPES (Thermo Scientific: AAA1477730), PIPES (Thermo Scientific: AC172615000) and MgCl₂ (Magnesium Chloride; Macron Fine Chemicals: 595804). All water is obtained from a BarnStead Nanopure water purifier (Thermo Scientific, 18.5 MOhm-cm). LNTII, LNT and LNnT are produced in-house from lactose.

Mammalian enzymes ST3GAL3, ST3GAL4, ST6GAL1, B4GALT1, B3GALT5, GCNT2, B3GNT2, FUT1, FUT3, FUT9 and ST6GalNAc5 are purchased from Glyco Expression Technologies Inc. (Athens, Georgia). Lactase is purchased from Sunson Enzymes, and human a-lactalbumin is purchased from Athens Research & Technology. SuSy (Sucrose synthase; source: Glycine max), GalE (UDP-glucose 4-epimerase; source: Bifidobacterium longum), NmLgtB (β-1,4-Galactosyltransferase; source: Neisseria meningitidis), NmLgtA (β-1,3-N-Acetyl-Hexosaminyl-transferase; source: Neisseria meningitidis), Cvβ3GalT (β-1,3-galactosyl transferase; source: Chromobacterium violaceum), Te2FT (α-1,2-fucosyltransferase; source: Thermosynechococcus vestitus), HmFucT (α-1,2-fucosyltransferase; source: Helicobacter mustelae), GsAI (D-Galactose isomerase; source: Geobacillus stearothermophilus) and Hp34FT (α1-3/4-fucosyltransferase; source: Helicobacter pylori) are produced in-house recombinantly in E. coli (BL21/DE3). The enzymes are purified from the soluble lysate by affinity chromatography (NiNTA) and the buffer is exchanged by dialyzing against 50 mM Tris pH 7.5. The enzymes are supplemented with 10% (w/w) glycerol and frozen at −80° C. for storage. The plasmids used for protein expression are produced by Genewiz or Genscript by custom synthesis of the insert and splicing into a commercial pET28a vector (Novagen).

4b. Reagents and Materials:

The following chemicals reagents were used to synthesize Lactose, LNT and LNnT: Sucrose (Carbosynth: OS02339), Glucose (α-D(+)-Glucose, 99+%, anhydrous, Thermo Scientific: AC170080010), UDP (Uridine 5′-diphosphate disodium salt; Carbosynth: NU03399), Lactose (D-Lactose monohydrate; Fisher Scientific: L5-500), Sodium Phosphate Dibasic Heptahydrate (Fisher Chemical: S373-500) and MgCl₂ (Magnesium Chloride; Macron Fine Chemicals: 595804). All water was obtained from a BarnStead Nanopure water purifier (Thermo Scientific, 18.5 MOhm-cm). LNTII was produced in-house from lactose.

AtSuSy1 (Sucrose synthase; source: Arabidopsis thaliana), GalE (UDP-glucose 4-epimerase; source: Bifidobacterium longum), NmLgtB (β-1,4-Galactosyltransferase; source: Neisseria meningitidis) and Cvβ3GalT galactosyl transferase; source: Chromobacterium violaceum) were produced in-house recombinantly in E. coli (BL21/DE3). The enzymes were purified from the soluble lysate by affinity chromatography (NiNTA) and the buffer was exchanged by dialyzing against 50 mM Tris pH 7.5. The enzymes were supplemented with 10% (w/w) glycerol and frozen at −80° C. for storage. The plasmids used for protein expression were produced by Genewiz or Genscript by custom synthesis of the insert and splicing into a commercial pET28a vector (Novagen).

### Example 5a. Fucosylation to Produce LNFPI, LNFPII, LNFPIII, DFL, and 3-FL

A. Synthesis of LNFPI from LNT

LNFPI is produced from LNT, 2′-FL, and catalytic amounts of GDP using the four enzymes HmFucT, Te2FT, Lactase and GsAI (FIG. 11A). The reaction mixture is composed of 25 mM LNT, 30 mM 2′-FL, 0.1-10 mM GDP, 400 μg/ml HmFucT, 400 μg/ml Te2FT, 400 μg/ml lactase and 10 mM MgCl₂ in 20-100 mM buffer in the pH range of 4.5 to 7.5 at 37° C.

B. Synthesis of LNFPII from LNT

LNFPII is produced from LNT, 2′-FL, and catalytic amounts of GDP using the four enzymes HmFucT, Hp34FT, Lactase and GsAI (FIG. 11B). The reaction mixture is composed of 25 mM LNT, 30 mM 2′-FL, 0.1-10 mM GDP, 400 μg/ml HmFucT, 400 μg/ml Hp34FT or FUT5, 400 μg/ml lactase and 10 mM MgCl₂ in 20-100 mM buffer in the pH range of 4.5 to 7.5 at 37° C.

C. Synthesis of LNFPIII from LNnT

LNFPIII is produced from LNnT, 2′-FL, and catalytic amounts of GDP using the four enzymes HmFucT, FUT9, Lactase and GsAI (FIG. 11C). The reaction mixture is composed of 25 mM LNnT, 30 mM 2′-FL, 0.1-10 mM GDP, 400 μg/ml HmFucT, 400 μg/ml FUT9, 400 μg/ml lactase and 10 mM MgCl₂ in 20-100 mM buffer in the pH range of 4.5 to 7.5 at 37° C.

D. Synthesis of DFL from 2′-FL

HP34FT, Lactase and GsAI (FIG. 11D). The reaction mixture is composed of mM 2′-FL, 0.1-10 mM GDP, 400 μg/ml HmFucT, 400 μg/ml Hp34FT, 400 μg/ml lactase, 400 μg/ml GsAI and 10 mM MgCl₂ in 20-100 mM buffer in the pH range of 4.5 to 7.5 at 37° C.

E. Synthesis of 3-FL from 2′-FL

3-FL is produced from 2′-FL and catalytic amounts of GDP using the five enzymes HmFucT, HP34FT, Lactase, GsAI and Fucosidase (FIG. 11E). The reaction mixture is composed of 50 mM 2′-FL, 0.1-10 mM GDP, 400 μg/ml HmFucT, 400 μg/ml Hp34FT, 400 μg/ml lactase, 400 μg/ml GsAI, 400 μg/ml Fucosidase and 10 mM MgCl₂ in 20-100 mM buffer in the pH range of 4.5 to 7.5 at 37° C.

### Example 5b. N-Acetylglucosaminylation with Lacto-N-biose

A. Synthesis of LNTII from Lacto-N-biose (LNB).

LNTII is produced from LNB, lactose, and catalytic amounts of UDP using the two enzymes NmLgtA, and GsAI (FIG. 12A). The reaction mixture is composed of 25 mM LNB, 30 mM lactose, 0.1-10 mM UDP, 400 μg/ml NmLgtA and 400 μg/ml GsAI and 10 mM MgCl₂ in 20-100 mM buffer in the pH range of 4.5 to 7.5 at 37° C.

B. Synthesis of β-1,6-GlcNAc-LNnT from LNnT.

β-1,6-GlcNAc-LNnT is produced from LNnT, LNB, and catalytic amounts of UDP using the three enzymes NmLgtA (or B3GNT2), GCNT2, and GsAI (FIG. 12B). The reaction mixture is composed of 25 mM LNnT, 30 mM LNB, 0.1-10 mM UDP, 400 μg/ml NmLgtA, 400 μg/ml GCNT2 and 400 μg/ml GsAI and 10 mM MgCl₂ in 20-100 mM buffer in the pH range of 4.5 to 7.5 at 37° C.

### Example 6

A. Synthesis of LNT from LNTII

LNT was produced from LNTII, sucrose, and catalytic amounts of UDP using the three enzymes Cvb3GalT, AtSuSy1, and GalE (FIG. 9B). The reaction mixture was composed of 250 mM LNTII, 1.0 M sucrose, 1 mM UDP, 250 μg/ml AtSuSy1, 500 μg/ml Cvb3GalT, 500 μg/ml GalE and 10 mM MgCl₂ in 50 mM phosphate buffer pH 6.6 at 37° C. Conversion from LNTII to LNT was monitored by HPLC (Agilent 1100, amino column, evaporative light scattering detector). Conversion of 86% was observed after 16 hrs., 1 hr and 17 hrs.

B. Synthesis of LNnT from LNTII

LNnT was produced from LNTII, sucrose, and catalytic amounts of UDP using the three enzymes NmLgtB, AtSuSy1 and GalE (FIG. 9C). The reaction mixture was composed of 500 mM LNTII, 1.0 M sucrose, 5 mM UDP, 400 μg/ml AtSuSy1, 200 μg/ml NmLgtB, 400 μg/ml GalE and 10 mM MgCl₂ in 250 mM phosphate pH 6.0 at 37° C. Conversion from LNTII to LNT was monitored by HPLC (Agilent 1100, amino column, evaporative light scattering detector). Conversions of 36%, 64%, 77% and 96% were observed at 2 hrs, 4 hrs, 6 hrs. and 24 hrs., respectively resulting in the production of 500 mg of LNnT product.

C. Synthesis of Lactose from Glucose

Lactose was produced from Glucose, sucrose, and catalytic amounts of UDP using the three enzymes NmLgtB, AtSuSy1 and GalE (FIG. 9E). The reaction mixture was composed of 500 mM Glucose, 1.0 M sucrose, 5 mM UDP, 400 μg/ml NmLgtB, 400 μg/ml AtSuSy1, 400 μg/ml GalE and 10 mM MgCl₂ in 250 mM phosphate pH 6.0 at 37° C. Conversion from Glucose to lactose was monitored by TLC using a p-anisaldehyde stain. An approximate conversion of 50% was observed after 24 hrs. resulting in the production of 2.5 g of lactose product.

### Example 7: Synthesis of Lactose from Glucose

Lactose is produced from Glucose, sucrose, and catalytic amounts of UDP using the four enzymes NmLgtB, AtSuSy1, GalE and Glucose (xylose) isomerase (EC 5.3. 1.5, D-xylose aldose-ketose-isomerase) (FIG. 9F). The reaction mixture is composed of 500 mM Glucose, 1.0 M sucrose, 5 mM UDP, 400 μg/ml NmLgtB, 400 μg/ml AtSuSy1, 400 μg/ml GalE, 400 μg/ml Glucose isomerase and 10 mM MgCl₂ in 250 mM phosphate pH 6.0 at 37° C. Conversion from Glucose to lactose is monitored by TLC using a p-anisaldehyde stain. An approximate conversion of 70% is observed after 24 hrs. resulting in the production (predicted) of 3.0 g of lactose product.

### Example 8: Synthesis of Lactose from Glucose

Lactose is produced from Glucose, sucrose, fructose and catalytic amounts of UDP using the four enzymes NmLgtB, AtSuSy1, GalE and Glucose (xylose) isomerase (EC 5.3. 1.5, D-xylose aldose-ketose-isomerase) (FIG. 9G). The reaction mixture is composed of 500 mM Glucose, 500 mM fructose, 500 mM sucrose, 5 mM UDP, 400 μg/ml NmLgtB, 400 μg/ml AtSuSy1, 400 μg/ml GalE, 400 μg/ml Glucose isomerase and 10 mM MgCl₂ in 250 mM phosphate pH 6.0 at 37° C. Conversion from Glucose to lactose is monitored by TLC using a p-anisaldehyde stain. An approximate conversion of 70% is observed after 24 hrs. resulting in the production (predicted) of 3.0 g of lactose product.

### Example 9: Synthesis of LSTa from LNT

LSTa was produced from LNT, 3′-SL, and catalytic amounts of CMP using the three enzymes ST3GAL4, ST3GAL3 and Lactase. The reaction mixture was composed of 5 mM LNT, 25 mM 3′-SL, 5 mM CMP, 300 μg/ml ST3GAL4, 300 μg/ml ST3GAL3, 300 μg/ml lactase and 20 mM MgCl₂ in 25 mM Phosphate pH 6.0 at 37° C. Formation of LSTa was confirmed by comparing it with the commercially purchased LSTa (purchased from Biosynth) via silica gel TLC using a butanol/water/acetic acid mobile phase system (4:2.5:2) with Rf (LSTa) 0.16 and Rf (LNT) 0.33Sss.

Further optimization of LSTa synthesis was performed by varying buffers (50 mM NaOAc; pH 5.5 and 50 mM Na₃PO₄; pH 6.0), equivalents of 3′-SL (500 mM and 1M), CMP (5 mM and 25 mM) at 250 mM LNT with and without lactase in the reaction mixture. It was observed that presence of lactase and using higher concentration of 3′-SL (1M as compared to 500 mM) led to greater conversion LSTa (˜20-25% based on TLC). Both 5 mM and 25 mM CMP reactions gave similar results. Both NaOAc buffer (pH 5.5) and Na₃PO₄ (pH 6.0) buffer gave similar results as well.

### Example 10: Synthesis of LSTb from LNT

LSTb is produced from LNT, 3′-SL, and catalytic amounts of CMP using the five enzymes ST3GAL4, ST3GAL3, ST6GALNAC5, Lactase and α2-3 Neuraminidase S. The reaction mixture is composed of 25 mM LNT, 60 mM 3′-SL, mM CMP, 400 μg/ml ST3GAL4, 400 μg/ml ST3GAL3, 400 μg/ml ST6GALNAC5, 400 μg/ml lactase. 100 μg/ml α2-3 Neuraminidase S and 10 mM MgCl₂ in 20-100 mM buffer in the pH range of 6.5 to 7.5 at 37° C.

### Example 11: Glycosylation Enzymes

The enzymes in Table 1 are used in the methods of the invention. Other enzymes are disclosed herein. Table 1 provides the E number, enzyme name, E.C. number, and an example of an enzyme that may be used. Table 1 may refer to enzymes or genes encoding the enzymes.

TABLE 1
Selected Enzymes

| Enzyme Name | E.C. No. | Examples |
|---|---|---|
| Hexosyl transferase | EC 2.4.1 | β-1,3-galactosyl transferase |
| | | from Chromobacterium |
| | | violaceum |
| | | (Cvb3GalT) |
| β-N-acetylhexosaminidase | EC 3.2.1.52 | Bbh1 |
| Glycosyl transferase | EC 2.4 | sucrose synthase |
| Sucrose synthase | EC 2.4.1.13 | sucrose synthase from |
| | | Arabidopsis thaliana |
| | | (AtSuSy 1) |
| Epimerase | EC 5.1.3.2 | GalE |
| | | UDP-glucose 4-epimerase |
| Hexosyl transferase | EC 2.4.1.22 | B4GALT1 |
| Not an enzyme: cofactor | N/A | α-lactalbumin |
| for B4GALT1 | | |
| Hexosyl transferase | EC 2.4.1 | β-1,4-galactosyltransferase |
| | | from Neisseria meningitidis |
| | | (NmLgtB) |
| Hexosyl transferase | EC 2.4.1 | B3GALT5 |
| Sialyl transferase | EC 2.4.99 | ST3GAL3 |
| Sialyl transferase | EC 2.4.99 | ST3GAL4 |
| Sialyl transferase | EC 2.4.99 | ST6GAL1 |
| Sialyl transferase | EC 2.4.99 | ST6GALNAC5 |
| Glycosidase | 3.2.1 | Lactase |
| Fucosyl transferase | EC 2.4.1 | Te2FT |
| Fucosyl transferase | EC 2.4.1 | HmFucT |
| Fucosyl transferase | EC 2.4.1 | Hp34FT |
| Fucosyl transferase | EC 2.4.1 | FUT9 |
| Fucosyl transferase | EC 2.4.1 | FUT1 |
| Fucosyl transferase | EC 2.4.1 | FUT3 |
| Galactose isomerase (L- | | GsAI (Gene: araA) |
| arabinose isomerase) | | |
| Fucosidase | | See examples disclosed herein |
| Glucosaminyl (N-acetyl) | | GCNT2 |
| transferase 2 | | |
| Oxidase | | See examples disclosed herein |
| Isomerase | | See examples disclosed herein |
| Hydrolase | | See examples disclosed herein |
| Neuraminidase | | See examples disclosed herein |

### Example 12: In Situ GDP-L-fucose Production of 2′-FL Synthesis with Pyruvate-AcK/PyrOx+FDH Regeneration

Enzymes E1, E1′, E2, E3, E4, E5, E6, E7, E8, E9, E10, E11, and E12 used in Example 1 are immobilized. All reagents and cofactors, buffer, salts are added at the beginning. The enzymes are immobilized, and in an example of a flow reactor (e.g. packed bed reactor) the above mixture of reagents is flowed through this flow reactor. Glucose, fructose, or a mixture of glucose and fructose along with feed stock and immobilized enzymes are introduced into the reactor. The FIG. 15 depicted conversions are carried out to provide 2′FL or another fucosylated oligosaccharide or fucosylated antibody-glycan conjugate.

D-mannose isomerase converts glucose or converts fructose to mannose. Glk (HK) coverts mannose into mannose-6-phosphate, along with ATP conversion to ADP. RfbK (ManB) converts mannose-6-phosphate to mannose-1-phosphate. RfbM (ManC) converts mannose-1-phosphate to GDP-D-Man, along with conversion of GTP to PPi. Gmd converts GDP-D-Man to GDP-4-keto-6-deoxymannose. GFS (WcaG) converts GDP-4-keto-6-deoxymannose to GDP-L-fucose along with conversion of NADPH to NADP+. Fucosyl transferase converts lactose and GDP-L-fucose to 2′FL. In the same reactor, the following reactions occur. PmPpa converts inorganic diphosphate to inorganic phosphate. AcK and pyruvate oxidase converts pyruvate and inorganic phosphate (generated by PmPpa) to CO₂ and peroxide (with catalase converting the CO₂ and peroxide to oxygen and water). FDH converts formate to CO₂ along with conversion of NADP+ to NADPH. Also in the same reactor, GDP is converted to GTP. 2′FL is obtained. See FIG. 15.

### Example 13: In Situ GDP-L-fucose Production for 2′-FL Synthesis with Pyruvate-AcK/PyrOx+PtxD Regeneration

Enzymes (in immobilized form) E1, E1′, E2, E3, E4, E5, E6, E7, E8′, E9, E10, E11, and E12 are combined with feedstock to carry out the reactions depicted in FIG. 16 to provide 2′FL or another fucosylated oligosaccharide or fucosylated antibody-glycan conjugate.

### Example 14: In Situ GDP-L-Fucose Production for 2′-FL Synthesis with PolyP-PPK+FDH Regeneration

Enzymes (in immobilized form) E1, E1′, E2, E3, E4, E5, E6, E7, E8, E9′, and E12 are combined with feedstock to carry out the reactions depicted in FIG. 17 to provide 2′FL or another fucosylated oligosaccharide or fucosylated antibody-glycan conjugate.

### Example 15: In Situ GDP-L-Fucose Production for 2′-FL Synthesis with PolyP-PPK+PtxD Regeneration

Enzymes (in immobilized form) E1, E1′, E2, E3, E4, E5, E6, E7, E8′, E9′, and E12 are combined with feedstock to carry out the reactions depicted in FIG. 18 to provide 2′FL or another fucosylated oligosaccharide or fucosylated antibody-glycan conjugate.

### Example 16: Enzymes

The enzymes in Table 2 are used in the methods of the invention. Table 2 provides the E number used in FIGS. 15-18, enzyme name, E.C. number, and an example of an enzyme that may be used. Table 2 may refer to enzymes or genes encoding the enzymes.

TABLE 2
Integrated Fucosylation Pathway Enzymes

| E# | Enzyme Name | E.C. No. | Examples |
|---|---|---|---|
| E10 | Acetate Kinase | 2.7.2.1 | AcK |
| E11 | Catalase | 1.11.1.6 | Cat |
| E1 | D-mannose isomerase | 5.3.1. | D-Mannose-2-epimerase, |
| | | 5.3.1.7 | |
| E1’ | Epimerase-isomerase | EC 5.3.1.7 | AGEase (P. geniculata) |
| | | EC 5.3.1.15, | |
| | | EC 5.3.1.11 | |
| E8 | Formate dehydrogenase | 1.17.1.9 | FDH |
| E7 | Fucosyl transferase | 2.4.1 | HmFucT, Hp34FT, FUT9 |
| E6 | GDP-Fucose Synthase | 1.1.1.271 | GFS (WcaG) |
| E4 | GDP-Man Pyrophosphorylase | 2.7.7.13 | RfbM (ManC) |
| E5 | GDP-Mannose-4,6- | 4.2.1.47 | Gmd |
| | dehydratase | | |
| E2 | Hexokinase | 2.7.1.1 | Glucokinase (GlK) |
| E12 | Inorganic Pyrophosphatase | 3.6.1.1 | PmPpa |
| E8' | Phosphite dehydrogenase | 1.20.1.1 | PtxD (PTDH) |
| E3 | Phosphomannomutase | 5.4.2.8 | RfbK (ManB) |
| E9' | Polyphosphate Kinase | 2.7.4.1 | PPK2 |
| E9 | Pyruvate Oxidase | 1.2.3.3 | PyrOx |

https://www.researchgate.net/figure/Production-of-D-mannose-from-D-fructose-and-D-glucose-using-different-enzymes-MIase_fig1_336675669. Lui et al., Foods, 9(12), 1809.

All publications and patent documents disclosed or referred to herein are incorporated by reference in their entirety.

The foregoing description has been presented only for purposes of illustration and description. This description is not intended to limit the invention to the precise form disclosed. It is intended that the scope of the invention be defined by the claims appended hereto.

1. A method for producing a glycosylated principal product, comprising the steps of:
a. contacting a catalytic amount of a sugar-nucleotide donor and a stoichiometric amount of an acceptor in the presence of a transferase to obtain a glycosylated principal product and a catalytic amount of a nucleotide; and
b. regenerating said nucleotide into a regenerated sugar-nucleotide donor by contacting said catalytic amount of said nucleotide with a stoichiometric amount of a sugar donor precursor in the presence of a transferase to obtain said regenerated sugar-nucleotide donor and a secondary product.

2. The method of claim 1, further comprising
a. contacting a catalytic amount of said regenerated sugar-nucleotide donor and a said stoichiometric amount of an acceptor in the presence of a transferase to obtain said glycosylated principal product and said catalytic amount of a nucleotide; and
b. regenerating said nucleotide into said regenerated sugar-nucleotide donor by contacting said catalytic amount of said nucleotide with said stoichiometric amount of said sugar donor precursor in the presence of a transferase to obtain said regenerated sugar-nucleotide donor and said secondary product.

3. A method for producing a glycosylated principal product comprising the step of adding a sugar donor precursor, an acceptor, a transferase, and a catalytic amount of a nucleotide to obtain a glycosylated principal product and a secondary product.

4. The method according to claim 3, wherein said sugar donor precursor and said nucleotide provides a sugar-nucleotide donor.

5. The method according to claim 1, wherein said sugar donor precursor and said nucleotide provides a sugar-nucleotide donor precursor and wherein an auxiliary enzyme and said sugar-nucleotide donor precursor provides said sugar-nucleotide donor.

6.-27. (canceled)

28. A method for producing a galactosylated principal product, comprising the steps of:
a. contacting a catalytic amount of a sugar-nucleotide donor and a stoichiometric amount of an acceptor to obtain a galactosylated principal product and a catalytic amount of a nucleotide; and
b. regenerating said nucleotide into a regenerated sugar-nucleotide donor by contacting said catalytic amount of said nucleotide and a stoichiometric amount of a sugar donor precursor to obtain said regenerated sugar-nucleotide donor and a secondary product.

29. The method of claim 28, further comprising
a. contacting a catalytic amount of said regenerated sugar-nucleotide donor and said stoichiometric amount of an acceptor to obtain said glycosylated principal product and said catalytic amount of a nucleotide; and
b. regenerating said nucleotide into said regenerated sugar-nucleotide donor by contacting said catalytic amount of a nucleotide and said stoichiometric amount of a sugar donor precursor to obtain said regenerated sugar-nucleotide donor and said secondary product.

30. A method for producing a galactosylated principal product comprising the step of adding a sugar donor precursor, an acceptor, a transferase, and a catalytic amount of a nucleotide to obtain a glycosylated principal product and a secondary product.

31. The method according to claim 28, wherein said acceptor is lacto-N-triose II (LNTII), glucose, or galactooligosaccharide (GOS).

32.-45. (canceled)

46. A method for producing a sialylated principal product, comprising the steps of:
a. contacting a catalytic amount of a sugar-nucleotide donor and a stoichiometric amount of an acceptor to obtain a sialylated principal product and a catalytic amount of a nucleotide; and
b. regenerating said nucleotide into a regenerated sugar-nucleotide donor by contacting a catalytic amount of said nucleotide and a stoichiometric amount of a sugar donor precursor to obtain said regenerated sugar-nucleotide donor and a secondary product.

47. The method of claim 46, further comprising:
a. contacting a catalytic amount of said regenerated sugar-nucleotide donor and said stoichiometric amount of an acceptor to obtain said glycosylated principal product and said catalytic amount of a nucleotide; and
b. regenerating said nucleotide into said regenerated sugar-nucleotide donor by contacting said catalytic amount of a nucleotide and said stoichiometric amount of a sugar donor precursor to obtain said regenerated sugar-nucleotide donor and said secondary product.

48. A method for producing a sialylated principal product comprising the step of adding a sugar donor precursor, an acceptor, a transferase, and a catalytic amount of a nucleotide to obtain a sialylated principal product and a secondary product.

49. The method according to claim 46, wherein said sugar donor precursor is 3′-sialyllactose.

50-69. (canceled)

70. A method for producing a fucosylated principal product, comprising the steps of:
a. contacting a catalytic amount of a sugar-nucleotide donor to a stoichiometric amount of an acceptor to obtain a fucosylated principal product and a catalytic amount of a nucleotide; and
b. regenerating said nucleotide into a regenerated sugar-nucleotide donor by contacting a catalytic amount of said nucleotide with a stoichiometric amount of a sugar donor precursor to obtain said regenerated sugar-nucleotide donor and a secondary product.

71. The method of claim 70, further comprising
a. contacting a catalytic amount of said regenerated sugar-nucleotide donor and said stoichiometric amount of an acceptor to obtain said glycosylated principal product and said catalytic amount of a nucleotide; and
b. regenerating said nucleotide into said regenerated sugar-nucleotide donor by contacting said catalytic amount of a nucleotide and said stoichiometric amount of a sugar donor precursor to obtain said regenerated sugar-nucleotide donor and said secondary product.

72. A method for producing a fucosylated principal product comprising the step of adding a sugar donor precursor, an acceptor, a transferase, and a catalytic amount of a nucleotide to obtain a fucosylated principal product and a secondary product.

73. The method according to claim 70, wherein said sugar donor precursor is a 2′-fucosyllactose.

74.-95. (canceled)

96. A method for producing a N-acetylglucosaminylated principal product, comprising the steps of:
a. contacting a catalytic amount of a sugar-nucleotide donor and a stoichiometric amount of an acceptor to obtain a N-acetylglucosaminylated principal product and a catalytic amount of a nucleotide; and
b. regenerating said nucleotide into a regenerated sugar-nucleotide donor by contacting a catalytic amount of said nucleotide and a stoichiometric amount of a sugar donor precursor to obtain said regenerated sugar-nucleotide donor and a secondary product.

97. The method of claim 1, further comprising:
a. contacting a catalytic amount of said regenerated sugar-nucleotide donor and said stoichiometric amount of an acceptor to obtain said glycosylated principal product and said catalytic amount of a nucleotide; and
b. regenerating said nucleotide into said regenerated sugar-nucleotide donor by contacting said catalytic amount and a nucleotide with said stoichiometric amount of a sugar donor precursor to obtain said regenerated sugar-nucleotide donor and said secondary product.

98. A method for producing a N-acetylglucosaminylated principal product comprising the step of adding a sugar donor precursor, an acceptor, a transferase, and a catalytic amount of a nucleotide to obtain a N-acetylglucosaminylated principal product and a secondary product.

99. The method according to claim 96, wherein said sugar donor precursor is lacto-N-biose.

100.-129. (canceled)

130. A machine configured for said method of claim 1.

131. The machine according to claim 130, wherein said method occurs within a single reaction vessel.

132. A process for producing a fucosylated oligosaccharide or a fucosylated antibody-glycan conjugate, comprising the steps of.
a. contacting glucose or fructose in the presence of an enzyme that converts said fructose or glucose to mannose;
b. contacting said mannose with an enzyme that converts mannose to mannose-6-phosphate;
c. contacting said mannose-6-phosphate with an enzyme that converts mannose-6-phosphate to mannose-1-phosphate;
d. contacting said mannose-1-phophate with an enzyme that converts said mannose-1-phophate to GDP-D-mannose;
e. contacting said GDP-D-mannose with an enzyme that converts GDP-D-mannose to GDP-4-keto-6-deoxymannose;
f. contacting said GDP-4-keto-6-deoxymannose with an enzyme that converts said GDP-4-keto-6-deoxymannose to GDP-L-fucose;
g. contacting said GDP-L-fucose with a disaccharide, an oligosaccharide or an antibody-glycan conjugate with an enzyme that fucosylates said disaccharide, oligosaccharide or said antibody-glycan conjugate; and
h. obtaining a fucosylated disaccharide, oligosaccharide or a fucosylated antibody-glycan conjugate.
wherein each of said enzymes is immobilized and wherein said process contains a set of regeneration enzyme systems to convert ADP to ATP, PPi to Pi, GDP to GTP, and NADP+ to NADPH.

133.-153. (canceled)
